(19) United States
(12) Patent Application Publication
(10) Pub. No.: US 2025/0205162 A1
(43) Pub. Date: Jun. 26, 2025

(54) SYSTEMS AND METHODS FOR SMALL MOLECULE ENCAPSULATION

(71) Applicant: Tecton Group, LLC, Alexandria, LA (US)

(72) Inventors: Kattesh V. Katti, Columbia, MO (US); Alice Raphael Karikachery, Columbia, MO (US); Kavita K. Katti, Columbia, MO (US); Velaphi C. Thipe, Columbia, MO (US); Michael A. Schmidt, Boulder, CO (US); Alton Michael Chesne, Alexandria, LA (US); Udaiyan Jatar, Atlanta, GA (US)

(21) Appl. No.: 18/942,098

(22) Filed: Nov. 8, 2024

Related U.S. Application Data
(63) Continuation of application No. PCT/US2023/021578, filed on May 9, 2023.
(60) Provisional application No. 63/340,820, filed on May 11, 2022.
(60) Provisional application No. 63/453,958, filed on Mar. 22, 2023.

(51) Int. Cl. A61K 9/50; A61K 31/22; A61K 31/522; A61K 31/706

(52) U.S. Cl. CPC A61K 9/5052; A61K 9/5089; A61K 31/22; A61K 31/522; A61K 31/706

(57) ABSTRACT

Disclosed herein are systems and methods for the encapsulation of small molecules (e.g., ketone molecules) with plant-derived proteins, such as for dietary supplements.

### CROSS REFERENCE

This application is a continuation of International Application No. PCT/US2023/021578, filed May 9, 2023, which claims the benefit of U.S. Provisional Application No. 63/340,820, filed May 11, 2022, and U.S. Provisional Application No. 63/453,958, filed Mar. 22, 2023, which applications are incorporated herein by reference in their entireties.

### BACKGROUND OF THE INVENTION

The health and environmental benefits of plant-derived protein food products are broadly recognized. To meet the rising demand for vegetarian and vegan dietary products, scientists have engaged in efforts to derive plant-derived protein products to replace animal-derived protein products, for example in the incorporation of dietary supplements into food products for enhanced bio-availability. There exists an unmet need for a cost-effective method to deliver dietary supplements such as ketones, nicotinamide ribosides (NR), caffeine or theacrine without the use of animal-derived protein products.

### SUMMARY OF THE INVENTION

Disclosed herein is a method of encapsulating a plurality of small molecules, the method comprising: obtaining plant-derived protein and one or more phytochemicals; mixing the plant-derived protein and the one or more phytochemicals with the plurality of small molecules in water to produce a mixture comprising a plurality of particles comprising a plurality of encapsulated small molecules. The plurality of small molecules can comprise a plurality of ketone molecules. The plurality of small molecules can comprise a plurality of nicotinamide riboside molecules or nicotinamide riboside analogs. The plurality of small molecules can comprise a plurality of caffeine molecules. The plurality of small molecules can comprise a plurality of theacrine molecules. The plant-derived protein can be from a first source and the one or more phytochemicals can be from a second source that differs from the first source. The first source can be from a first plant species and the second source can be from a second plant species. The plant-derived protein can be pea protein. The plant-derived protein can be water soluble. The plant-derived protein can be banana protein, okra protein, or bean protein. The bean protein can be soybean protein. The phytochemicals can be derived from mango, tea leaf, okra, berry, or grape. The phytochemicals can be derived from mango peel powder, dried tea leaf, okra extract powder, berry extract, or grape extract. The one or more phytochemicals can comprise a mangiferin, a catechin, or a quercetin. The one or more phytochemicals can comprise quercetin and the weight ratio of the one or more phytochemicals to the plant-derived protein can be between 1:10000 and 1:40000. The one or more phytochemicals can comprise quercetin and the weight ratio of the one or more phytochemicals to the plant-derived protein can be between 1:22000 and 1:23000. The one or more phytochemicals can comprise mangiferin, and the weight ratio of the one or more phytochemicals to the plant-derived protein can be between 1:20 and 1:60. The one or more phytochemicals can comprise mangiferin, and the weight ratio of the one or more phytochemicals to the plant-derived protein can be between 1:30 and 1:50. The one or more phytochemicals can comprise catechin, wherein the catechin is epigallocatechin gallate. The weight ratio of the epigallocatechin gallate to the plant-derived protein can be between 1:200 and 1:400. The one or more phytochemicals can comprise a polyphenol. The polyphenol can comprise one or more of a flavonoid and resveratrol. The weight ratio of the one or more phytochemicals to the plant-derived protein can be between 1:300 and 1:500. The mixture can comprise a dry-weight ratio of the plurality of ketone molecules to plant-derived protein between 1:1 and 1:5. The mixture can comprise a weight ratio of plant-derived protein to water between 1:50 and 1:100 or between 1:10 and 1:100.

The method can further comprise mixing the mixture with ethanol. Mixing the mixture with ethanol to produce particles comprising ketone molecules or NR molecules can be performed by adding the ethanol to the soluble fraction at a 1:10 to 1:1 ratio by volume of the ethanol to the soluble fraction. Mixing the mixture with ethanol to produce particles comprising ketone molecules or NR molecules can be performed by adding the ethanol to the soluble fraction at a 1:10 to 1:5 ratio by volume of the ethanol to the soluble fraction. Mixing the mixture with ethanol to produce particles comprising ketone or NR molecules can be performed by adding the ethanol to the soluble fraction at a 1:4 to 1:2 ratio by volume of the ethanol to the soluble fraction. The method can comprise removing the ethanol. Removing the ethanol can comprise evaporation of the ethanol at from 25° C. to 40° C.

The particles can have an average diameter greater than 80 nm. The particles can have an average diameter less than 700 nm. The particles can have an average diameter between 100 nm and 500 nm. The particles can have an average diameter between 300 nm and 500 nm. Removing the ethanol can comprise evaporation of the ethanol at from 25° C. to 40° C.

The method can further comprise cross-linking the phytochemicals to the plant protein. The phytochemical can be cross-linked to the plant protein via an imine linkage. The cross-linking can be performed without an addition of an aldehyde. The aldehyde can be glutaraldehyde. The cross-linking can occur at one or more functional amino acid groups of the plant protein. The functional amino acid group can comprise a primary amino group. The functional amino acid groups can comprise lysine.

The mixture can comprise a soluble fraction and an insoluble fraction. The method can comprise, separating the soluble fraction from the insoluble fraction. The soluble fraction can be mixed with ethanol. Separating the soluble fraction from the insoluble fraction can comprise filtering the insoluble fraction from the soluble fraction. The plurality of small molecules can comprise 1,3 Dihydroxypropan-2-yl (R)-3-hydroxybutanoate. At least 50% of the small molecules can be 1,3 Dihydroxypropan-2-yl (R)-3-hydroxybutanoate. At least 50% of the small molecules can comprise a glyceryl backbone conjugated to a 3-hydroxybutanoate.

In some embodiments, the plurality of small molecules can comprise nicotinamide riboside. In some embodiments, at least 50% of the small molecules are nicotinamide riboside. In some embodiments, the plurality of small molecules can comprise caffeine. In some embodiments, at least 50% of the small molecules are caffeine. In some embodiments, the plurality of small molecules can comprise theacrine. In some embodiments, at least 50% of the small molecules are theacrine.

Mixing can be performed from 20° C. to 30° C. Mixing can be performed at a pH of 3.5 to 11.0. Mixing can be performed at a pH of 3.5 to 7.0, 5.0 to 9.0, or 7.0 to 11.0.

The plant-derived protein can be water soluble. The plant-derived protein can be at least 95% water soluble. The plant-derived protein can be at least 98% water soluble. The plant-derived protein can be at least 99% water soluble.

Disclosed herein is a nano-encapsulated composition formed by the above method. Disclosed herein is a composition comprising a small molecule encapsulated within a plant protein. The plant protein can be pea protein. The plant protein can be okra protein, banana protein, or bean protein. The plant protein can be cross-linked with one or more phytochemicals. The one or more phytochemicals can be derived from mango, tea leaf, okra, berry, or grape. The one or more phytochemicals can be derived from mango peel powder, dried leaf, okra extract powder, berry extract, or grape extract. The one or more phytochemicals can comprise a mangiferin, a catechin, or a quercetin. The catechin can be epigallocatechin gallate. The one or more phytochemicals can comprise a polyphenol. The polyphenol can comprise one or more of a flavonoid and resveratrol. The one or more phytochemicals can be cross-linked to the plant protein via an imine linkage. The cross-linking can occur at functional amino acid groups of the plant protein. The functional amino acid groups can comprise a primary amine. The small molecule can comprise a ketone molecule. The ketone molecule can comprise 1,3 Dihydroxypropan-2-yl (R)-3-hydroxybutanoate. The small molecule can comprise a nicotinamide riboside molecule or an analog thereof. The small molecule can comprise a caffeine molecule. The small molecule can comprise a theacrine molecule. The particle can have an average diameter greater than 80 nm. The particle can have an average diameter less than 700 nm. The particle can have an average diameter between 100 nm and 500 nm. The particle can have a diameter of between 300 nm and 500 nm. The composition can have a phenol content greater than 10 mg GAE/g. The composition can have a phenol content less than or equal to 350 mg GAE/g. The composition can have a phenol content between 10 and 500 mg GAE/g. The composition can have a phenol content between 100 and 400 mg GAE/g. The composition can have a phenol content between 200 and 300 mg GAE/g. In some embodiments, the composition does not comprise glutaraldehyde.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a UV-visible spectra of ketone molecules at 2 mg/mL.

FIG. 2 shows a UV-visible absorption spectra of NR at 0.2 mg/mL.

FIG. 3 shows ¹H NMR of ketone molecules in D₂O.

FIG. 4 shows ¹H NMR of NR molecules in D₂O.

FIG. 5 shows ¹³C NMR of ketone molecules in D₂O.

FIG. 6 shows ¹³C NMR of ketone molecules in D₂O.

FIG. 7 shows ¹³C NMR of ketone molecules in D₂O.

FIG. 8 shows ¹³C NMR of NR molecules in D₂O.

FIG. 9 shows electrospray ionization-mass spectrometry (ESI-MS) spectra of ketone molecules.

FIG. 10 shows electrospray ionization-mass spectrometry (ESI-MS) spectra of NR molecules.

FIG. 11 shows a graph of GC-MS analysis of ketone molecules.

FIG. 12 shows graphs of LC-MS analysis of ketone molecules.

FIG. 13 shows ketone molecule CD spectrum (wavelength versus ellipticity) wherein the positive peak indicates a R/D configuration of the ketone molecules.

FIG. 14A shows NR molecule CD spectrum (wavelength versus ellipticity) wherein the negative peak indicates a S/L configuration of the NR molecules.

FIG. 14B shows graphs of LC-MS analysis of caffeine (CA) molecule.

FIG. 14C shows graphs of LC-MS analysis of theacrine (TH) molecule.

FIG. 15 shows a schematic of the preparation of KM-protein nanoparticles by a desolvation method.

FIG. 16 shows a schematic of the preparation of NR-protein nanoparticles by a desolvation method.

FIG. 17A shows the UV-visible absorption spectra of water-soluble pea protein at 0.2 mg/mL.

FIG. 17B shows the fluorescence emission spectra of water-soluble pea protein at 0.2 mg/mL at 285 nm excitation.

FIG. 18 shows MALDI-MS analysis of water-soluble pea protein.

FIG. 19 shows TEM images of ketone molecules encapsulated by pea protein nanoparticles, produced by ethanol desolvation, crosslinked with mango peel derived phytochemicals.

FIG. 20A shows TEM images of NR molecules encapsulated by pea protein nanoparticles, produced by ethanol desolvation, crosslinked with mango peel derived phytochemicals.

FIG. 20B shows TEM image of CA molecules encapsulated by pea protein nanoparticles, crosslinked with mango peel derived phytochemicals.

FIG. 20C shows TEM image of TH molecules encapsulated by pea protein nanoparticles, crosslinked with mango peel derived phytochemicals.

FIG. 21 shows fluorescence emission spectra of KM-PP-MP-NP and PP-MP-NP at 285 nm excitation.

FIG. 22A shows cell viability of human aortic endothelial cells (HAEC) after 24, 48 and 72 hours post incubation with KM. The control is untreated cells.

FIG. 22B shows cell viability of human aortic endothelial cells (HAEC) after 24, 48 and 72 hours post incubation with KM encapsulated pea protein nanoparticles with mango peel crosslinking (KM-PP-MP-NP). KM concentration in KM-PP-MP-NP used for plotting. The control is untreated cells.

FIG. 23 shows TEM images of KM encapsulated Pea Protein Nanoparticles by ethanol desolvation method and Tea Extract crosslinking (KM-PP-TE-NP).

FIG. 24A shows TEM images of NR encapsulated Pea Protein Nanoparticles by ethanol desolvation method and Tea Extract crosslinking (NR-PP-TE-NP).

FIG. 24B shows TEM image of CA encapsulated Pea Protein Nanoparticles by Tea Extract crosslinking (CA-PP-TE-NP).

FIG. 24C shows TEM image of TH encapsulated Pea Protein Nanoparticles by Tea Extract crosslinking (TH-PP-TE-NP).

FIG. 25 shows fluorescence emission spectra of KM-PP-TE-NP and PP-TE-NP at 285 nm excitation.

FIG. 26A shows cell viability of human aortic endothelial cells (HAEC) after 24, 48 and 72 hours post incubation with KM. The control is untreated cells.

FIG. 26B shows cell viability of human aortic endothelial cells (HAEC) after 24, 48 and 72 hours post incubation with KM encapsulated pea protein nanoparticles with tea extract crosslinking (KM-PP-TE-NP). KM concentration in KM-PP-MP-NP used for plotting. The control is untreated cells.

FIG. 27A shows TEM images of KM encapsulated Pea Protein Nanoparticles by Berry extract crosslinking (KM-PP-BE-NP).

FIG. 27B shows TEM images of KM encapsulated Pea Protein Nanoparticles by Grape extract crosslinking (KM-PP-GE-NP).

FIG. 27C shows TEM image of CA encapsulated Pea Protein Nanoparticles by Berry Extract crosslinking (CA-PP-BE-NP).

FIG. 27D shows TEM image of TH encapsulated Pea Protein Nanoparticles by Berry Extract crosslinking (TH-PP-BE-NP).

FIG. 28 shows total polyphenol content of plant-based crosslinked KM encapsulated pea protein nanoparticles. Gallic acid standard curve (a) and total polyphenol content determined by Folin-Ciocalteu method (b) Bars represent the mean+SEM of three independent experiments carried out in duplicate. GAE=gallic acid equivalent

FIG. 29 shows Okra mucilage MALDI-MS analysis.

FIG. 30 shows UV-visible absorption spectra of okra mucilage.

FIG. 31 shows TEM images of KM encapsulated Okra Extract Nanoparticles (KM-OE-NP).

FIG. 32 shows TEM images of NR encapsulated Okra Extract Nanoparticles (NR-OE-NP).

FIG. 33 shows UV-visible absorption spectra of water-soluble Soy Protein Isolate (SPI) (0.2 mg/ml).

FIG. 34 shows Soy Protein Isolate (SPI) MALDI-MS analysis.

FIG. 35 shows TEM images of KM encapsulated Soy Protein Isolate Nanoparticles by ethanol desolvation method (KM-SPI-NP).

FIG. 36 shows pea protein KM nanoparticle synthesis utilizing biocompatible plant-based crosslinking agents.

FIG. 37 shows TEM images of KM-encapsulated pea protein nanoparticles by ethanol desolvation method and plant-based crosslinking (a—mangiferin from mango peel extract, b—EGCG from tea extract, c—quercetin from black berry extract and d—resveratrol from grape extract).

FIG. 38 shows the creation of plant protein-phytochemical platform through interactions of phytochemicals with protein amino side chains.

FIG. 39 shows ketone molecule bonding with plant-derived proteins and phytochemicals.

FIG. 40 shows ¹H NMR of KM in D₂O.

FIG. 41 shows ¹H NMR of water-soluble Pea Protein (PP) in D₂O.

FIG. 42 shows ¹H NMR of mangiferin in CD₃OD.

FIG. 43 shows ¹H NMR (expanded) of mangiferin and water-soluble Pea Protein (PP) reaction mixture in CD₃OD/D₂O (arrows indicate change in proton).

FIG. 44 shows ¹H NMR (expanded) of mangiferin, water soluble Pea Protein (PP) and KM reaction mixture in CD₃OD/D₂O.

FIG. 45 shows ¹H NMR of epigallocatechin gallate (EGCG) in D₂O.

FIG. 46 shows ¹H NMR (expanded) of epigallocatechin gallate (EGCG) and water-soluble Pea Protein (PP) reaction mixture in D₂O (arrows indicate change in proton).

FIG. 47 shows ¹H NMR (expanded) of epigallocatechin gallate (EGCG), water soluble Pea Protein (PP) and KM reaction mixture in D₂O.

FIG. 48 shows ¹H NMR of quercetin in CD₃OD.

FIG. 49 shows H NMR (expanded) of quercetin and water-soluble Pea Protein (PP) reaction mixture in CD₃OD/D₂O (arrows indicate change in proton).

FIG. 50 shows ¹H NMR (expanded) of quercetin, water-soluble Pea Protein (PP) and KM reaction mixture in CD₃OD/D₂O.

FIG. 51 shows ¹H NMR of resveratrol in CD₃OD.

FIG. 52 shows ¹H NMR (expanded) of resveratrol and water-soluble Pea Protein (PP) reaction mixture in CD₃OD/D₂O (arrows indicate change in proton).

FIG. 53 shows ¹H NMR (expanded) of resveratrol, water-soluble Pea Protein (PP) and KM reaction mixture in CD₃OD/D₂O.

FIG. 54 shows the amino acid composition of pea protein (gram per 100 g protein).

FIG. 55 shows the amino acid composition of okra extract (gram per 100 g protein).

FIG. 56 shows the amino acid composition of soy protein isolate (gram per 100 g protein).

FIG. 57 shows the comparison of the amino acid composition of pea protein, okra extract, and soy protein isolate (gram per 100 g protein).

FIG. 58 shows the polyphenolic composition distribution (milligram per gram dry matter) of mango peel powder obtained from mango fruit.

FIG. 59 shows the polyphenolic composition distribution (milligram per gram dry matter) of black tea leaves.

FIG. 60 shows the anthocyanins and non-anthocyanin phenolic compound composition (microgram per 100-gram dry matter) of Blackberry Extract (BE).

FIG. 61 shows the polyphenolic composition distribution (milligram per gram dry matter) of grape extract.

### DETAILED DESCRIPTION OF THE INVENTION

Plant based proteins are biocompatible, economically viable, and generally safe for use as nutraceuticals or as smart foods/pharmaceuticals. Disclosed here are systems and methods to encapsulate ketone molecules (KM), nicotinamide riboside (NR) molecules, and their analogs to enhance stability, enhance biocompatibility, and transform these types of food supplement(s)/nutraceutical(s) to be biocompatible, target and cell specific in vivo. Disclosed herein are methods and systems to enhance the ability of KM molecules to penetrate biological domain barriers including the blood brain barrier for rapid and sustained release of KM molecules to achieve instant and sustainable energy to the human body. Also disclosed herein are methods and systems for delivery of NR molecules and analogs thereof. Disclosed herein are also analogous systems suitable for encapsulation of other small molecules.

The term “small molecule” as used herein refers to molecules of less than or equal to 900 daltons.

The term “ketone molecule” as described herein refers to a molecule with the structure of Compound I below, wherein R¹, R² and R³ can be H or —C₄H₆O₂ and at least one of R¹, R² and R³ is —C₄H₆O₂

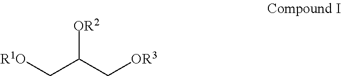

Compound I can comprise any of Compounds II-V below.

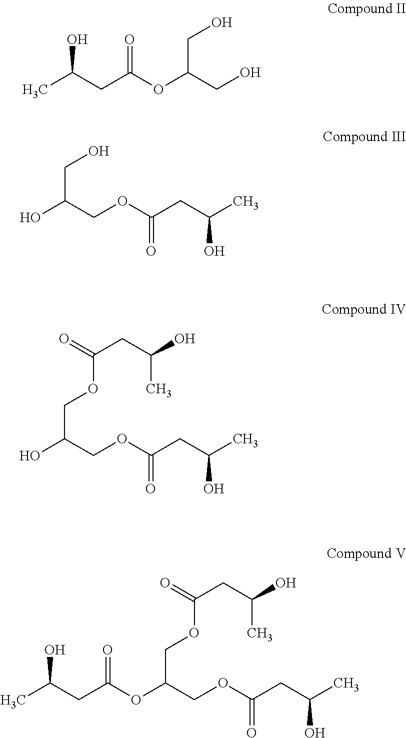

In some embodiments, —C₄H₆O₂ is

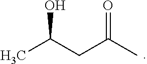

The term “nicotinamide riboside” as described herein refers to a molecule with the structure of Compound VI below.

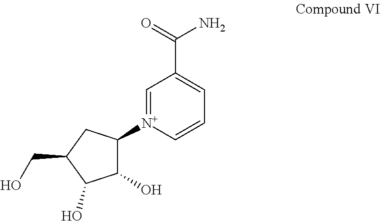 [SMILES: NC(=O)c1ccc[n+]([C@@H]2C[C@H](CO)[C@@H](O)[C@H]2O)c1]

The analogs of nicotiamide riboside molecule can comprise any of the Compounds VII-IX below.

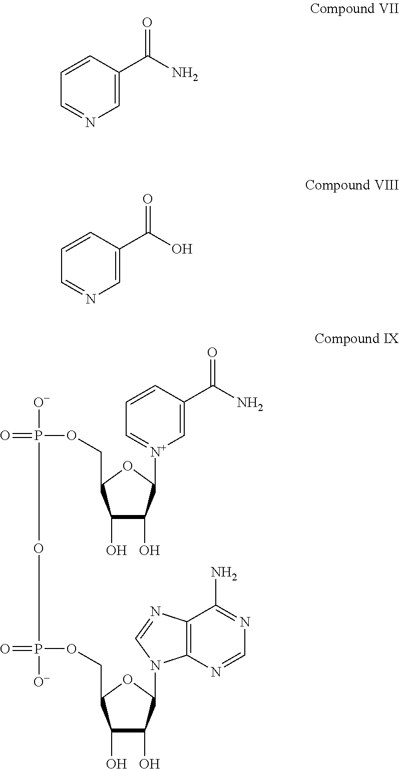

The term “caffeine” as described herein refers to a molecule with the structure of Compound X below.

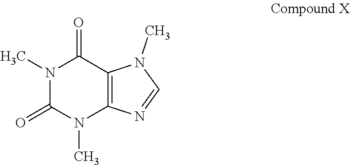

The term “theacrine” as described herein refers to a molecule with the structure of Compound XI below.

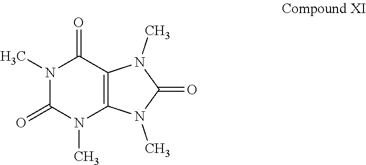 [SMILES: Cn1c(=O)c2c(n(C)c1=O)n(C)c(=O)n2C]

The term “plurality of ketone molecules” as described herein can refer to any plurality of ketone molecules. In some instances, the plurality of ketone molecules are at least 70% Compound II. The plurality of ketone molecules can be greater than or equal to 99%, 98%, 95%, 90%, 85%, 80%, 75%, 70%, 65%, 60% 55% of 50% Compound II. The plurality of ketone molecules can be less than or equal to 50%, 40%, 30%, 20%, 10%, 5%, or 1% Compound III, Compound IV, or Compound V.

The molar ratio of Compound II to a mixture of Compounds III-V in solution can be between 80:20 and 99:1. The molar ratio of Compound II to a mixture of Compounds III-V in solution can be greater than or equal to about 80:20, 85:15, 90:10, 95:5, or 99:1. The molar ratio of Compound II to a mixture of Compounds III-V in solution can be less than or equal to 99:1, 95:5, 90:10, 85:15, or 80:20.

The term “plurality of nicotinamide riboside molecules” as described herein can refer to any plurality of nicotinamide riboside molecules. In some instances, the plurality of NR molecules can be nicotinamide riboside hydrogen maleate. In some instances, the plurality of NR molecules can be nicotinamide riboside chloride. In some instances, the plurality of NR molecules are at least 70% Compound VI. The plurality of NR molecules can be greater than or equal to 99%, 98%, 95%, 90%, 85%, 80%, 75%, 70%, 65%, 60% 55% of 50% Compound VI. The plurality of NR molecules can be less than or equal to 50%, 40%, 30%, 20%, 10%, 5%, or 1% Compound VII, Compound VIII, or Compound IX.

The term “encapsulating” as described herein refers to incorporating a small molecule, such as a ketone, NR, caffeine, or theacrine molecule, into a cross-linked particle, even if the small molecule is not fully surrounded by plant-derived protein or other non-ketone molecules.

An embodiment of this invention encapsulates ketone, NR, caffeine, or theacrine molecules within plant-based proteins from peas, okra, or banana. Ketone, NR, caffeine, or theacrine molecules can be encapsulated within plant-based proteins from peas, okra, or banana using plant-based cross-linking agents including various polyphenols, flavonoids, xanthonoids and functionalized glucose moieties. Methods and systems disclosed herein can utilize the ingredients of plant-based proteins and phytochemicals from peas, okra, banana, mango, tea leaves, berries, and grapes to produce particles comprising plant-based proteins loaded with ketone, NR, caffeine or theacrine molecules. The methods and systems disclosed herein can be used to produce particles that provide rapid and sustainable energy along with a rich group of key nutrients, including potassium, dietary fiber, polyunsaturated fats, essential amino acids, vitamin B6, vitamin B12, magnesium, potassium, fiber, and protein.

Fibers from the peas, banana and okra can contribute to gastrointestinal function and health. The amylose contents in such plant-based protein extracts can help in lowering glycemic index. In addition, the hydrolysis products of pea protein encapsulated ketone, NR, caffeine, or theacrine molecules can generate peptides with bioactivity, for example peptides capable of angiotensin I-converting enzyme inhibitor activity and antioxidant activity. The plant-derived protein particles described herein can provide polyphenolics with antioxidant, anticarcinogenic, or hypocholesterolemia activities. The plant-derived protein encapsulated ketone particles can provide prebiotic effects, such as from the galactose oligosaccharides, in the large intestine.

### Ketone Molecules (KM)

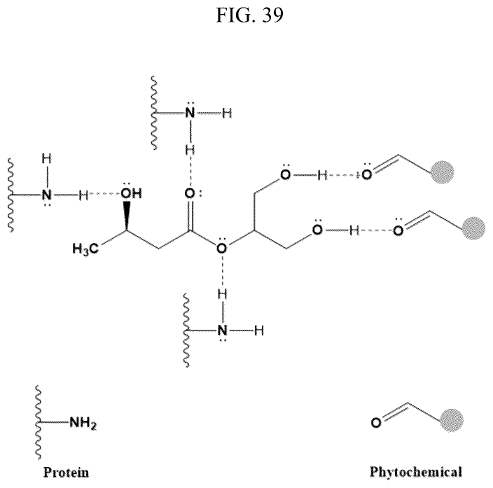

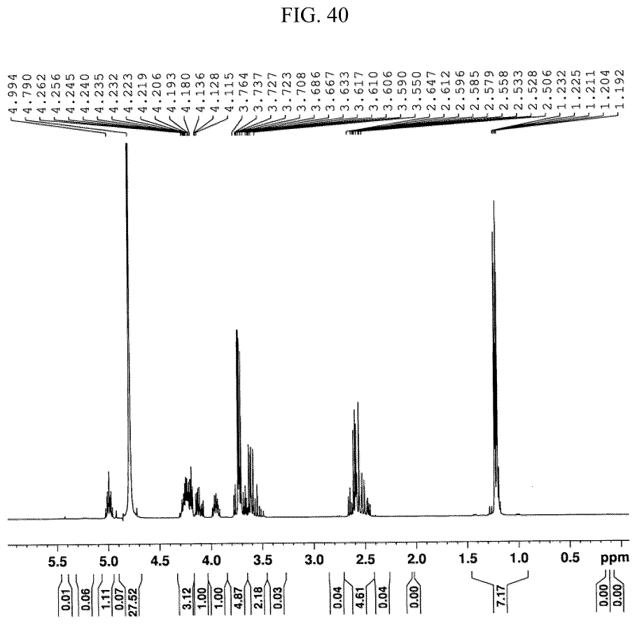

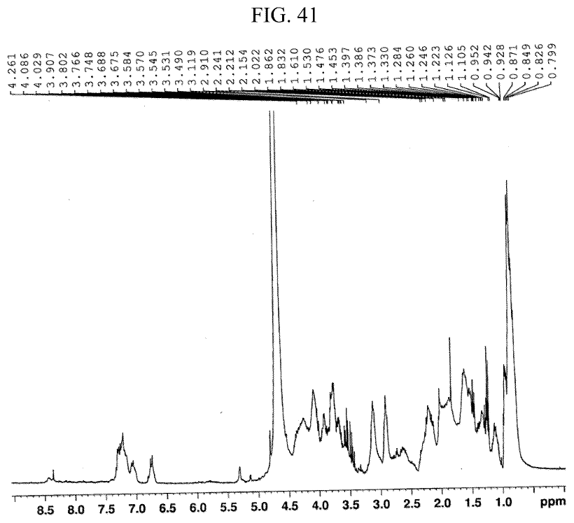

Disclosed herein are systems and methods for encapsulating a ketone molecule within a plant-derived protein crosslinked with a phytochemical. In some embodiments, the ketone molecule is bound to the plant-derived protein and phytochemical via hydrogen bonds as can be seen in FIG. 39.

The structure of the Ketone Molecule (KM) and the respective Fischer esterification scheme involving the dehydration of the parent carboxylic acid [(R)-3-hydroxybutyric acid] and the corresponding alcohol (glycerol) is shown below:

### Fischer Esterification Scheme

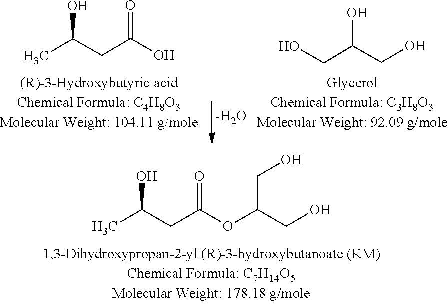

The parent alcohol glycerol can contain both primary and secondary alcohol groups. Various primary and secondary monoglycerides and diglycerides as well as the triglyceride reaction products are possible through the Fischer esterification scheme (below). The ester functionality has the possibility for conformational isomerism (cis/trans). Carboxylic acid (3-hydroxybutyric acid) is a chiral compound with two enantiomer (D and L) possibilities.

### Fischer Esterification Products:

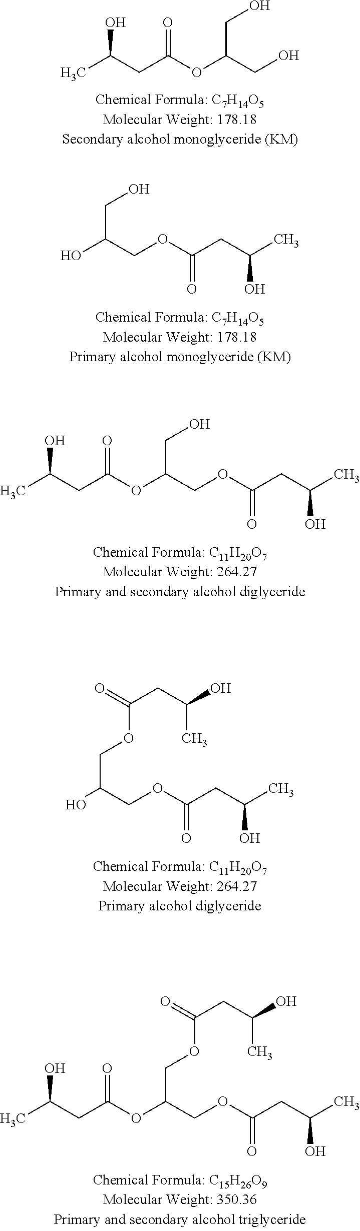

### Nicotinamide Riboside Molecules

Nicotinamide riboside (NR) is a precursor of nicotinamide adenine dinucleotide (NAD+). NAD+ is an essential coenzyme involved in various metabolic pathways, and an elevated NAD+ content in the body may provide health benefits.

Disclosed herein are systems and methods for encapsulating a NR molecule within a plant-derived protein crosslinked with a phytochemical.

The structure of NR and NR analogs are shown below:

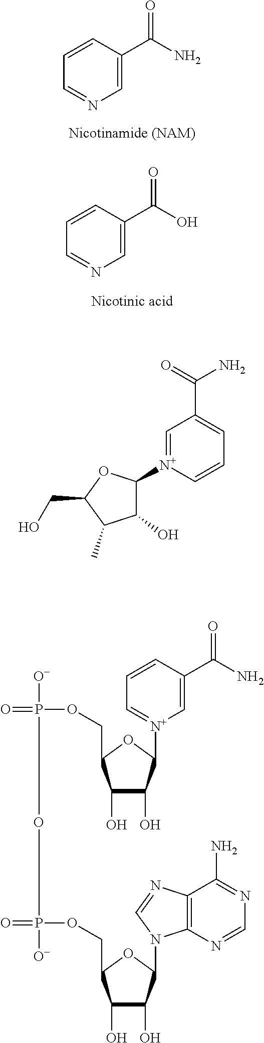

### Caffeine

Caffeine (CA) is a central nervous system (CNS) stimulant. It belongs to the methylxanthine class of purines. It increases alertness and attentional performance. Coffee bean is a major source of CA, and it tastes bitter. CA is classified by the US FDA as GRAS. Up to 400 mg of caffeine per day is safe. Toxic doses is over 10 grams per day for an adult.

Disclosed herein are systems and methods for encapsulating CA molecule within a plant-derived protein crosslinked with a phytochemical.

The structure of CA is shown below:

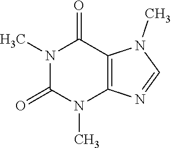

### Theacrine

Theacrine (TH) is a purine alkaloid, structurally similar to caffeine. It improves mood, energy and focus. Cupuaçu (tropical rainforest tree) and Chinese tea (kuchas) are major sources of TH. It affects adenosine signaling similar to caffeine. It is clinically safe and has non-habituating effects in humans. Daily use of up to 300 mg/day TH is safe.

Disclosed herein are systems and methods for encapsulating TH molecule within a plant-derived protein crosslinked with a phytochemical.

The structure of TH is shown below:

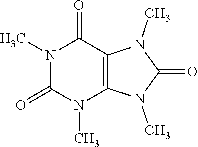 [SMILES: Cn1c(=O)c2c(n(C)c1=O)n(C)c(=O)n2C]

### Phytochemicals for Crosslinking Plant-Derived Protein:

Crosslinking agents may be beneficial for the synthesis of Ketone molecule (KM), NR, caffeine or theacrine encapsulated protein nanoparticles. Crosslinking agents crosslink the protein amino groups to form denser particles.

A common chemical crosslinker used for protein nanoparticle synthesis is glutaraldehyde. It is an effective crosslinking agent imparting nanoparticle stability and sustained drug release. Although effective, it may exert measurable systemic toxicity to humans especially when used for extended periods. As there no alternative non-toxic cross-linking agents, food, beverage, and pharmaceutical industries have continued to use glutaraldehyde in lower concentrations although the fear of long-term systemic toxicities with irreversible adverse toxic effects on human health persist.

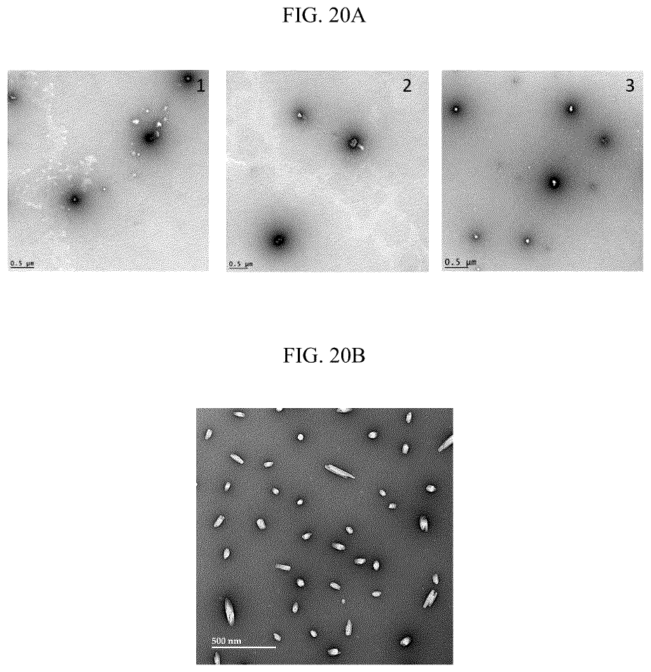

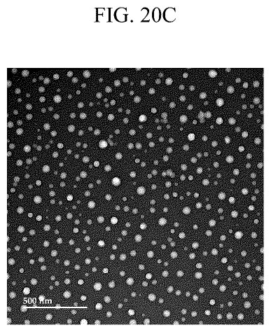

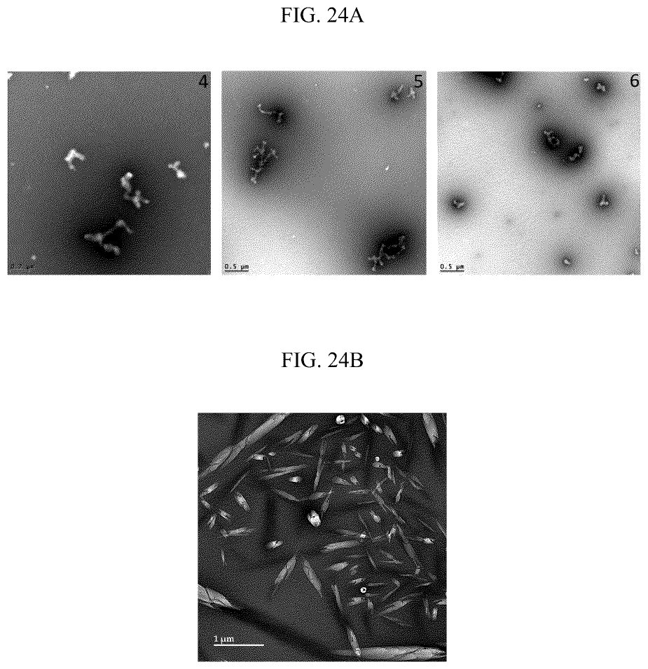

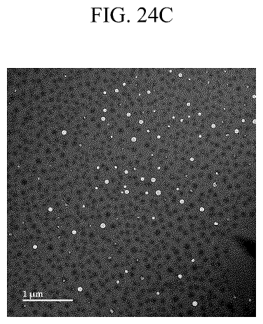

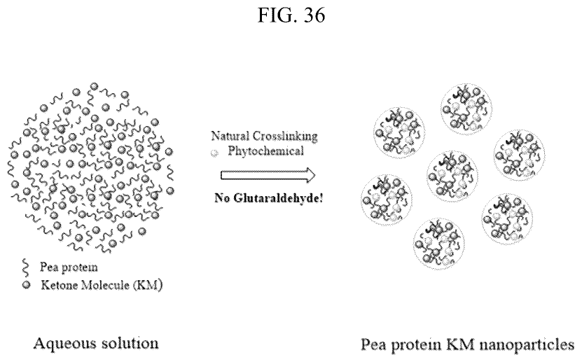

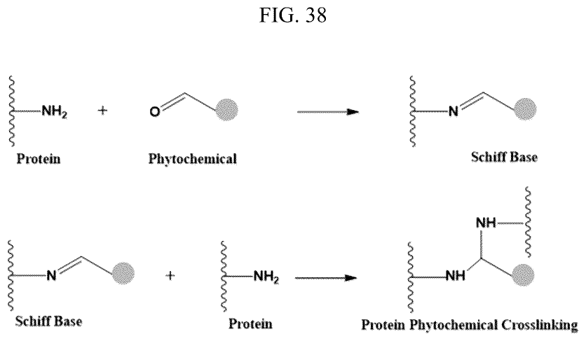

There is a need for biocompatible and non-toxic plant-based materials as effective and natural cross-linking agents for protein nanoparticle formulations (FIG. 38). Under oxidizing conditions, polyphenols, including mangiferin, epigallocatechin gallate (EGCG), quercetin and resveratrol, interact with amino groups of pea proteins to produce covalently bound crosslinks thus resulting in bioconjugations of various polyphenols with protein network. In this invention, as outlined in FIG. 36 the hydroxyl and oxo groups on the polyphenols can cross-link proteins. Plant-based materials including mangiferin (MGF), catechin, epigallocatechin gallate (EGCG), quercetin (QUE), and resveratrol (RESV) can be utilized for effective crosslinking and encapsulation of ketone molecules (KM) (FIG. 23), NR molecules (FIG. 24A), CA molecule (FIGS. 20B and 24B) and TH molecule (FIGS. 20C and 24C).

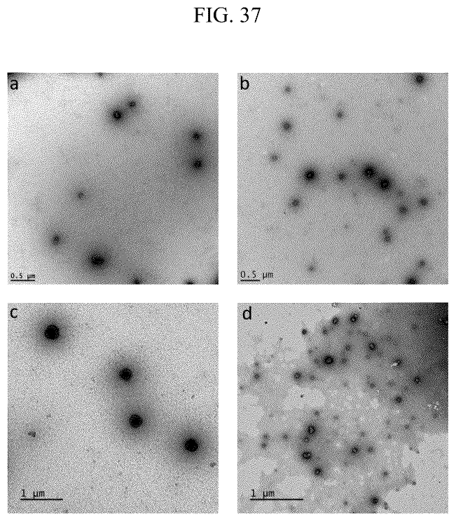

Disclosed herein are plant-based materials for effective crosslinking and encapsulation. Naturally available plant-based materials including mangiferin, EGCG, quercetin and resveratrol were utilized for effective protein crosslinking and encapsulation of KM (FIG. 37), NR, CA, and TH. Crosslinking agents can bind to the protein amino groups to form particles. The common chemical cross-linker used for protein nanoparticle synthesis is glutaraldehyde. Disclosed herein are methods and systems that cross-link plant derived protein without glutaraldehyde. Disclosed herein at methods and products that do not include glutaraldehyde.

Disclosed herein are biocompatible plant-based cross-linking agents including naturally available cross-linkers from tea (Epigallocatechin gallate; EGCG), mango (Mangiferin; MGF and various family of polyphenols and xanthonoids), berries (Quercetin; QUE), and grapes (Resveratrol; RESV).

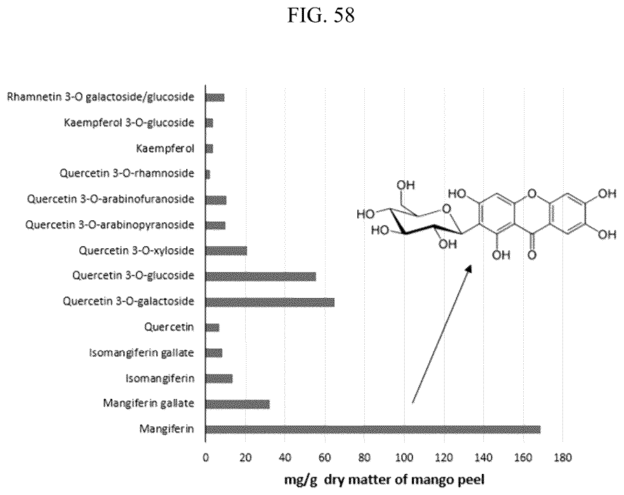

The predominant phytochemical of mango peel extract is mangiferin, as can be seen in FIG. 58. Mangiferin, a xanthonoid, is the glucoside of norathyriol and a natural phenolic compound. Mangiferin can be isolated from the leaves and bark of Mangifera indica (mango). It can also be extracted from mango peel (MP) and the kernel.

Catechin, a natural phenol, belongs to the flavonoid family. It is found in tea and fruits and is an antioxidant.

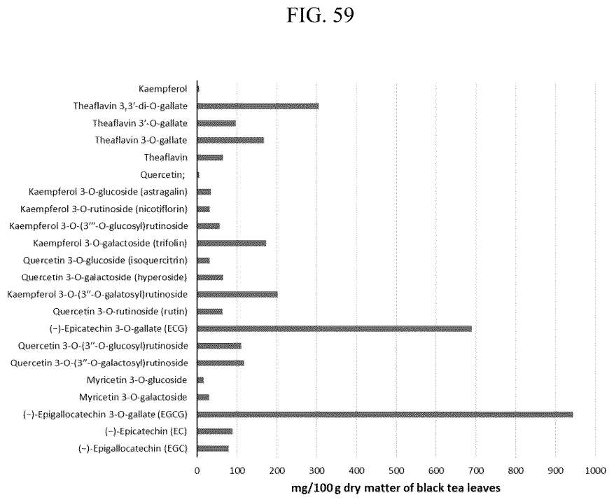

The predominant phytochemical of tea leaf extract is epigallocatechin gallate (EGCG) as can be seen in FIG. 59. EGCG is a catechin formed by the esterification of gallic acid and epigallocatechin. Polyphenols such as EGCG and catechin are found abundantly in tea leaves.

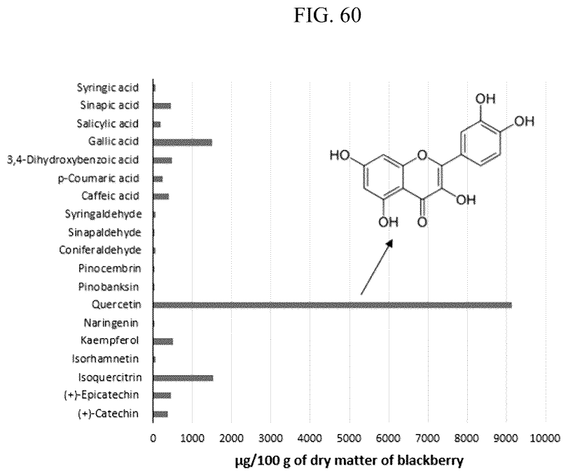

The predominant phytochemical of blackberry extract is quercetin, as can be seen in FIG. 60. Quercetin, a polyphenol belonging to the flavonoid group is widely found in fruits and vegetables. Quercetin is the most abundant polyphenol in black berry extract.

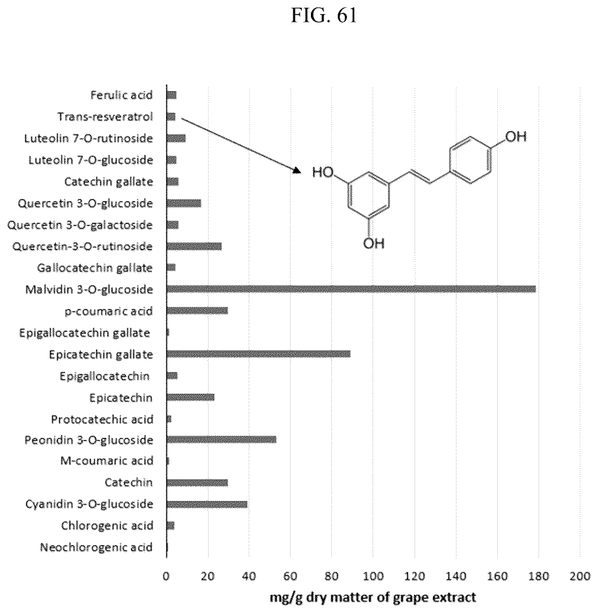

The predominant phytochemical of grape extract is trans-resveratrol, as can be seen in FIG. 61. Resveratrol, a stilbene-based plant phenol is found in grape extract. Resveratrol (RESV), is a stilbenoid, a form of natural phenol and a phytoalexin predominantly found in grape skin.

The highly acidic phenolic functional groups found in these plant-derived phytochemicals are effective for crosslinking with the amino groups in proteins. Creation of plant protein-polyphenol platforms, through interactions of polyphenolic compounds with protein amino side chains, are shown in FIG. 38.

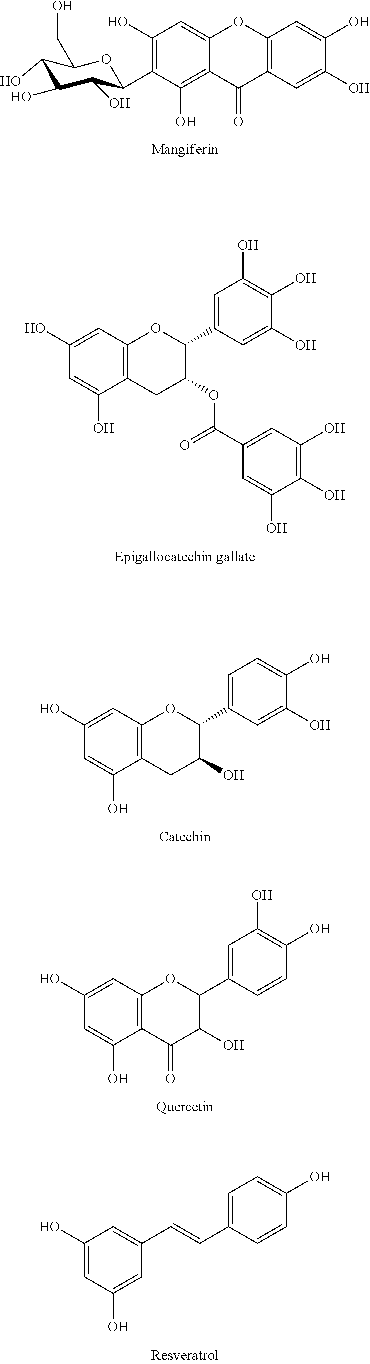

Interaction of the phytochemical with the water-soluble pea protein was studied using nuclear magnetic resonance (NMR) spectroscopy. Mangiferin, epigallocatechin gallate (EGCG), quercetin and resveratrol reactions were carried out individually with the pea protein to monitor for changes in the phytochemical and pea protein. NMR analysis shows distinct changes in phytochemical indicating covalent interaction with the protein (FIGS. 41-43, 45-46, 48-49, and 51-52). Then KM was added to the reaction mixture and further NMR analysis was carried out. In the presence of KM there is no change in the phytochemical or protein indicating the stability of KM in their presence as well (FIGS. 41-53). While using the plant extract (mango peel, tea, black berry, or grape extract) for the KM nanoparticle synthesis, it should be noted that the respective phytoextract is a phytochemical cocktail containing various other active ingredients which can also play a role in the crosslinking and encapsulation process whilst adding nutraceutical value to the formulation.

Under oxidizing conditions polyphenols react with protein amino groups (forming cross-links and creating a network). The reaction of ortho-quinones with proteins to form C—N or C—S bonds enable in the crosslinking process (FIG. 38). Plant proteins like pea and soy have plenty of lysine amino acid residues. These contain primary amino groups for crosslinking with phytochemicals. Plant based materials conjugates to the lysine residue's primary (ε-) amino group to generate a protein-polyphenol crosslinking platform. Sulphur containing amino acid cysteine is present in small amounts in pea and soy protein. Phytochemicals crosslink proteins by conjugating to the thiol group as well. Pea protein-mangiferin and pea protein-EGCG crosslinking products are shown below.

The structure of mangiferin crosslinked with pea protein can be seen on the left, below. The structure of EGCG crosslinked with pea protein can be seen on the right, below.

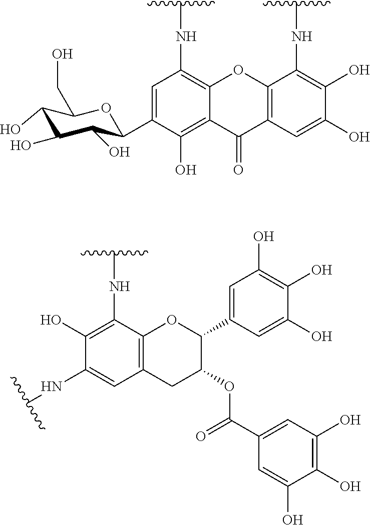

### Plant-Derived Protein:

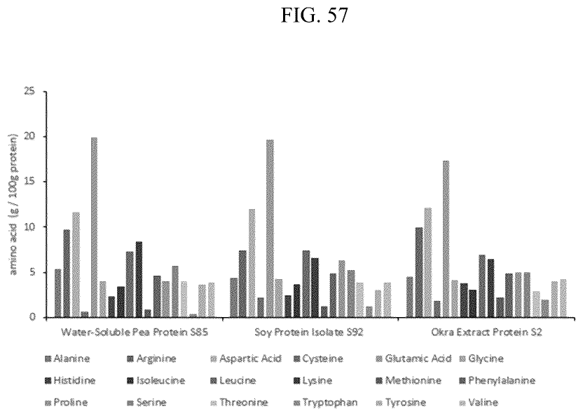

Proteins described herein can be derived from plants such as beans, okra, and pea. Bean protein can be soybean protein. Plant-derived protein can be water-soluble. Water-soluble protein can be hydrolyzed plant protein. FIG. 57 shows a comparison of the amino acid composition of pea protein, okra extract, and soy protein isolate.

TABLE 1
Comparison of the amino acid composition of
water-soluble Pea Protein (PP), Soy Protein
Isolate (SPI) and Okra Extract (OE) protein

| | Grams of amino acid per 100 grams of protein | | |
|---|---|---|---|
| | Water soluble | Soy Protein | Okra Extract |
| Amino acid | Pea Protein | Isolate | Protein |
| Alanine | 5.4 | 4.4 | 4.6 |
| Arginine | 9.8 | 7.4 | 10.0 |
| Aspartic Acid | 11.7 | 12.1 | 12.1 |
| Cysteine | 0.6 | 2.2 | 1.9 |
| Glutamic Acid | 19.9 | 19.7 | 17.4 |
| Glycine | 4.0 | 4.3 | 4.2 |
| Histidine | 2.4 | 2.5 | 3.8 |
| Isoleucine | 3.4 | 3.7 | 3.1 |
| Leucine | 7.3 | 7.5 | 7.0 |
| Lysine | 8.4 | 6.6 | 6.5 |
| Methionine | 0.9 | 1.2 | 2.2 |
| Phenylalanine | 4.6 | 4.9 | 4.9 |
| Proline | 4.0 | 6.4 | 5.0 |
| Serine | 5.7 | 5.2 | 5.0 |
| Threonine | 4.0 | 3.9 | 2.9 |
| Tryptophan | 0.4 | 1.3 | 2.0 |
| Tyrosine | 3.7 | 3.1 | 4.0 |
| Valine | 3.9 | 3.9 | 4.3 |

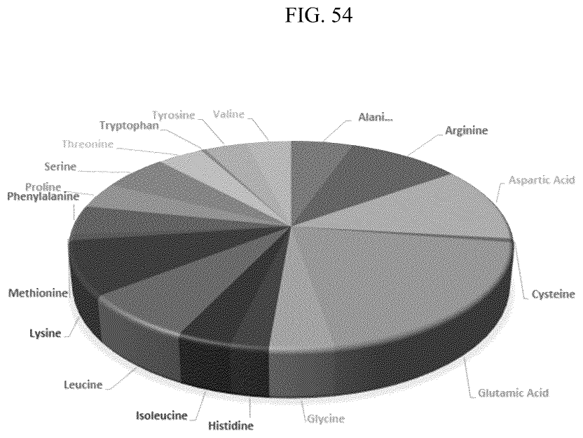

Water soluble pea protein can be derived from pea protein. Pea protein can be refined using enzyme digestion, filtration, or spray-drying. The protein content of pea protein can be >85%. Pea proteins can exist as globulins (65-80%) and can be composed of legumin, vicilin, and minor contributions from convicilin proteins. Pea protein can contain less than 7% moisture, less than 7% ash, and less than 7% crude fiber. Pea protein can have a pH between 5.0 and 6.0 when dissolved in water. The amino acid composition of pea protein can be found in FIG. 54.

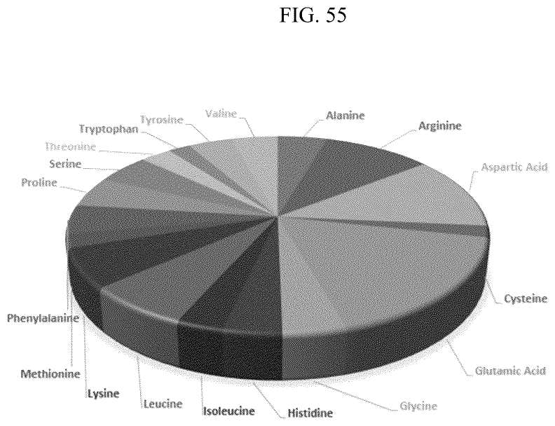

Okra Extract (Abelmoschus esculentus L.) can contain water (90%), protein (2%), carbohydrates (7%) and trace amounts of fat. Okra extract is a source of dietary fiber, vitamin C and vitamin K. Okra mucilage or gel from the pods is a natural polysaccharide and consists of D-galactose, L-rhamnose and L-galacturonic acid. Okra extract can be extracted in water and forms a gelatinous viscous solution. The amino acid composition of okra extract can be found in FIG. 55.

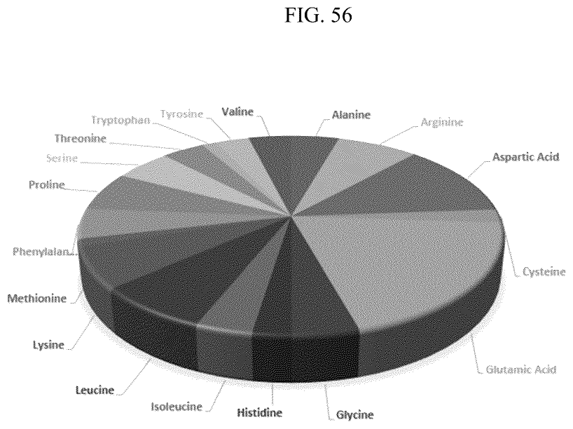

Soy Protein Isolate (SPI) is derived from soybean. Soy protein isolate can be composed of β-conglycinin, glycinin, and lipophilic proteins. Soy protein can have a protein content of 92% (in dry basis). Additionally, it can contain moisture 6%, Ash 4.1%, Fat (PE extract) 0.8%, crude fiber (crude) 0.25%, calcium 0.15%, phosphorus 0.8%, sodium 1.3%, potassium 0.05%, and can display a pH (water slurry) of 7.1. The amino acid composition of soy protein isolate can be found in FIG. 56.

### Kits

A kit may include one or more containers housing one or more of the components provided in this disclosure and instructions for use. Specifically, such kits may include one or more compositions described herein, along with instructions describing the intended application and the proper use and/or disposition of these compositions. Kits may contain the components in appropriate concentrations or quantities for running various experiments.

### Methods of Manufacturing Nanoparticles

Disclosed herein are systems and methods of producing nanoparticles comprising plant-derived protein crosslinked with one or more phytochemicals as described herein. The nanoparticles can encapsulate one or more small molecules, such as ketone molecules, nicotinamide riboside molecules, caffeine molecules and/or theacrine molecules. In some embodiments, plant-derived protein, phytochemical containing powder, and ketone molecules are combined in water. In some embodiments, plant-derived protein, phytochemical containing powder, and NR molecules are combined in water. In some embodiments, plant-derived protein, phytochemical containing powder, and caffeine molecules are combined in water. In some embodiments, plant-derived protein, phytochemical containing powder, and theacrine molecules are combined in water.

In some embodiments, ethanol is added to a mixture of plant-derived protein, phytochemical containing powder, and ketone molecules. In some embodiments, ethanol is added to a mixture of plant-derived protein, phytochemical containing powder, and NR molecules. In some embodiments, the ethanol is added to the mixture at a 1:10 to 1:1 ratio by volume of ethanol to mixture. In some embodiments, the volume ratio of ethanol to mixture is greater than or equal to: 1:10, 1:9, 1:8, 1:7, 1:6, 1:5, 1:4, 1:3, 1:2, or 1:1. In some embodiments, the volume ratio of ethanol to mixture is less than or equal to 1:1, 1:2, 1:3, 1:4, 1:5, 1:6, 1:7, 1:8, 1:9, or 1:10. In some embodiments, the volume ratio of ethanol to mixture is between 1:4 and 1:7, 1:5 and 1:9, or 1:6 and 1:10. In some embodiments, the volume ratio of ethanol to mixture is less than or equal to 1:7. In some embodiments, the ethanol is removed from the mixture of plant-derived protein, phytochemical containing powder, and ketone molecules. In some embodiments, the ethanol is removed from the mixture of plant-derived protein, phytochemical containing powder, and NR molecules. In some embodiments, the ethanol is removed from the mixture by incubating the mixture at a temperature greater than or equal to 37° C. In some embodiments the ethanol is removed by rotovapor evaporation.

In some embodiments, the phytochemical containing powder comprises tea leaves, blackberry, grape, mango, or okra. In some embodiments, the phytochemical containing powder comprises loose-leaf tea, black berry powder, grapes, mango peel powder, or okra extract powder. In some embodiments, the concentration of phytochemical in the phytochemical containing powder is greater than or equal to 90 μg/g dry matter, 1 mg/g dry matter, 170 mg/g dry matter, or 950 mg/g dry matter.

In some embodiments, the phytochemical containing powder is mango peel powder. In some embodiments, the phytochemical of mango peel powder is mangiferin. In some embodiments the concentration of phytochemical in mango peel powder is less than or equal to 200 mg/g dry matter, 190 mg/g dry matter, 180 mg/g dry matter, 170 mg/g dry matter, 160 mg/g dry matter, 150 mg/g dry matter, 140 mg/g dry matter, 130 mg/g dry matter, 120 mg/g dry matter, 110 mg/g dry matter, or 100 mg/g dry matter. In some embodiments, the concentration of phytochemical in mango peel powder is greater than or equal to 100 mg/g dry matter, 110 mg/g dry matter, 120 mg/g dry matter, 130 mg/g dry matter, 140 mg/g dry matter, 150 mg/g dry matter, 160 mg/g dry matter, 170 mg/g dry matter, 180 mg/g dry matter, 190 mg/g dry matter, or 200 mg/g dry matter. In some embodiments, the concentration of phytochemical in mango peel powder is between 100 mg/g to 120 mg/g dry matter, between 110 mg/g to 130 mg/g dry matter, between 120 mg/g to 140 mg/g dry matter, between 150 mg/g to 170 mg/g dry matter, between 160 mg/g to 180 mg/g dry matter, between 170 mg/g to 190 mg/g dry matter, or between 180 mg/g to 200 mg/g dry matter.

In some embodiments, the weight ratio of mangiferin to plant-derived protein is greater than or equal to 1:60, 1:55, 1:50, 1:45, 1:40, 1:35, or 1:30. In some embodiments, the weight ratio of mangiferin to plant-derived protein is less than or equal to 1:30, 1:35, 1:40, 1:45, 1:50, 1:55, or 1:60. In some embodiments, the weight ratio of mangiferin to plant-derived protein is between 1:30 and 1:40, between 1:35 and 1:45, between 1:40 and 1:50, between 1:45 and 1:55, or between 1:50 and 1:60. In some embodiments, the weight ratio of mangiferin to plant-derived protein is 1:40.

In some embodiments, the phytochemical containing powder comprises tea leaves. In some embodiments, the phytochemical of tea leaves is Epigallocatechin 3-O-gallate (EGCG). In some embodiments the concentration of phytochemical in tea leaves is less than or equal to 1050 mg/g dry matter, 1000 mg/g dry matter, 950 mg/g dry matter, 900 mg/g dry matter, 850 mg/g dry matter, 800 mg/g dry matter, 750 mg/g dry matter, 700 mg/g dry matter, 650 mg/g dry matter, 600 mg/g dry matter, 550 mg/g dry matter, or 500 mg/g dry matter. In some embodiments, the concentration of phytochemical in tea leaves is greater than or equal to 500 mg/g dry matter, 550 mg/g dry matter, 600 mg/g dry matter, 650 mg/g dry matter, 700 mg/g dry matter, 750 mg/g dry matter, 800 mg/g dry matter, 850 mg/g dry matter, 900 mg/g dry matter, 950 mg/g dry matter, 1000 mg/g dry matter, or 1050 mg/g dry matter. In some embodiments, the concentration of phytochemical in tea leaves is between 500 mg/g to 600 mg/g dry matter, between 550 mg/g to 650 mg/g dry matter, between 600 mg/g to 700 mg/g dry matter, between 650 mg/g to 750 mg/g dry matter, between 700 mg/g to 800 mg/g dry matter, between 750 mg/g to 850 mg/g dry matter, between 800 mg/g to 900 mg/g dry matter, between 850 mg/g to 950 mg/g dry matter, between 900 mg/g to 1000 mg/g dry matter or between 950 mg/g to 1050 mg/g dry matter.

In some embodiments, the weight ratio of EGCG to plant-derived protein is greater than or equal to 1:500, 1:450, 1:400, 1:350, 1:300, 1:250, 1:200, 1:150, or 1:100. In some embodiments, the weight ratio of EGCG to plant-derived protein is less than or equal to 1:100, 1:150, 1:200, 1:250, 1:300, 1:350, 1:400, 1:450, or 1:500. In some embodiments, the weight ratio of EGCG to plant-derived protein is between 1:100 and 1:200, between 1:150 and 1:250, between 1:200 and 1:300, between 1:250 and 1:350, between 1:300 and 1:400, between 1:350 and 1:450, or between 1:400 and 1:500. In some embodiments, the weight ratio of EGCG to plant-derived protein is 1:265.

In some embodiments, the phytochemical containing powder comprises grape extract. In some embodiments, the phytochemical of grape extract is trans-resveratrol In some embodiments the concentration of phytochemical in grape extract is less than or equal to 40 mg/g dry matter, 35 mg/g dry matter, 30 mg/g dry matter, 25 mg/g dry matter, 20 mg/g dry matter, 19 mg/g dry matter, 18 mg/g dry matter, 17 mg/g dry matter, 16 mg/g dry matter, 15 mg/g dry matter, 14 mg/g dry matter, 13 mg/g dry matter, 12 mg/g dry matter, 11 mg/g dry matter, 10 mg/g dry matter, 9 mg/g dry matter, 8 mg/g dry matter, 7 mg/g dry matter, 6 mg/g dry matter, 5 mg/g dry matter, 4 mg/g dry matter, 3 mg/g dry matter, 2 mg/g dry matter, or 1 mg/g dry matter. In some embodiments, the concentration of phytochemical in grape extract is greater than or equal to 1 mg/g dry matter, 2 mg/g dry matter, 3 mg/g dry matter, 4 mg/g dry matter, 5 mg/g dry matter, 6 mg/g dry matter, 7 mg/g dry matter, 8 mg/g dry matter, 9 mg/g dry matter, 10 mg/g dry matter, 11 mg/g dry matter, 12 mg/g dry matter, 13 mg/g dry matter, 14 mg/g dry matter, 15 mg/g dry matter, 16 mg/g dry matter, 17 mg/g dry matter, 18 mg/g dry matter, 19 mg/g dry matter, 20 mg/g dry matter, 25 mg/g dry matter, 30 mg/g dry matter, 35 mg/g dry matter, or 40 mg/g dry matter. In some embodiments, the concentration of phytochemical in grape extract is between 1 mg/g to 3 mg/g dry matter, between 2 mg/g to 4 mg/g dry matter, between 5 mg/g to 7 mg/g dry matter, between 6 mg/g to 8 mg/g dry matter, between 7 mg/g to 9 mg/g dry matter, between 8 mg/g to 10 mg/g dry matter, between 9 mg/g to 11 mg/g dry matter, between 10 mg/g to 12 mg/g dry matter, between 11 mg/g to 13 mg/g dry matter, between 12 mg/g to 14 mg/g dry matter, between 13 mg/g to 15 mg/g dry matter, between 14 mg/g to 16 mg/g dry matter, between 15 mg/g to 17 mg/g dry matter, between 16 mg/g to 18 mg/g dry matter, between 17 mg/g to 19 mg/g dry matter, between 18 mg/g to 20 mg/g dry matter, between 19 mg/g to 25 mg/g dry matter, between 20 mg/g to 30 mg/g dry matter, between 25 mg/g to 35 mg/g dry matter, or between 30 mg/g to 40 mg/g dry matter.

In some embodiments, the weight ratio of resveratrol to plant-derived protein is greater than or equal to 1:500, 1:450, 1:400, 1:350, or 1:300. In some embodiments, the weight ratio of resveratrol to plant-derived protein is less than or equal to 1:300, 1:350, 1:400, 1:450, or 1:500. In some embodiments, the weight ratio of resveratrol to plant-derived protein is between 1:300 and 1:400, between 1:350 and 1:450, or between 1:400 and 1:500. In some embodiments, the weight ratio of resveratrol to plant-derived protein is 1:400.

In some embodiments, the phytochemical containing powder comprises blackberry. In some embodiments, the phytochemical of blackberry is quercetin. In some embodiments the concentration of phytochemical in blackberry is less than or equal to 10000 μg/100 g dry matter, 95000 μg/100 g dry matter, 90000 μg/100 g dry matter, 85000 μg/100 g dry matter, 80000 μg/100 g dry matter, 75000 μg/100 g dry matter, 70000 μg/100 g dry matter, 65000 μg/100 g dry matter, 60000 μg/100 g dry matter, 55000 μg/100 g dry matter, or 50000 μg/100 g dry matter. In some embodiments, the concentration of phytochemical in blackberry is greater than or equal to 50000 μg/100 g dry matter, 55000 μg/100 g dry matter, 60000 μg/100 g dry matter, 65000 μg/100 g dry matter, 70000 μg/100 g dry matter, 75000 μg/100 g dry matter, 80000 μg/100 g dry matter, 85000 μg/100 g dry matter, 90000 μg/100 g dry matter, 95000 μg/100 g dry matter, or 100000 μg/100 g dry matter. In some embodiments, the concentration of phytochemical in blackberry is between 50000 μg/100 g to 60000 μg/100 g dry matter, between 55000 μg/100 g to 65000 μg/100 g dry matter, between 60000 μg/100 g to 70000 μg/100 g dry matter, between 65000 μg/100 g to 75000 μg/100 g dry matter, between 70000 μg/100 g to 80000 μg/100 g dry matter, between 75000 μg/100 g to 85000 μg/100 g dry matter, between 80000 μg/100 g to 90000 μg/100 g dry matter, between 85000 μg/100 g to 95000 μg/100 g dry matter, or between 90000 μg/100 g to 100000 μg/100 g dry matter.

In some embodiments, the weight ratio of quercetin to plant-derived protein is greater than or equal to 1:40000, 1:35000, 1:30000, 1:25000, 1:24000, 1:23500, 1:23,000, 1:22,750, 1:22,500, 1:22,250, or 1:22,000. In some embodiments, the weight ratio of quercetin to plant-derived protein is less than or equal to 1:22,000, 1:22,250, 1:22,500, 1:22, 750, 1:23,000, 1:23500, 1:24000, 1:25000, 1:30000, or 1:40000. In some embodiments, the weight ratio of quercetin to plant-derived protein is between 1:22,000 and 1:22,500, between 1:22,250 and 1:22,750, between 1:22,500 and 1:23,000, between 1:22,750 and 1:23,500, between 1:23,000 and 1:24,000, between 1:23,500 and 1:25,000, between 1:24,000 and 1:30,000, or between 1:25,000 and 1:40,000. In some embodiments, the weight ratio of quercetin to plant-derived protein is 1:22,222.

In some embodiments the water has undergone one or more processes such as distillation or filtration. In some embodiments the water is distilled water.

In some embodiments, the dry weight ratio of ketone molecules to plant-derived protein is less than or equal to 1:1, 1:2, 1:3, 1:4, 1:5, 1:6, 1:7, 1:8, 1:9, or 1:10. In some embodiments, the dry weight ratio of ketone molecules to plant-derived protein is greater than or equal to 1:10, 1:9, 1:8, 1:7, 1:6, 1:5, 1:4, 1:3, 1:2, or 1:1. In some embodiments, the dry weight ratio of ketone molecule to plant-derived protein is between 1:10 and 1:1, between 1:5 and 1:2, or between 1:3 and 1:2. In some embodiments, the dry weight ratio of ketone molecule to plant-derived protein is less than or equal to 1:2. In some embodiments, the dry weight ratio of ketone molecule to plant-derived protein is 1:2.5.

In some embodiments, the dry weight ratio of NR molecules to plant-derived protein is less than or equal to 1:1, 1:2, 1:3, 1:4, 1:5, 1:6, 1:7, 1:8, 1:9, or 1:10. In some embodiments, the dry weight ratio of NR molecules to plant-derived protein is greater than or equal to 1:10, 1:9, 1:8, 1:7, 1:6, 1:5, 1:4, 1:3, 1:2, or 1:1. In some embodiments, the dry weight ratio of NR molecule to plant-derived protein is between 1:10 and 1:1, between 1:5 and 1:2, or between 1:3 and 1:2. In some embodiments, the dry weight ratio of NR molecule to plant-derived protein is less than or equal to 1:2. In some embodiments, the dry weight ratio of NR molecule to plant-derived protein is 1:3.2.

In some embodiments, the dry weight ratio of caffeine molecules to plant-derived protein is less than or equal to 1:1, 1:2, 1:3, 1:4, 1:5, 1:6, 1:7, 1:8, 1:9, or 1:10. In some embodiments, the dry weight ratio of caffeine molecules to plant-derived protein is greater than or equal to 1:10, 1:9, 1:8, 1:7, 1:6, 1:5, 1:4, 1:3, 1:2, or 1:1. In some embodiments, the dry weight ratio of caffeine molecule to plant-derived protein is between 1:10 and 1:1, between 1:5 and 1:2, or between 1:3 and 1:2. In some embodiments, the dry weight ratio of caffeine molecule to plant-derived protein is less than or equal to 1:2. In some embodiments, the dry weight ratio of caffeine molecule to plant-derived protein is 1:2.

In some embodiments, the dry weight ratio of TH molecules to plant-derived protein is less than or equal to 1:1, 1:2, 1:3, 1:4, 1:5, 1:6, 1:7, 1:8, 1:9, or 1:10. In some embodiments, the dry weight ratio of TH molecules to plant-derived protein is greater than or equal to 1:10, 1:9, 1:8, 1:7, 1:6, 1:5, 1:4, 1:3, 1:2, or 1:1. In some embodiments, the dry weight ratio of TH molecule to plant-derived protein is between 1:10 and 1:1, between 1:5 and 1:2, or between 1:3 and 1:2. In some embodiments, the dry weight ratio of TH molecule to plant-derived protein is less than or equal to 1:2. In some embodiments, the dry weight ratio of TH molecule to plant-derived protein is 1:2.

In some embodiments, the weight ratio of plant-derived protein to water is greater than or equal to: 1:100, 1:90, 1:80, 1:70, 1:60, 1:50, 1:40, 1:30, 1:20, or 1:10. In some embodiments, the weight ratio of plant-derived protein to water is less than or equal to 1:10, 1:20, 1:30, 1:40, 1:50, 1:60, 1:70, 1:80, 1:90, or 1:100. In some embodiments, the weight ratio of plant-derived protein to water is between 1:40 and 1:70, 1:50 and 1:90, or 1:60 and 1:100. In some embodiments, the weight ratio of plant-derived protein to water is less than or equal to 1:70. In some embodiments, the weight ratio of plant-derived protein to water is 1:66.7. In some embodiments, the weight ratio of plant-derived protein to water is 1:20.

### Nanoparticles

Disclosed herein are nanoparticles comprising plant-derived protein crosslinked with one or more phytochemicals. In some embodiments, a nanoparticle can comprise one or more ketone molecules.

In some embodiments, the average diameter of a nanoparticle is less than or equal to 700 nm, 600 nm, 500 nm, 400 nm, 300 nm, 200 nm, 100 nm, 90 nm, 80 nm, 70 nm, 60 nm, or 50 nm. In some embodiments, the average diameter of a nanoparticle is greater than or equal to 50 nm, 60 nm, 70 nm, 80 nm, 90 nm, 100 nm, 200 nm, 300 nm, 400 nm, 500 nm, 600 nm, or 700 nm. In some embodiments, the average diameter of a nanoparticle is between 50 nm and 70 nm, between 60 nm and 80 nm, between 70 nm and 90 nm, between 80 nm and 100 nm, between 90 nm and 200 nm, between 100 nm and 300 nm, between 100 nm and 400 nm, between 100 nm and 500 nm, between 200 nm and 400 nm, between 200 nm and 50 nm, between 300 nm and 500 nm, between 400 nm and 600 nm, or between 500 nm and 700 nm.

In some embodiments, the nanoparticles comprise phenols. In some embodiments, the phenol content of a nanoparticle is quantified using total gallic acid equivalents (GAE). In some embodiments, the phenol content of nanoparticles described herein is greater than 5 mg GAE/g, 10 mg GAE/g, 20 mg GAE/g, 30 mg GAE/g, 40 mg GAE/g, 50 mg GAE/g, 60 mg GAE/g, 70 mg GAE/g, 80 mg GAE/g, 90 mg GAE/g, 100 mg GAE/g, 150 mg GAE/g, 200 mg GAE/g, 250 mg GAE/g, 300 mg GAE/g, or 350 mg GAE/g. In some embodiments, the phenol content of nanoparticles described herein is less than 400 mg GAE/g, 350 mg GAE/g, 300 mg GAE/g, 250 mg GAE/g, 200 mg GAE/g, 150 mg GAE/g, 100 mg GAE/g, 90 mg GAE/g, 80 mg GAE/g, 70 mg GAE/g, 60 mg GAE/g, 50 mg GAE/g, 40 mg GAE/g, 30 mg GAE/g, 20 mg GAE/g, 10 mg GAE/g, or 5 mg GAE/g. In some embodiments, the phenol content of nanoparticles described herein is between 5 mg GAE/g and 20 mg GAE/g, between 10 mg GAE/g and 30 mg GAE/g, between 20 mg GAE/g and 40 mg GAE/g, between 30 mg GAE/g and 50 mg GAE/g, between 40 mg GAE/g and 60 mg GAE/g, between 50 mg GAE/g and 70 mg GAE/g, between 60 mg GAE/g and 80 mg GAE/g, between 70 mg GAE/g and 90 mg GAE/g, between 80 mg GAE/g and 100 mg GAE/g, between 90 mg GAE/g and 150 mg GAE/g, between 100 mg GAE/g and 200 mg GAE/g, between 150 mg GAE/g and 250 mg GAE/g, between 200 mg GAE/g and 300 mg GAE/g, between 250 mg GAE/g and 350 mg GAE/g, or between 300 mg GAE/g and 400 mg GAE/g.

### EXAMPLES

### Example 1—Ketone Molecule Synthesis

Ketone molecules as described herein were produced through a Fisher esterification reaction of (R)-3-hydroxybutyric acid and glycerol, as described herein, resulting in a mixture of Compounds II-V, as described herein. Compound II was purified by fractional distillation according to methods known in the art. (See, e.g., www.sciencedirect.com/topics/chemistry/fractional-distillation) The resulting ketone molecules comprised a molar ratio of Compound II to Compounds II-V of greater than 80:20.

### Example 2a—Nicotinamide Riboside Molecule Synthesis

Nicotinamide riboside molecules and Compound IV were obtained from Thorne Healthcare.

### Example 2b—Caffeine Molecule Synthesis

Caffeine molecule was obtained from Sigma-Aldrich.

### Example 2c—Theacrine Molecule Synthesis

Theacrine molecule was obtained from BulkStimulants.com.

### Example 3—Ketone Molecule (KM) Characterization

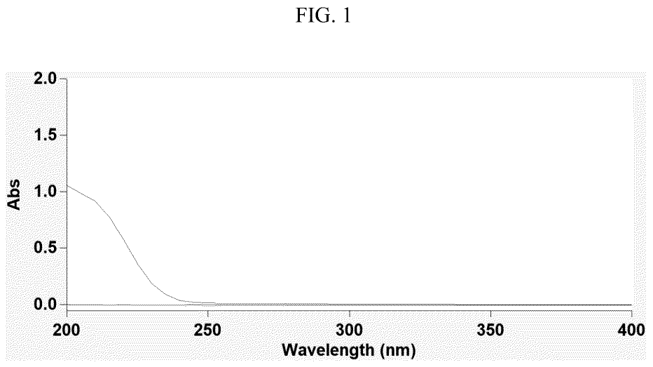

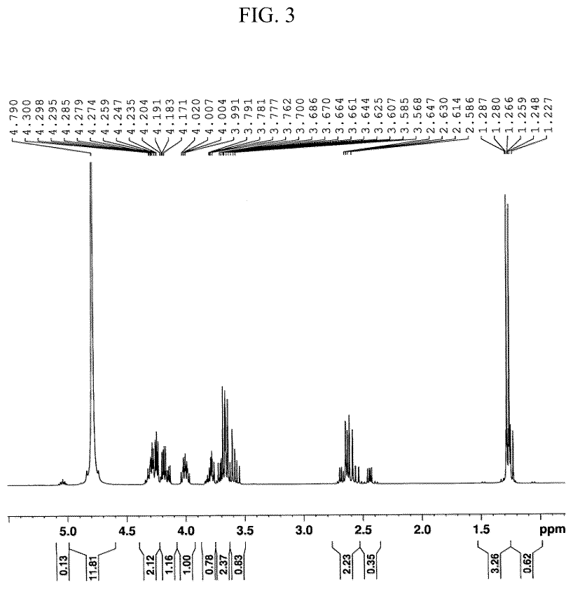

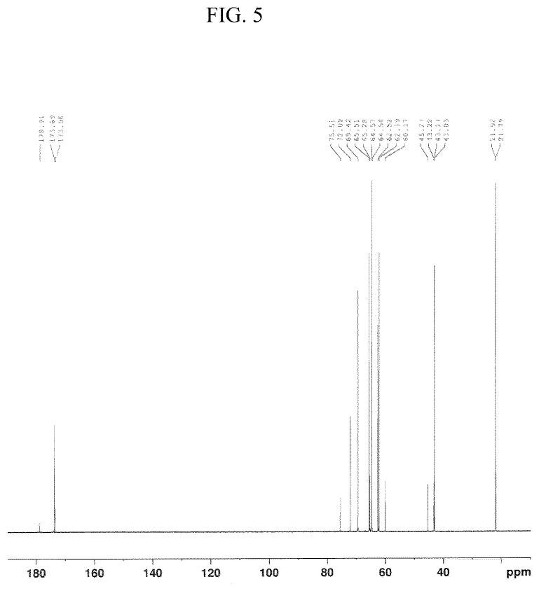

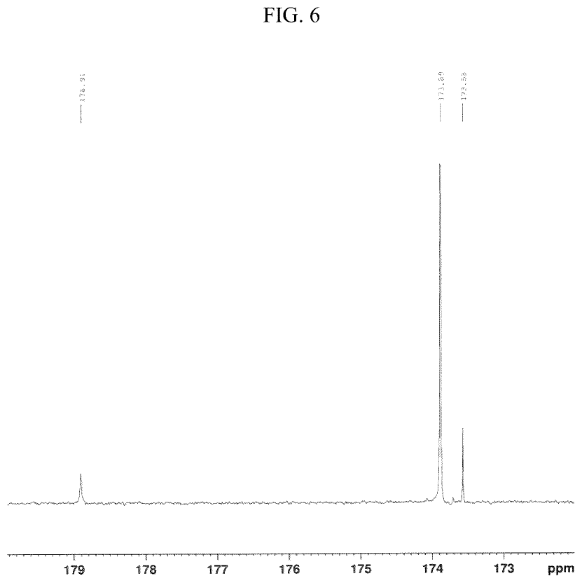

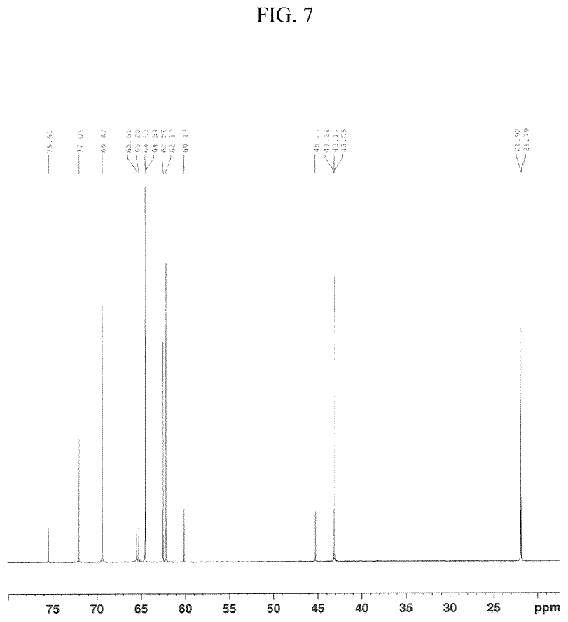

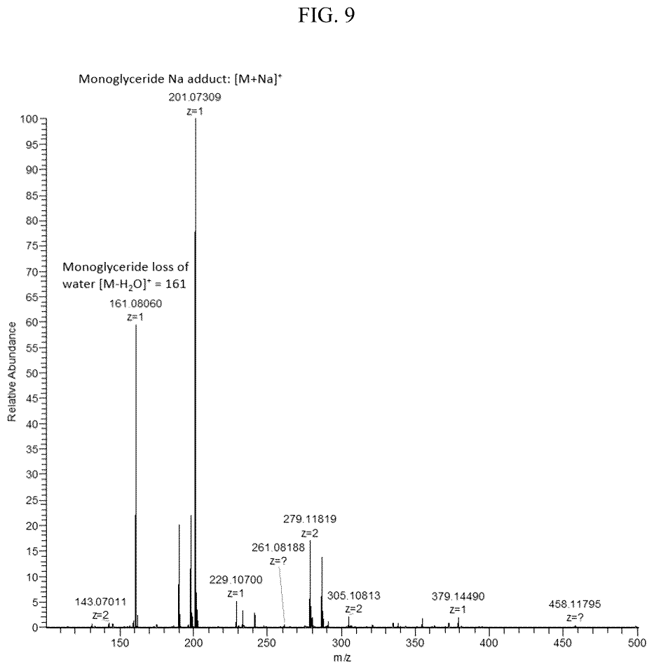

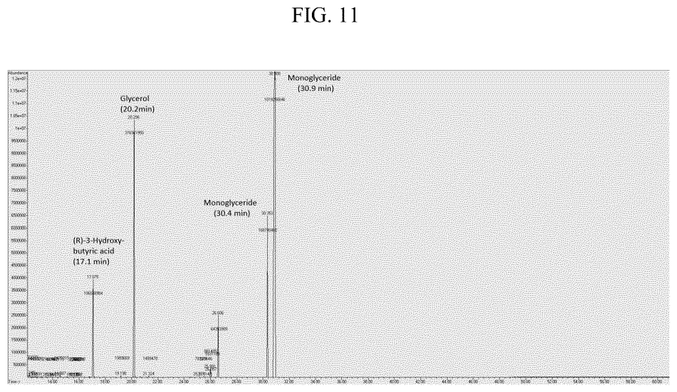

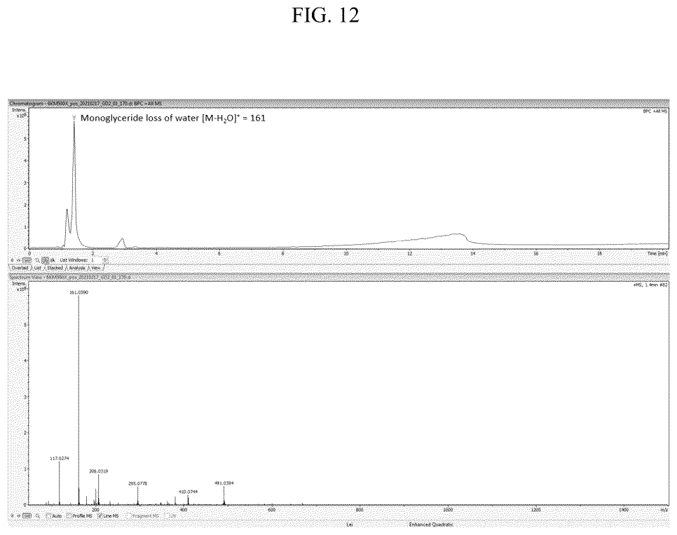

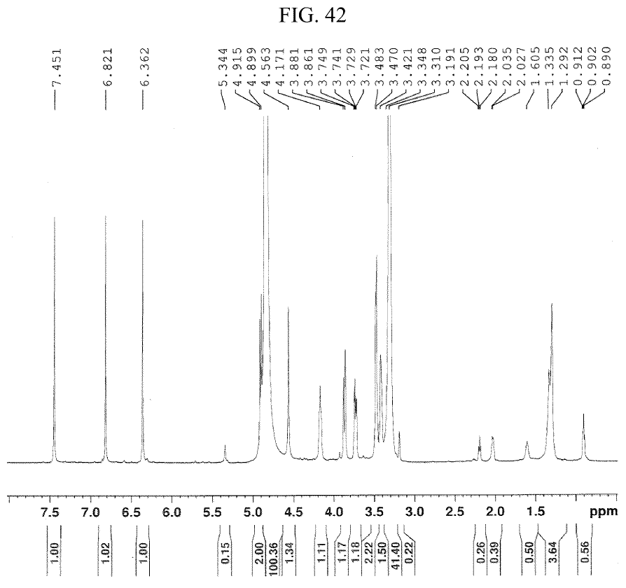

Appearance: KM was a faintly cloudy oily highly viscous liquid.
Smell: KM's smell was neutral.
Solubility: KM was water soluble and was miscible with ethanol. KM was not soluble in
methylene chloride.
pH: KM displayed a pH of 4.5 in water indicating an acidic aqueous solution.
UV-visible absorption spectroscopy: The electronic transition in KM pertains to the ultraviolet region (UV) based on the UV-visible absorption measurement. KM showed a broad absorption until ˜245 nm in the UV region (FIG. 1). There was no chromophoric characteristic absorption peaks in the visible region. Therefore, with regards to tracking KM or KM encapsulated protein nanoparticles, UV-visible spectroscopy was not utilized.
Nuclear magnetic resonance spectroscopy: 1H nuclear magnetic resonance (NMR) spectra was recorded at 300 MHz on a Bruker 300 MHz spectrometer. ¹³C NMR spectra was recorded at 151 MHz on a Bruker 600 MHz spectrometer. NMR spectra was recorded in deuterated water (D2O). The 1H chemical shifts were reported relative to internal D2O (FIG. 3). The ¹³C NMR chemical shifts were reported relative to an external tetramethylsilane (TMS) standard (FIG. 5-7). KM is a carboxylate ester comprising of the parent carboxylic acid (3-hydroxybutyric acid) and the corresponding alcohol (glycerol). Various glycerides are possible products during KM synthesis. The NMR showed ˜77% of the KM, namely the monoglyceride. In the NMR, starting materials glycerol (11%) and 3-hydroxybutyric acid (12%) were also seen. FIG. 42 showed a ¹H NMR of KM in D₂O.
Mass Spectrometry: A range of sophisticated mass spectrometric techniques had been utilized to develop a reliable quality control parameters for the Ketone Molecule (KM) and its protein encapsulated products. Analysis of KM using electrospray ionization-mass spectrometry (ESI-MS) technique had been carried out. Apart from obtaining the elemental/isotopic signature of KM using MS, the [M+H]+ peak had been used for molecular weight estimation. Based on the chemical structure, the calculated molecular weight of KM was 178.18 g/mole. The [M+Na]+ peak of 201.07 was detected as the major peak indicating the presence of monoglyceride (KM) (FIG. 9).
Gas chromatography-mass spectrometry (GC-MS) and Liquid chromatography-mass spectrometry (LC-MS): In addition to the mass spectrometric analysis, gas chromatography-mass spectrometry (GC-MS) and liquid chromatography-mass spectrometry (LC-MS) was utilized for the quality control confirmation of KM. GC-MS and LC-MS techniques are excellent analytical and quality control tools in detecting KM purity and the presence of starting materials. KM is a carboxylate ester comprising of the parent carboxylic acid (3-hydroxybutyric acid) and the corresponding alcohol (glycerol). In the GC-MS analysis (FIG. 11) 3-hydroxybutyric acid (˜10%) and glycerol (˜23%) were seen at 17.1 min and 20.2 min retention RT respectively. The two monoglyceride peaks were seen at 30.4 min and 30.9 min retention time (RT). The peak at 30.4 min (˜16%) and the peak at 30.9 min (˜50%) gave a combined KM monoglyceride percentage of ˜66%. There were no diglycerides and triglyceride peaks (should be seen after ˜30 min RT). In the LC-MS analysis (FIG. 12), the predominant peak corresponded to the loss of water from the monoglyceride. The predominant peak at 1.4 min RT corresponded to the [M−H2O]+ peak of 161—corroborating results from NMR as well as results from GC-MS.
Circular Dichroism (CD): Chiral molecules are mirror images or enantiomers. The physical properties of enantiomers are identical in every way except with regards to how the molecules interact with polarized light and how they interact with other chiral molecules. Enantiomerism is a special form of isomerism. Circular dichroism (CD), measured as a function of wavelength, is the difference in absorbance of left-handed circularly polarized light (L-CPL) and right-handed circularly polarized light (R-CPL). When chiral chromophores are present, one state of circularly polarized light is absorbed to a greater or lesser extent than the other. Over corresponding wavelengths, a circular dichroism signal can, therefore, be positive or negative, depending on whether L-CPL is absorbed to a greater extent than R-CPL (CD signal positive relating to R/D configuration) or to a lesser extent (CD signal negative relating to S/L configuration). The KM was used for the CD measurement. The concentration of KM was 7 mg/ml in water. The starting material (R)-3-hydroxybutyric acid, also known as D-β-hydroxybutyric acid was chiral and introduced chirality in KM. Circular Dichroism (CD) spectrum of KM was positive indicating R/D configuration in KM (FIG. 13).
KM assessment: Analysis of KM showed 80-70% of the monoglyceride based on NMR and GC-MS respectively. There were no diglycerides and triglycerides based on GC-MS analysis. 10-20% glycerol and 10% 3-hydroxybutyric acid starting materials were also seen in NMR and GC-MS respectively. No other impurities were seen.
Quality Control Parameters: Based on the research on quality control parameters for the Ketone Molecule (KM), GC-MS and LC-MS analysis were important quality control and characterization tools in the full characterization of green nanotechnology-based KM encapsulations within plant proteins.

### Example 4a—NR Characterization

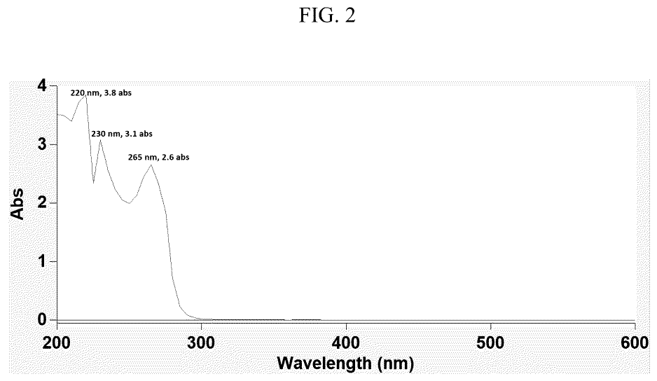

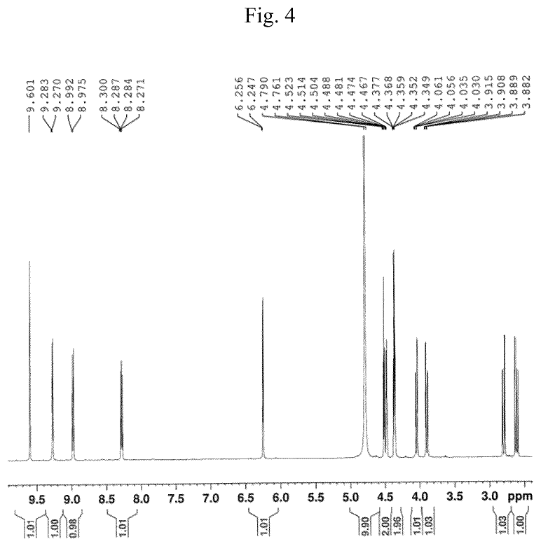

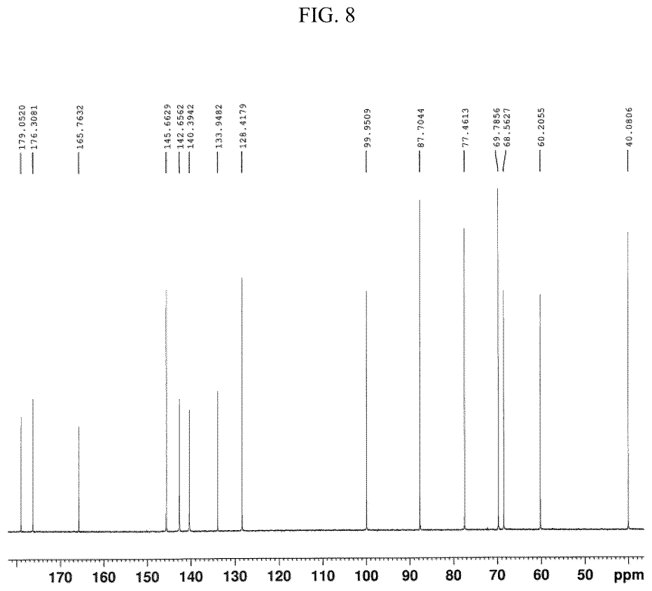

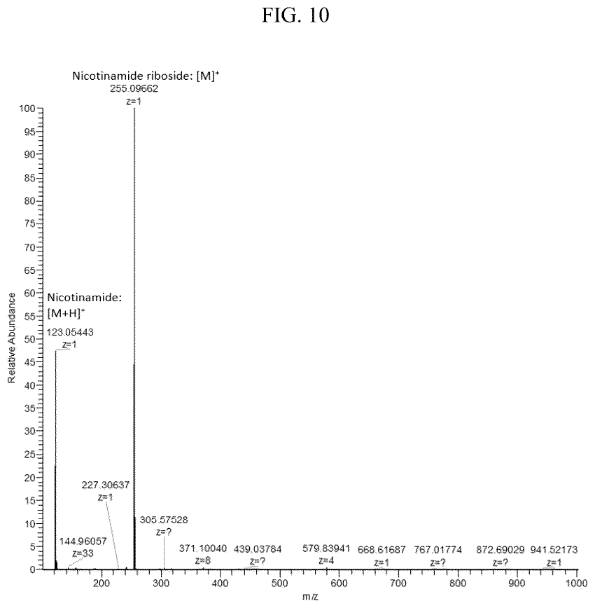

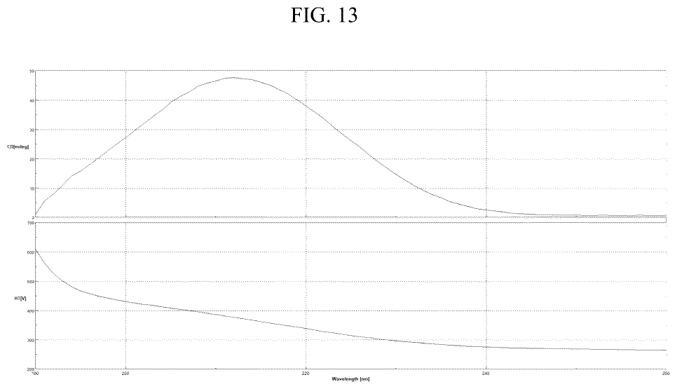

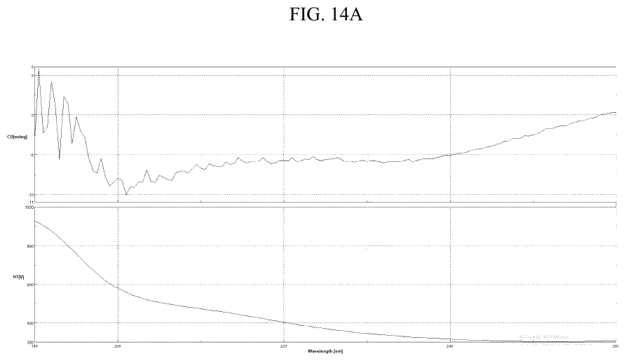

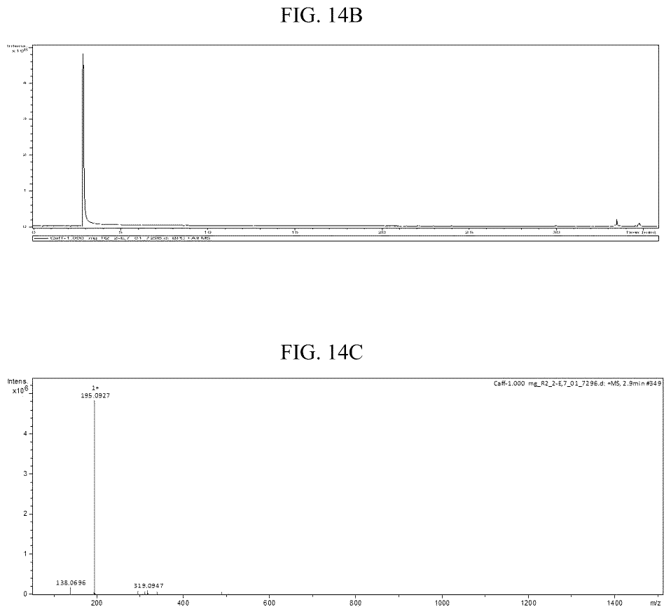

Appearance: NR was a fine white powder.
Smell: NR's smell was neutral.
Solubility: NR was water soluble and is miscible with ethanol. The chloride salt of nicotinamide riboside has good solubility in water.
pH: NR displayed a pH of 4.0 in water indicating an acidic aqueous solution.
UV-visible absorption spectroscopy: The electronic transition in NR pertains to the ultraviolet region (UV) based on the UV-visible absorption measurement. NR showed a broad absorption until ˜300 nm in the UV region with peaks at 220 nm, 230 nm, and 265 nm (FIG. 2). There was no chromophoric characteristic absorption peaks in the visible region. Therefore, with regards to tracking NR encapsulated protein nanoparticles, UV-visible spectroscopy was not utilized.
Nuclear magnetic resonance spectroscopy: ¹H nuclear magnetic resonance (NMR) spectra was recorded at 500 MHz on a Bruker 500 MHz spectrometer. ¹³C NMR spectra was recorded at 126 MHz on a Bruker 500 MHz spectrometer. NMR spectra was recorded in deuterated water (D₂₀). The 1H chemical shifts were reported relative to internal D₂O (FIG. 4). The ¹³C NMR chemical shifts were reported relative to an external tetramethylsilane (TMS) standard (FIG. 8). NR's proton and carbon NMR both showed a clean spectrum with no impurities.
Mass Spectrometry: A range of sophisticated mass spectrometric techniques had been utilized to develop a reliable quality control parameters for NR and its protein encapsulated products. Analysis of NR using electrospray ionization-mass spectrometry (ESI-MS) technique had been carried out. Apart from obtaining the elemental/isotopic signature of NR using MS, the [M+H]+ peak had been used for molecular weight estimation. The nicotinamide riboside anion [M]⁺ peak of 255.09 was detected as the major peak (FIG. 10). The peak at 123.05 correlated to the nicotinamide [M+H]⁺ fragment peak in NR.
Circular Dichroism (CD): NR was assessed by circular dichroism. The concentration of NR was 0.2 mg/ml in water. The circular Dichroism spectrum of NR was negative indicating S/L configuration (FIG. 14). The anionic portion of NR might also introduce chirality.
NR assessment: Analysis of NR showed nicotinamide riboside containing compound. NMR showed a clean spectrum with no starting materials or solvent impurities. Mass spectrometric analysis showed peaks correlating to nicotinamide riboside and nicotinamide units. Circular Dichroism (CD) spectrum of NR was negative indicating S/L configuration.
Quality Control Parameters: Based on the research on quality control parameters for the Nicotinamide Riboside (NR), the above analytical tools were important for various NR-related product development through green nanotechnology-based NR encapsulations.

### Example 4b—CA Characterization

Appearance: CA was a fine white powder.
Smell: CA's smell was neutral and taste is bitter.
Solubility: CA was water soluble (25 mg/ml).
pH: CA displayed a pH of 7.0 in water indicating an neutral aqueous solution
Liquid chromatography-mass spectrometry (LC-MS): Liquid chromatography-mass spectrometry (LC-MS) was utilized for the quality control confirmation of CA. LC-MS technique is an excellent analytical and quality control tool in detecting CA purity, amount and the presence of other materials. In the LC-MS analysis (FIG. 14B). The predominant peak at 3.0 min RT corresponded to the [M+H]⁺ peak of 195—corroborating results from literature for caffeine.
CA assessment: LC-MS analysis of CA showed pure material. CA is a well established compound and other elaborate characterization data can be obtained from National Institute of Standards and Technology (NIST) and Spectral Database for Organic Compounds (SDBS) websites.
Quality Control Parameters: Based on the research on quality control parameters for the Caffeine (CA), the LC-MS analytical tool was important for various CA-related product development through green nanotechnology-based CA encapsulations.

### Example 4b—TH Characterization

Appearance: TH was a fine white powder.
Smell: TH's smell was neutral and taste extremely bitter.
Solubility: CA was water soluble (25 mg/ml).
pH: CA displayed a pH of 7.0 in water indicating an neutral aqueous solution
Liquid chromatography-mass spectrometry (LC-MS): Liquid chromatography-mass spectrometry (LC-MS) was utilized for the quality control confirmation of TH. LC-MS technique is an excellent analytical and quality control tool in detecting TH purity, amount and the presence of other materials. In the LC-MS analysis (FIG. 14C). The predominant peak at 3.0 min RT corresponded to the [M+H]⁺ peak of 225—corroborating results from literature for theacrine.
TH assessment: LC-MS analysis of TH showed pure material. TH is a well established compound and other elaborate characterization data can be obtained from National Institute of Standards and Technology (NIST) website.
Quality Control Parameters: Based on the research on quality control parameters for the Theacrine (TH), the LC-MS analytical tool was important for various TH-related product development through green nanotechnology-based TH encapsulations.

### Example 5—Plant-Derived Protein Characterization

TABLE 2
Sources of plant-derived protein

| | |
|---|---|
| Water Soluble Pea | Water Soluble Protein— |
| Protein (PP) Powder | www.watersolubleprotein.com/ |
| | water-soluble-pea-protein-powder/ |
| Soy Protein Isolate | MP Biomedicals soy protein isolated |
| | www.fishersci.com/shop/products/mp- |
| | biomedicals-soy-protein-isolated-3/p-3533233 |
| | ICN90545605 |
| Okra Extract Powder | Bulk Supplements |
| | www.bulksupplements.com/products/okra- |
| | extract?variant=32133367070831 |

### Pea Protein

Water Soluble Pea Protein (PP) was purchased from WaterSolubleProtein.com website. The botanical source was Pisum sativum, natural and non-GMO yellow pea, which had been hydrolyzed to be highly water soluble (100 mg/ml). The protein solution was a clear yellow colored liquid with no sediments or residue. It had 80% protein content. It was edible and instantly water soluble and forms stable solutions.

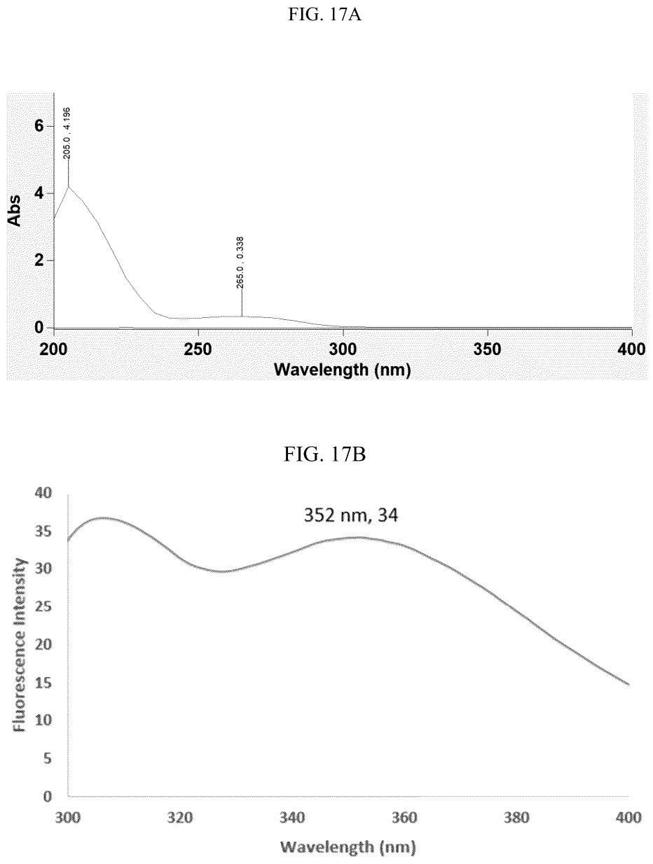

Pea Protein UV-visible absorption spectroscopy: The electronic transitions in water soluble Pea Protein (PP) pertains to the ultraviolet region (UV) based on the UV-visible absorption measurement. PP showed a weak absorption peak at 265 nm and a broad absorption in the 250 nm-200 nm UV region (FIG. 17A). There was no characteristic absorption peaks in the visible region.

Pea Protein fluorescence spectroscopy: Phenylalanine, tyrosine, and tryptophan amino acids in proteins gave rise to intrinsic protein fluorescence. The fluorescence emission spectra of water-soluble Pea Protein (PP) was recorded by excitation at 285 nm corresponding to the tryptophan amino acid. A broad emission peak at 352 nm was seen in the emission spectra of PP (FIG. 17B). Changes in the tryptophan microenvironment could be monitored through fluorescence spectroscopy. A shift in fluorescence maximum peak and variation in fluorescence intensity could give insight into pea protein interaction with ligands (Reference: Akbar, S. M. et al, Journal of Bioenergetics and Biomembranes, 2016, 48, 241-247 and Vivian, J. T. et al, Biophysical Journal 2001, 80, 2093-2109).

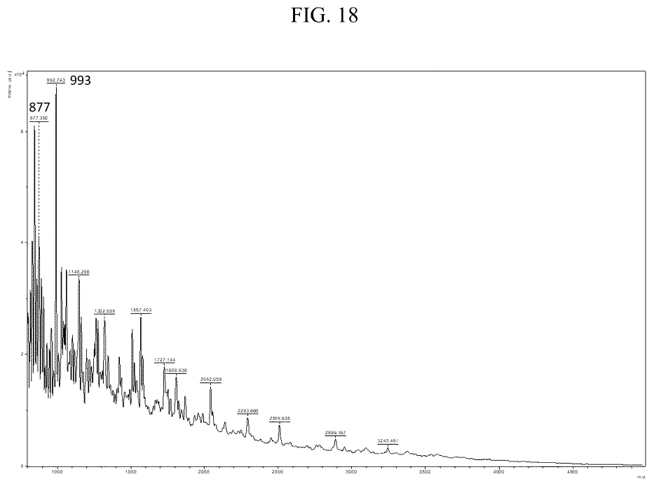

Pea Protein Matrix-Assisted Laser Desorption/Ionization-Mass Spectrometry (MALDI-MS): Water Soluble Pea Protein (PP) was analyzed using matrix-assisted laser desorption/ionization-mass spectrometry (MALDI-MS) to check the molecular weight of the protein components. Sinapic acid matrix was used. Mass range from 2 kDa-400 kDa was investigated on a Bruker AutoFlex Speed MALDI-MS and no significant peaks were seen. Then mass range was reduced to 5 kDa, and peak around 877 and 993 m/z was seen indicating protein molecular weight below ˜1 kDa (FIG. 18).

Pea Protein NMR. FIG. 41 showed a ¹H NMR of water-soluble Pea Protein (PP) in D2O.

### Soy Protein Isolate

Soy protein isolate was obtained from MP Biomedicals, Catalog No. ICN90545605. Soy protein is an abundant and cheap plant protein obtained from soybean. Soy consumption is beneficial for heart health and reduces inflammation. Hence for diabetes, atherosclerosis and cancer treatment soy have significant applications. Soy Protein Isolate (SPI) is a soy food product with the maximum soy protein content (92%). SPI is water soluble and is a useful drug delivery vehicle for water soluble pharmaceuticals. SPI is considered generally safe pharmaceuticals, biodegradable cheap and abundantly available too. Soy protein's anti-inflammatory properties can also be harnessed in the nano-therapeutic formulation.

Soy Protein Isolate UV-visible absorption spectroscopy: The electronic transitions SPI pertains to the ultraviolet region (UV) based on the UV-visible absorption measurement. SPI showed a broad absorption in the 240 nm-200 nm UV region (FIG. 42). There was no characteristic absorption peaks in the visible region.

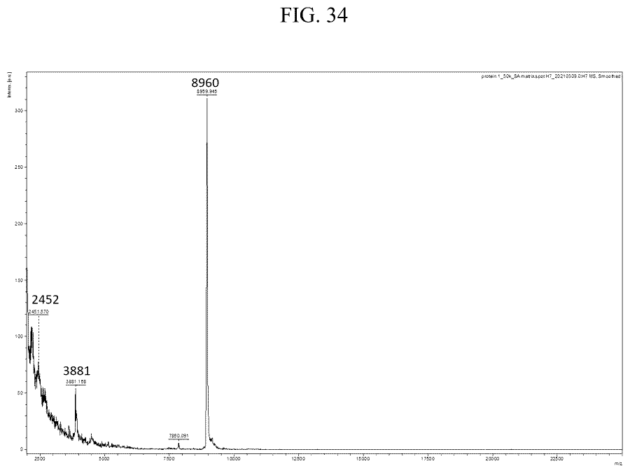

Soy Protein Isolate Matrix-Assisted Laser Desorption/Ionization-Mass Spectrometry (MALDI-MS): Water Soluble Pea Protein (PP) was analyzed using matrix-assisted laser desorption/ionization-mass spectrometry (MALDI-MS) to check the molecular weight of the protein components. Sinapic acid matrix was used. Mass range from 2 kDa-400 kDa was investigated on a Bruker AutoFlex Speed MALDI-MS. The significant peak was seen at 8960 m/z indicating protein molecular weight ˜9 kDa (FIG. 34).

### Okra Extract

Okra extract was obtained from Bulk Supplements, SKU OKR25 kg. Okra is a flowering plant belonging in the mallow family. Its green seed pods are edible and consists of water (90%), protein (2%), carbohydrates (7%) and trace amounts of fat. It is a rich source of dietary fiber, vitamin C and vitamin K. Okra mucilage or gel from the pods is a natural polysaccharide and includes D-galactose, L-rhamnose and L-galacturonic acid. Okra mucilage be extracted in water and forms a gelatinous viscous solution. Natural polysaccharides are biocompatible, non-toxic, and have biogenic polymeric applications in the form of microspheres, nano-matrix and nanoparticles. Okra Extract (OE), a finely ground dry powder was purchased from BulkSupplements.com website. It was edible, water soluble and can be stored at room temperature. OE generated a faintly cloudy brown solution of okra mucilage or gel in water. Natural polysaccharides like okra gel could be used for encapsulation, stabilization and drug delivery applications.

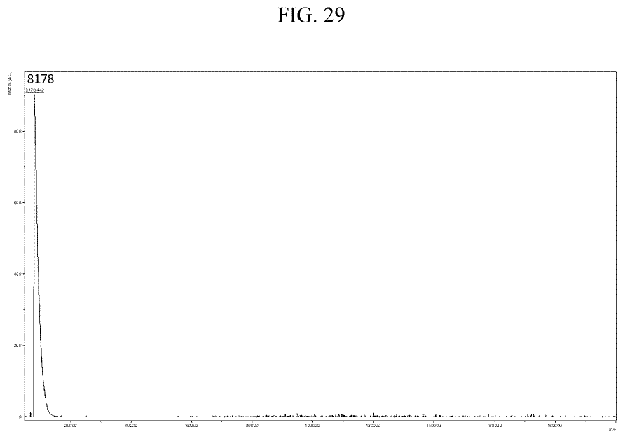

Okra mucilage Matrix-Assisted Laser Desorption/Ionization-Mass Spectrometry (MALDI-MS): Okra mucilage or gel from the green seed pods was extracted in water. It was a gelatinous viscous solution. Okra mucilage was analyzed using matrix-assisted laser desorption/ionization-mass spectrometry (MALDI-MS) to check the molecular weight of the protein components. Sinapic acid matrix was used. Mass range from 2 kDa-400 kDa was investigated on a Bruker AutoFlex Speed MALDI-MS. A peak around ˜8 kDa was seen for the okra polysaccharide mucilage (FIG. 29).

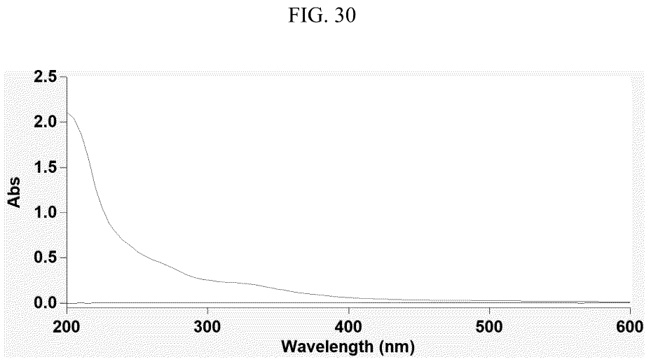

Okra mucilage UV-visible absorption spectroscopy: The electronic transitions in okra mucilage pertains mostly to the ultraviolet region (UV) based on the UV-visible absorption measurement. Okra Mucilage showed a weak broad absorption between 400 nm-300 nm. Further in the UV region it showed a more intense broad absorption in the 300 nm-200 region (FIG. 30). There was no characteristic absorption peaks in the visible region.

### Example 6—Phytochemical Characterization

Proton (1H) NMR spectra was recorded at 600 MHz on a Bruker 600 MHz spectrometer. Carbon (13C) NMR spectra was recorded at 151 MHz on a Bruker 600 MHz spectrometer. NMR spectra was recorded in deuterated water (D2O) or deuterated methanol (CD3OD) or a combination of both. The 1H chemical shifts were reported relative to internal D2O or CD3OD. The 13C NMR chemical shifts were reported relative to an external tetramethylsilane (TMS) standard. The carbon NMR showed corresponding changes.

Hydrogen bonding is a feature of KM, protein and phytochemical structures. Both intermolecular and intramolecular hydrogen bonding opportunities are extensive. Hydrogen bonding could be conducive for KM interaction with protein and phytochemical for nanoparticles synthesis and encapsulations purposes. Extensive hydrogen bonding feature facilitated KM nanoencapsulation and interaction of KM with protein and phytochemical (FIG. 30). It was also favorable for interaction of protein and phytochemical as well.

FIG. 42 showed a ¹H NMR of mangiferin in CD₃OD.

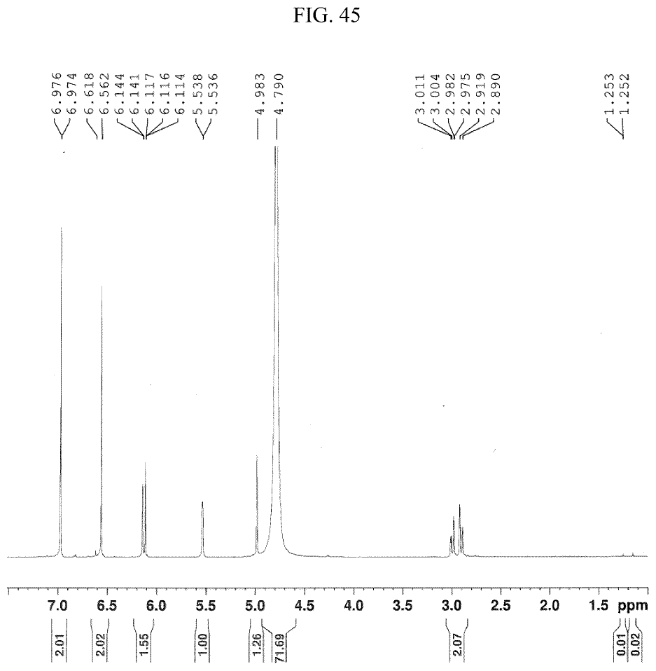

FIG. 45 showed a ¹H NMR of epigallocatechin gallate (EGCG) in D₂O.

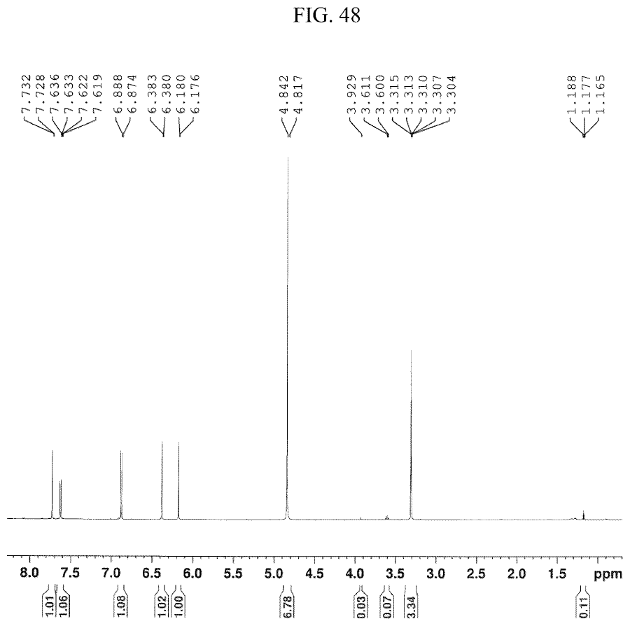

FIG. 48 showed a ¹H NMR of quercetin in CD₃OD.

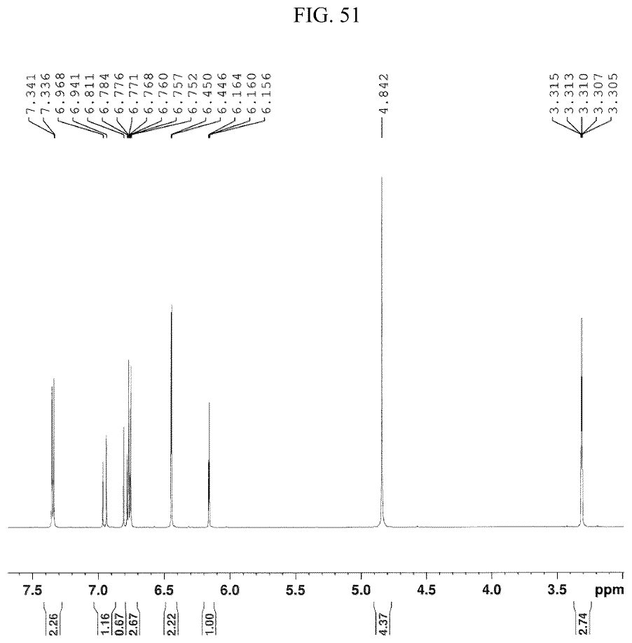

FIG. 51 showed a ¹H NMR of resveratrol in CD₃OD.

Phytochemicals were sourced from the following:

TABLE 3
Sources of Phytochemicals

| | |
|---|---|
| Loose-Leaf | Wagh Bakri Black Premium Loose Tea From |
| Tea | Assam Special International Blend (1 Lb) |
| | www.amazon.com/Wagh-Bakri-Premium-Special- |
| | International/dp/B002ANP672/ref=sr_1_37?crid=2P3EC7AUHoXV.J&dchild= |
| | 1&keywords=bulk%2Bassam%2Bblack%2Btea&qid=162962808&3sprefix= |
| | bulk%2Bassam%2Btea%2B%2Caps%2C164&sr=8-37&th=1 |
| | Darjeeling loose-leaf Tea, Bulk, Non-GMO (8 oz) |
| | https://www.amazon.com/Darjeeling-Tea-Organic-Loose- |
| | Servings/dp/B01FGPR1QE |
| Black Berry | Jungle Powders Blackberry Powder 3.5 oz, 100% Natural Whole Blackberry |
| Powder | Extract |
| | www.amazon.com/Jungle-Powders-Blackberry-Antioxidant- |
| | Concentrate/dp/B089QPQ5D8 |
| White | Grocery Store |
| Grapes | |
| Mango | Bulk Supplements |
| Powder | www.bulksupplements.com/products/mango- |
| | powder?_ pos=1&_sid=f4c41ebcd&_ss=r |
| | SKU MANP100 |
| Okra Extract | Bulk Supplements |
| Powder | www.bulksupplements.com/products/okra-extract?variant=32133367070831 |

### Example 7—Crosslinking of Pea Protein with Mangiferin and Encapsulation of Ketone Molecules in Pea Protein Crosslinked with Mangiferin

Synthesis of KM-encapsulated Pea Protein Nanoparticles by Mango Peel crosslinking (KM-PP-MP-NP): 15 g water soluble Pea Protein (PP), 15 g Mango Peel (MP) powder, and 6 g Ketone Molecule (KM) were weighed and transfer to a 2 L conical flask with a stir bar. 1 L distilled water was added to the flask and let the reaction mixture contents were stirred overnight. Mixing was stopped and the insoluble residue was then allowed to settle down. The insoluble residue was separated from the solution by decantation first and then filtration (75-micron size). 625 mL of the filtered solution was transferred to a 2 L round bottom flask with a stir bar. 210 mL of absolute ethanol (one third volume of filtered solution) was added to the flask contents and stirred vigorously. Then the reaction mixture were stirred overnight. The resulting KM encapsulated Pea Protein Nanoparticles by Mango Peel crosslinking (KM-PP-MP-NP) were then characterized by TEM, DLS size and ZP measurements. The KM content was estimated through GC-MS analysis and neat KMst.

Gas chromatography-mass spectrometry (GC-MS) procedure: 1 μL of KM sample to be analyzed was diluted 1000 times by adding 1000 μL of pyridine. 25 μL of this solution was trimethylsilylated with 25 μL MSTFA [N-methyl-N-(trimethyl-silyl)trifluoroacetamide]+ 1% TMCS (chlorotrimethylsilane) reagent by incubating at 50° C. for an hour. The derivatized samples were then analyzed using an Agilent 6890 GC coupled to a 5973N MSD mass spectrometer with a scan range from 50 to 650 m/z (Agilent Technologies, Inc., Santa Clara, CA). 1 μl of sample was injected into the GC column with a split ratio of 1:1 with a 60 min run time. Separation was achieved with a temperature program of 80° C. for 2 min, then ramped at 5° C./min to 315° C. and held at 315° C. for 12 min, on a 60 m DB-5 MS column (J&W Scientific, 0.25 mm ID, 0.25 um film thickness) and a constant flow of 1.0 ml/min of helium gas. A standard alkane mix was used for GC-MS quality control and retention index calculations. The chromatographic peaks in sample were deconvoluted using AMDIS and annotated through mass spectral and retention index matching to an in-house constructed spectral library and commercial NIST17 mass spectral library. Neat KM standard with five different concentrations prepared in pyridine were similarly analyzed. Peak area was calculated using Agilent, Enhanced Data Analysis (EDA) software to generate a calibration curve. Using the calibration curve and the peak area of the GC peak of the sample solution at RT˜30 min, the concentration of the monoglycerides in KM solutions were accurately calculated.

Standard calibration. The KM-PP-MP-NP solution was refrigerated (5° C.±3° C.) for extending shelf life. Blank Pea Protein Nanoparticles by Mango Peel crosslinking (PP-MP-NP) were synthesized following the same procedure without addition of KM

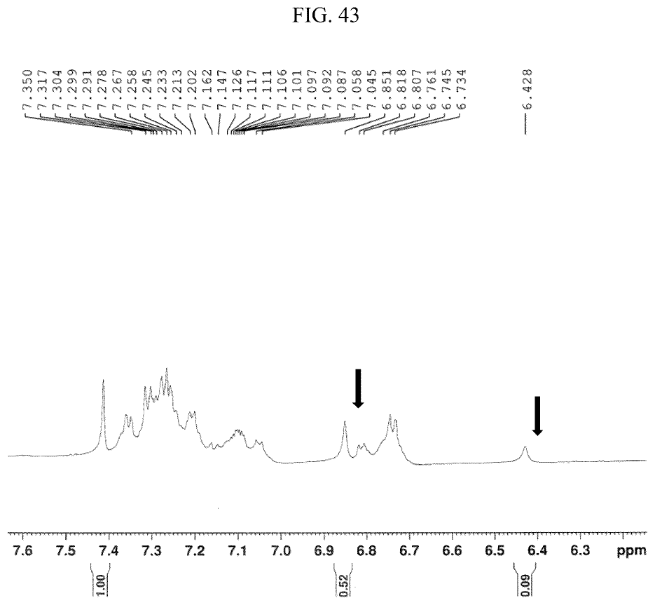

FIG. 43 showed a 1H NMR (expanded) of mangiferin and water-soluble Pea Protein (PP) reaction mixture in CD3OD/D2O (arrows indicate change in proton).

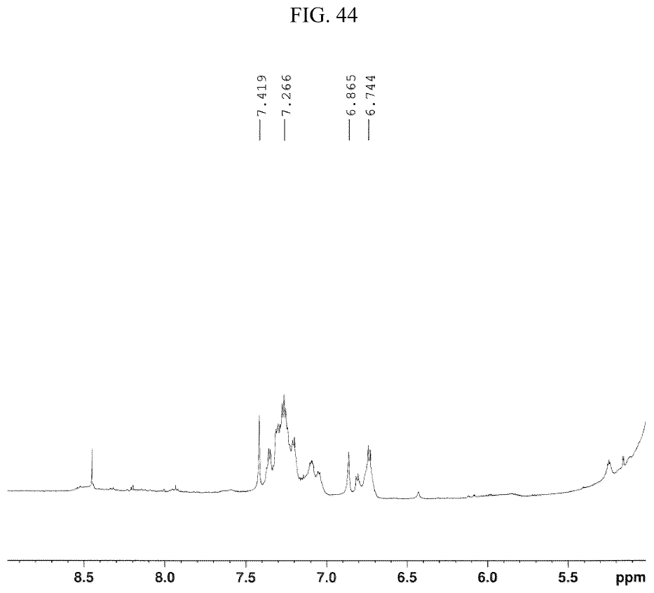

FIG. 44 showed a ¹H NMR (expanded) of mangiferin, water soluble Pea Protein (PP) and KM reaction mixture in CD₃OD/D₂O.

The aromatic ring protons of the phytochemical showed distinct changes indicating covalent interaction with the protein. These changes led to crosslinking with protein amino functional groups. The mangiferin proton NMR showed equivalent peaks at 7.5, 6.8 and 6.4 ppm. But after reaction with the pea protein there was significant change in the 6.8 and 6.4 ppm peaks.

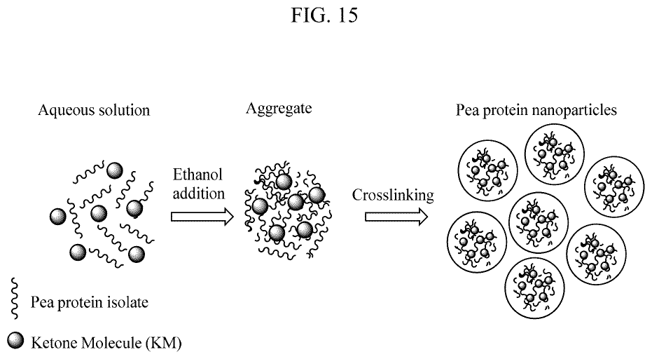

KM encapsulated Pea Protein Nanoparticles by Mango Peel crosslinking (KM-PP-MP-NP): Naturally available plant-based materials were explored for crosslinking. Mangiferin (MGF), a phytochemical found in mango, was extracted from the mango peel. The multiple hydroxyl groups in MGF were effective for protein crosslinking. KM encapsulated water-soluble Pea Protein Nanoparticles using Mango Peel crosslinking (KM-PP-MP-NP) was attempted by ethanol desolvation method (FIG. 15). After nanoparticle synthesis, any insoluble residue was removed by centrifugation/filtration. KM-PP-MP-NP size was between 336-422 nm, with an average of 367 nm (using DLS) and showed an average zeta potential of −15 mV (Table 4) indicating reasonable stability. Polydispersity index (PDI) of 0.2 indicates nanoparticles of uniform size (unimodal distribution). Using negative staining technique nanoparticle core size was explored by TEM (FIG. 23). It showed good particles with size in the ˜400 nm range. KM-PP-MP-NP was formulated and fully characterized to define quality control parameters for use in scale-up and commercial level productions.

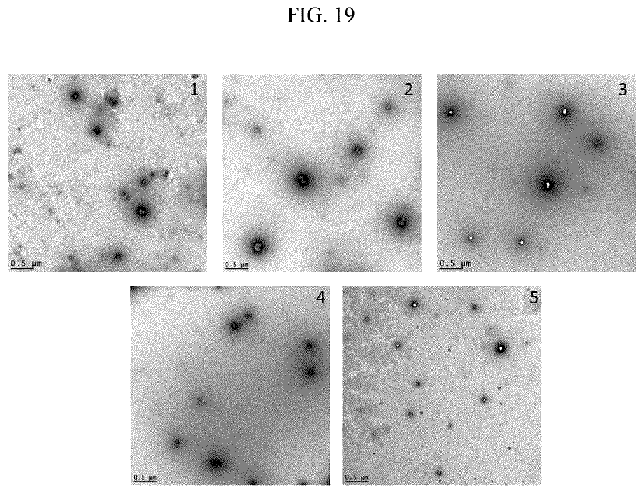

The particles showed an average size of 428 nm (using DLS) and an average zeta potential of −16 mV (Table 4), indicating reasonable stability. PDI of 0.2 indicated nanoparticles of uniform size (unimodal distribution). Using a negative staining technique, nanoparticle core size was explored by TEM (FIG. 19). It showed good particles with size in the ˜400 nm range. Blank nanoparticles were comparable to KM encapsulated nanoparticles with a slight increase in size. Blank Pea Protein Nanoparticles by Mango Peel crosslinking were synthesized with no KM for comparison (Serial No. 5).

TABLE 4
Characterization of KM encapsulated Pea Protein Nanoparticles by ethanol
desolvation method and Mango Peel crosslinking (KM-PP-MP-NP).

| | | | | | | | | DLS | | |
|---|---|---|---|---|---|---|---|---|---|---|
| Serial | PP | KM | MP | Water | Ethanol | PP:KM:MP | Water:Ethanol | Size | | ZP |
| No. | (mg/mL) | (mg/mL) | (mg/mL) | (mL) | (mL) | Ratio | Ratio | (nm) | PDI | (mV) |
| 1 | 15 | 5 | 17^{a} | 47 | 16 | 3:1:3 | 3:1 | 343 | 0.2 | −18 |
| 2 | 15 | 6 | 15^{a} | 625 | 210 | 3:1:3 | 3:1 | 336 | 0.2 | −17 |
| 3 | 15 | 0 | 17^{a} | 42 | 14 | 3:0:3 | 3:1 | 369 | 0.2 | −19 |
| 4 | 100 | 300 | 15 | 1000 | 350 | 0.3:1:0.1 | 3:1 | 422 | 0.2 | −9 |
| 5 | 100 | 0 | 17 | 5 | 2 | 6:0:1 | 3:1 | 486 | 0.3 | −12 |

^{a}= mango peel (MP) obtained from finely ground dried mango peel obtained from mango fruit

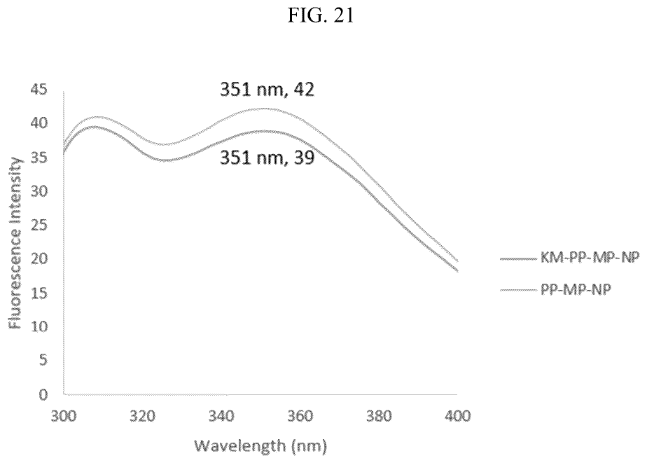

KM-PP-MP-NP and PP-MP-NP fluorescence spectroscopy: The fluorescence emission spectra of KM-PP-MP-NP and PP-MP-NP were recorded by excitation at 285 nm corresponding to the tryptophan amino acid. A broad emission peak at 351 nm with fluorescence intensity of 40 was seen in the emission spectra of (FIG. 21). Changes in the tryptophan microenvironment could be monitored through fluorescence spectroscopy. KM-PP-MP-NP and PP-MP-NP fluorescence maximum peak and intensity were comparable indicating not much change in the tryptophan region of the pea protein nanoparticles. Since KM was water soluble and hydrophilic, it did not interact with the hydrophobic tryptophan microenvironment of the pea protein. The fluorescence measurements gave insights into pea protein interaction with KM.

GC-MS analysis of KM encapsulated Pea Protein Nanoparticles by Mango Peel crosslinking (KM-PP-MP-NP): The amount of KM in the nanoparticle formulation was established using GC-MS analysis. It was found to be quantitative, indicating 100% KM encapsulation. A high KM encapsulation efficiency might be important for bio-delivery of KM.

Extensive hydrogen bonding is a feature of KM, protein and phytochemical structures. This is not surprising considering the ester, hydroxyl, amine and oxo functional groups in their structures. Both intermolecular and intramolecular hydrogen bonding opportunities are extensive. Hydrogen bonding is very conducive for KM interaction with protein and phytochemical for nanoparticles synthesis and encapsulations purposes. Extensive hydrogen bonding feature facilitated KM nanoencapsulation and interaction of KM with protein and phytochemical (FIG. 39). It was also favorable for interaction of protein and phytochemical as well.

### Example 8a—Crosslinking of Pea Protein with Mangiferin and Encapsulation of Nicotinamide Riboside Molecules in Pea Protein Crosslinked with Mangiferin

Synthesis of NR-encapsulated pea protein nanoparticles by mango peel crosslinking (NR-PP-MP-NP): 15 g of water-soluble pea protein (PP), 16 g mango peel (MP) powder, and 5 g nicotinamide riboside (NR) were weighed and transfer to a 2 L conical flask with a stir bar. 1 L distilled water was added to the flask, and the reaction mixture contents were stirred overnight. Mixing was stopped, and the insoluble residue was then allowed to settle. The insoluble residue was separated from the solution by decantation and then filtration (75-micron size). 700 mL of the filtered solution was transferred to a 2 L round bottom flask with a stir bar. 234 mL of absolute ethanol (one third volume of filtered solution) was added to the flask, and the contents were stirred vigorously. Then the reaction mixture were stirred overnight and any insoluble residue was removed by centrifugation/filtration (75-micron size). The resulting NR-encapsulated pea protein nanoparticles by mango peel crosslinking (NR-PP-MP-NP) were then characterized by TEM, DLS size and ZP measurements (Table 5). The NR-PP-MP-NP solution was refrigerated (5° C.±3° C.) for extending shelf life.

Standard calibration. Blank pea protein nanoparticles by mango peel crosslinking (PP-MP-NP) were synthesized following the same procedure without addition of NR.

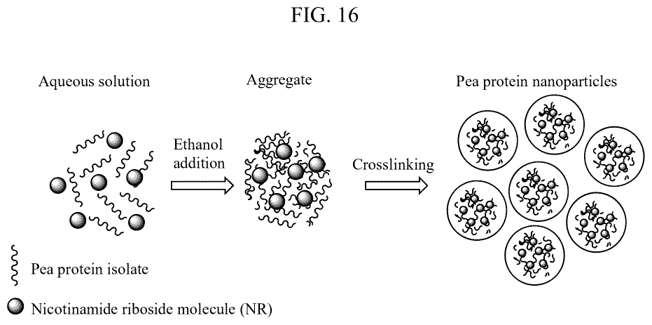

NR-encapsulated pea protein nanoparticles by mango peel crosslinking (NR-PP-MP-NP): Naturally available plant-based materials were explored for crosslinking. Mangiferin (MGF), a phytochemical found in mango, could be extracted from the mango peel. The multiple hydroxyl groups in MGF were effective for protein crosslinking. As noted above, NR-encapsulated water-soluble pea protein nanoparticles using mango peel crosslinking (NR-PP-MP-NP) was attempted by ethanol desolvation method (FIG. 16). After nanoparticle synthesis, any insoluble residue that was larger than 75 micron in size was removed by centrifugation/filtration. NR-PP-MP-NP size was between 286-369 nm, with an average of 297 nm (using DLS) and showed an average zeta potential of −18 mV (Table 5a) indicating reasonable stability. Polydispersity index (PDI) of 0.2 indicated nanoparticles of uniform size (unimodal distribution). Using negative staining technique, nanoparticle core size was explored by TEM (FIG. 20). It showed particles with size in the ˜300 nm range. NP-PP-MP-NP was formulated and fully characterized to define quality control parameters for use in scale-up and commercial level productions. Blank nanoparticles were comparable to NR encapsulated nanoparticles with a slight increase in size.

TABLE 5a
Characterization of NR encapsulated Pea Protein Nanoparticles by ethanol
desolvation method and Mango Peel crosslinking (NR-PP-MP-NP).

| | | | | | | | | DLS | | |
|---|---|---|---|---|---|---|---|---|---|---|
| Serial | PP | NR | MP | Water | Ethanol | PP:NR:MP | Water:Ethanol | Size | | ZP |
| No. | (mg/mL) | (mg/mL) | (mg/mL) | (mL) | (mL) | Ratio | Ratio | (nm) | PDI | (mV) |
| 1 | 15 | 5 | 15^{a} | 7 | 2 | 3:1:3 | 3:1 | 286 | 0.2 | −17 |
| 2 | 15 | 5 | 16^{a} | 700 | 234 | 3:1:3 | 3:1 | 308 | 0.2 | −18 |
| 3 | 15 | 0 | 17^{a} | 42 | 14 | 3:0:3 | 3:1 | 369 | 0.2 | −19 |

^{a}= mango peel (MP) obtained from finely ground dried mango peel

### Example 8b—Crosslinking of Pea Protein with Mangiferin and Encapsulation of Caffeine Molecules in Pea Protein Crosslinked with Mangiferin

Synthesis of CA-encapsulated pea protein nanoparticles by mango peel crosslinking (CA-PP-MP-NP): 25 g of water-soluble pea protein (PP), 8 g mango peel (MP) powder, and 13 g caffeine (CA) were weighed and transferred to a 1 L conical flask with a stir bar. 500 mL distilled water was added to the flask, and the reaction mixture contents were stirred overnight. Then the reaction mixture was stirred overnight, and any insoluble residue was removed by centrifugation/filtration (75-micron size). The resulting CA-encapsulated pea protein nanoparticles by mango peel crosslinking (CA-PP-MP-NP) were then characterized by TEM, DLS size and ZP measurements (Table 5b). The CA-PP-MP-NP solution was refrigerated (5° C.±3° C.) for extending shelf life.

CA-encapsulated pea protein nanoparticles by mango peel crosslinking (CA-PP-MP-NP): Naturally available plant-based materials were explored for crosslinking. Mangiferin (MGF), a phytochemical found in mango, could be extracted from the mango peel. The multiple hydroxyl groups in MGF were effective for protein crosslinking. CA-PP-MP-NP had an average size of 652 nm (using DLS) and showed an average zeta potential of −16 mV (Table 5b) indicating reasonable stability. Polydispersity index (PDI) of ˜0.3 indicated nanoparticles of mostly uniform size (unimodal distribution). Using negative staining technique, nanoparticle core size was explored by TEM (FIG. 20B). It showed longitudinal particles with size in the ˜200 nm range. CA-PP-MP-NP was formulated and fully characterized to define quality control parameters for use in scale-up and commercial level productions.

TABLE 5b
Characterization of CA encapsulated Pea Protein Nanoparticles
by Mango Peel crosslinking (CA-PP-MP-NP).

| | | | | | | DLS | | |
|---|---|---|---|---|---|---|---|---|
| Serial | PP | CA | MP | Water | PP:CA | Size | | ZP |
| No. | (mg/mL) | (mg/mL) | (mg/mL) | (mL) | Ratio | (nm) | PDI | (mV) |
| 1 | 50 | 25 | 15^{a} | 10 | 2:1 | 560 | 0.2 | −18 |
| 2 | 50 | 25 | 15^{a} | 500 | 2:1 | 743 | 0.5 | −13 |

^{a}= mango peel (MP) obtained from finely ground dried mango peel

### Example 8c—Crosslinking of Pea Protein with Mangiferin and Encapsulation of Theacrine Molecules in Pea Protein Crosslinked with Theacrine

Synthesis of TH-encapsulated pea protein nanoparticles by mango peel crosslinking (TH-PP-MP-NP): 25 g of water-soluble pea protein (PP), 8 g mango peel (MP) powder, and 13 g theacrine (TH) were weighed and transferred to a 1 L conical flask with a stir bar. 500 mL distilled water was added to the flask, and the reaction mixture contents were stirred overnight. Then the reaction mixture was stirred overnight, and any insoluble residue was removed by centrifugation/filtration (75-micron size). The resulting TH-encapsulated pea protein nanoparticles by mango peel crosslinking (TH-PP-MP-NP) were then characterized by TEM, DLS size and ZP measurements (Table 5c). The TH-PP-MP-NP solution was refrigerated (5° C.±3° C.) for extending shelf life.

TH-encapsulated pea protein nanoparticles by mango peel crosslinking (TH-PP-MP-NP): Naturally available plant-based materials were explored for crosslinking. Mangiferin (MGF), a phytochemical found in mango, could be extracted from the mango peel. The multiple hydroxyl groups in MGF were effective for protein crosslinking. TH-PP-MP-NP had an average size of 563 nm (using DLS) and showed an average zeta potential of −17 mV (Table 5c) indicating reasonable stability. Polydispersity index (PDI) of ˜0.5 indicated nanoparticles of slightly broad size distribution. Using negative staining technique, nanoparticle core size was explored by TEM (FIG. 20C). It showed particles with size in the ˜100 nm range. TH-PP-MP-NP was formulated and fully characterized to define quality control parameters for use in scale-up and commercial level productions.

TABLE 5c
Characterization of TH encapsulated Pea Protein Nanoparticles
by Mango Peel crosslinking (TH-PP-MP-NP).

| | | | | | | DLS | | |
|---|---|---|---|---|---|---|---|---|
| Serial | PP | TH | MP | Water | PP:TH | Size | | ZP |
| No. | (mg/mL) | (mg/mL) | (mg/mL) | (mL) | Ratio | (nm) | PDI | (mV) |
| 1 | 50 | 25 | 15^{a} | 10 | 2:1 | 553 | 0.6 | −20 |
| 2 | 50 | 25 | 15^{a} | 500 | 2:1 | 573 | 0.5 | −13 |

^{a}= mango peel (MP) obtained from finely ground dried mango peel

### Example 9—Crosslinking of Pea Protein with EGCG and Encapsulation of Ketone Molecules in Pea Protein Crosslinked with EGCG

KM encapsulated Pea Protein Nanoparticles were synthesized by Tea Extract crosslinking (KM-PP-TE-NP): Tea Extract (TE) solution was prepared for the nanoparticle synthesis. 6 g of Darjeeling loose-leaf tea (Table 3) was weighed and transferred to a 1 L conical flask. 300 ml distilled water was added to the flask, and the contents were boiled for 10 minutes at 100° C. The heating was then switched off and the tea extract solution was allowed to cool down to room temperature. The insoluble tea leaves residue from the solution were separated using Whatman paper filtration. The filtered, clear, deep-brown TE solution was utilized for the nanoparticle synthesis. 8 g water soluble Pea Protein (PP) was weighed and transferred to a 2 L conical flask with a stir bar. 500 mL distilled water was added to the flask and the pea protein solution was stirred for 30 minutes. Then 3 g Ketone Molecule (KM) was weighed and transferred to the flask. The solution was stirred for 30 more minutes. 100 mL of the TE solution was added, and the reaction mixture was stirred for another 2 hours. Then, 170 mL of absolute ethanol (one third volume of 500 ml water) was added to the flask contents whilst vigorous stirring. The reaction mixture was stirred overnight. The resulting KM-encapsulated Pea Protein Nanoparticles by Tea Extract crosslinking (KM-PP-TE-NP) was then characterized by TEM, DLS size and ZP measurements. The KM content was estimated through GC-MS analysis and neat KM standard calibration described above. The KM-PP-TE-NP solution was refrigerated (5° C.±3° C.) to extend the shelf life. Blank Pea Protein Nanoparticles by Tea Extract crosslinking (PP-TE-NP) were synthesized following the same procedure without addition of KM.

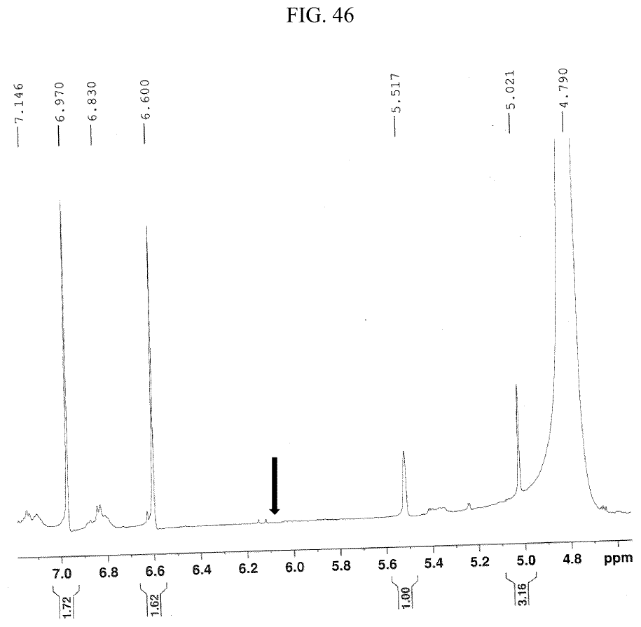

FIG. 46 showed a ¹H NMR (expanded) of epigallocatechin gallate (EGCG) and water-soluble Pea Protein (PP) reaction mixture in D₂O (arrows indicate change in proton).

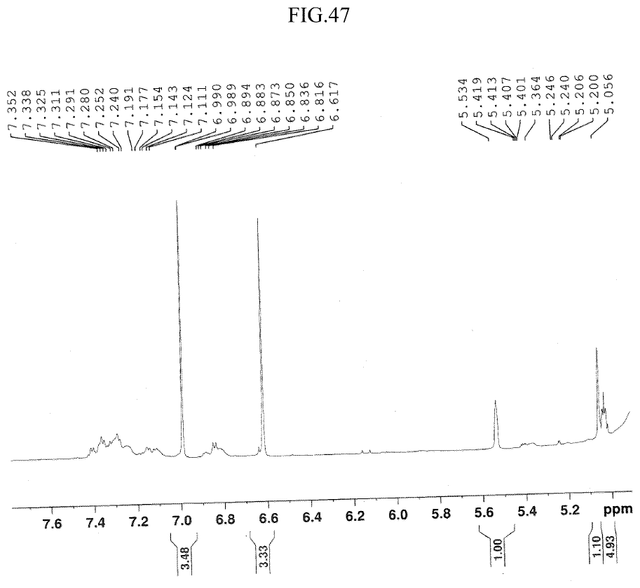

FIG. 47 showed a ¹H NMR (expanded) of epigallocatechin gallate (EGCG), water soluble Pea Protein (PP) and KM reaction mixture in D₂O.

The aromatic ring protons of the phytochemical showed distinct changes indicating covalent interaction with the protein. These changes led to crosslinking with protein amino functional groups. In the EGCG proton NMR the doublet peak at 6.1 ppm disappeared after reacting with the pea protein.

Tea leaves extract contain a variety of polyphenols including flavonoids, epigallocatechin gallate (EGCG) and other catechins. Hence, tea extract can be effective in crosslinking protein nanoparticles. KM-encapsulated water-soluble Pea Protein Nanoparticles using Tea Extract Crosslinking (KM-PP-TE-NP) was carried out by ethanol desolvation method. After nanoparticle synthesis, any insoluble residue was removed by centrifugation/filtration. Ethanol was removed from the formulation by rotatory evaporation at 37 degrees. KM-PP-TE-NP size was between 316-373 nm, with an average size of 338 nm (using DLS) and showed an average zeta potential of −13 mV (Table 6), indicating stability. Polydispersity index (PDI) of ˜0.2 indicates nanoparticles of uniform size (unimodal distribution). Using negative staining techniques, nanoparticle core size was estimated by TEM (FIG. 23). It showed stable particles with size in the ˜300 nm range. KM-PP-TE-NP was formulated and characterized to define quality control parameters for use in scale-up and commercial level productions. Initially small scale (˜10 mL and 50 mL) nanoparticles were formulated. After establishing characterization and quality control parameters, it was scaled up to ˜1 L. KM from repeated batches were utilized for the large-scale synthesis. Large scale batches showed reliable scale-up and characterization parameters.

GC-MS analysis of KM encapsulated Pea Protein Nanoparticles by Tea Extract crosslinking (KM-PP-TE-NP): The amount of KM in the nanoparticle formulation was established using GC-MS analysis. It was found to be quantitative indicating 100% KM encapsulation. A high KM encapsulation efficiency may be important for bio-delivery of KM. The GC-MS column provided good resolution of the starting materials and various glycerides. Quantification of KM could be carried out by using pure and neat KM to obtain a calibration curve. The curve had good linearity and the analysis concentration fell within the calibration range. GC-MS can be used for quality control of the KM encapsulation process.

Blank Protein Nanoparticles by Tea Extract crosslinking (PP-TE-NP): Blank Pea Protein Nanoparticles by Tea Extract crosslinking was synthesized with no KM for comparison. It showed a size of 362 nm (using DLS) and a zeta potential of −14 mV (Table 6) indicating reasonable stability. PDI of 0.1 indicates nanoparticles of uniform size (unimodal distribution). Using negative staining technique, nanoparticle core size was explored by TEM (FIG. 23). It showed good particles with size in the ˜400 nm range. Blank nanoparticles were comparable to KM encapsulated nanoparticles with a slight increase in size and potential.

TABLE 6
Characterization of KM encapsulated Pea Protein Nanoparticles by ethanol
desolvation method and Tea Extract crosslinking (KM-PP-TE-NP)

| | | | Tea | | | | | | | |
|---|---|---|---|---|---|---|---|---|---|---|
| | | | Extract | | | | | DLS | | |
| Serial | PP | KM | (TE) | Water | Ethanol | PP:KM | Water:Ethanol | Size | | ZP |
| No. | (mg/mL) | (mg/mL) | (mL) | (mL) | (mL) | Ratio | Ratio | (nm) | PDI | (mV) |
| 1 | 15 | 5 | 2^{a} | 10 | 3 | 3:1 | 3:1 | 316 | 0.3 | −17 |
| 2 | 15 | 5 | 10^{a} | 50 | 17 | 3:1 | 3:1 | 320 | 0.2 | −15 |
| 3 | 15 | 5 | 100^{a} | 500 | 170 | 3:1 | 3:1 | 342 | 0.2 | −14 |
| 4 | 15 | 0 | 5^{a} | 25 | 9 | 3:0 | 3:1 | 362 | 0.1 | −14 |
| 5 | 50 | 300 | 2000 | 8000 | 2000 | 1:6 | 4:1 | 373 | 0.2 | −6 |

^{a}= Tea leaves (20 mg/mL) boiled at 100 degrees for tea extract (TE)

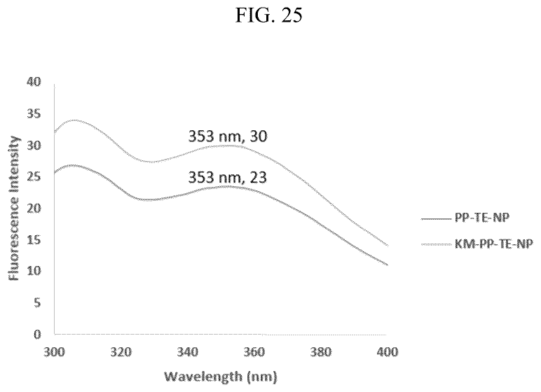

KM-PP-TE-NP and PP-TE-NP fluorescence spectroscopy: The fluorescence emission spectra of KM-PP-TE-NP and PP-TE-NP were recorded by excitation at 285 nm corresponding to the tryptophan amino acid. A broad emission peak at 353 nm with fluorescence intensity of 30 was seen in the emission spectra of (FIG. 25). Changes in the tryptophan microenvironment could be monitored through fluorescence spectroscopy. KM-PP-TE-NP and PP-TE-NP fluorescence maximum peak and intensity were comparable indicating not much change in the tryptophan region of the pea protein nanoparticles. Since KM was water soluble and hydrophilic, it did not interact with the hydrophobic tryptophan microenvironment of the pea protein. The fluorescence measurements gave insights into pea protein interaction with KM.

### Example 10—Crosslinking of Pea Protein with EGCG and Encapsulation of NR Molecules in Pea Protein Crosslinked with EGCG

NR-encapsulated pea protein nanoparticles were synthesized by tea extract crosslinking (NR-PP-TE-NP): Tea Extract (TE) solution was prepared for the nanoparticle synthesis by weighing 5 g of Darjeeling loose-leaf tea and transferring it to a 1 L conical flask. 250 ml distilled water was added to the flask, and the contents were boiled for 10 minutes at 100° C. The heating was switched off, and the tea extract solution was allowed to cool down to room temperature. The insoluble tea leaves residue from the solution were separated using Whatman paper filtration. The filtered, clear, deep-brown TE solution was utilized for the nanoparticle synthesis. 8 g water soluble Pea Protein (PP) was weighed and transferred to a 2 L conical flask with a stir bar. 500 mL distilled water was added to the flask, and the pea protein solution was stirred for 30 minutes. 3 g NR was weighed and transferred to the flask. The solution was stirred for 30 more minutes. Further, 100 mL of the TE solution was added, and the mixture was stirred for another 2 hours. Then, 170 mL of absolute ethanol (one third volume of 500 ml water) was added to the flask contents whilst vigorous stirring. Then the reaction mixture was stirred overnight. The resulting NR-encapsulated Pea Protein Nanoparticles by Tea Extract crosslinking (NR-PP-TE-NP) was then characterized by TEM, DLS size, and ZP measurements. The NR content was estimated through GC-MS analysis and neat KM standard calibration described above. The NR-PP-TE-NP solution was refrigerated (5° C.±3° C.) to extend the shelf life. Blank Pea Protein Nanoparticles by Tea Extract crosslinking (PP-TE-NP) were synthesized following the same procedure without addition of NR.

Tea leaves extract contain a variety of polyphenols like flavonoids, epigallocatechin gallate (EGCG), and other catechins. Hence, tea extract can be effective in crosslinking protein nanoparticles. NR-encapsulated water-soluble pea protein nanoparticles using tea extract crosslinking (NR-PP-TE-NP) was carried out by ethanol desolvation method. After nanoparticle synthesis, any insoluble residue that was larger than 75-micron in size was removed by centrifugation/filtration. NR-PP-TE-NP size was between 370-429 nm, with an average size of 415 nm (using DLS) and showed an average zeta potential of −14 mV (Table 7), indicating stability. Polydispersity index (PDI) of ˜0.2 indicated nanoparticles of uniform size (unimodal distribution). Using negative staining techniques, nanoparticle core size was estimated by TEM (FIG. 24). It showed stable particles with size in the ˜400 nm range. NR-PP-TE-NP was formulated and characterized to define quality control parameters for use in scale-up and commercial level productions. Initially small scale (˜10 mL) nanoparticles were formulated. After establishing characterization and quality control parameters, it was scaled up to ˜1 L. NR from repeated batches were utilized for the large-scale synthesis. Large scale batches showed reliable scale-up and characterization parameters.

Blank Protein Nanoparticles by Tea Extract crosslinking (PP-TE-NP): Blank Pea Protein Nanoparticles by Tea Extract crosslinking was synthesized with no NR for comparison. It showed a size of 370 nm (using DLS) and a zeta potential of −19 mV (Table 7a) indicating reasonable stability. PDI of 0.1 indicates nanoparticles of uniform size (unimodal distribution). Using negative staining technique, nanoparticle core size was explored by TEM (FIG. 16). It showed good particles with size in the ˜300 nm range. Blank nanoparticles were comparable to NR encapsulated nanoparticles with a slight increase in size and potential.

TABLE 7a
Characterization of NR-encapsulated pea protein nanoparticles by ethanol
desolvation method and tea extract crosslinking (NR-PP-TE-NP)

| | | | Tea | | | | | | | |
|---|---|---|---|---|---|---|---|---|---|---|
| | | | Extract | | | | | DLS | | |
| Serial | PP | NR | (TE) | Water | Ethanol | PP:NR | Water:Ethanol | Size | | ZP |
| No. | (mg/mL) | (mg/mL) | (mL) | (mL) | (mL) | Ratio | Ratio | (nm) | PDI | (mV) |
| 4 | 15 | 5 | 2^{a} | 10 | 3 | 3:1 | 3:1 | 400 | 0.2 | −15 |
| 5 | 15 | 6 | 100^{a} | 500 | 170 | 3:1 | 3:1 | 429 | 0.2 | −12 |
| 6 | 15 | 0 | 10^{a} | 50 | 17 | 3:0 | 3:1 | 370 | 0.2 | −19 |

^{a}= Tea leaves (20 mg/mL) boiled at 100 degrees for tea extract (TE)

### Example 11—Crosslinking of Pea Protein with EGCG and Encapsulation of Caffeine Molecules in Pea Protein Crosslinked with EGCG

CA-encapsulated pea protein nanoparticles were synthesized by tea extract crosslinking (CA-PP-TE-NP): Tea Extract (TE) solution was prepared for the nanoparticle synthesis by weighing 5 g of Darjeeling loose-leaf tea and transferring it to a 1 L conical flask. 250 mL distilled water was added to the flask, and the contents were boiled for 10 minutes at 100° C. The heating was switched off, and the tea extract solution was allowed to cool down to room temperature. The insoluble tea leaves residue from the solution were separated using Whatman paper filtration. The filtered, clear, deep-brown TE solution was utilized for the nanoparticle synthesis. 20 g water soluble Pea Protein (PP) was weighed and transferred to a 1 L conical flask with a stir bar. 400 ml distilled water was added to the flask, and the pea protein solution was stirred for 30 minutes. 10 g CA was weighed and transferred to the flask. The solution was stirred for 30 more minutes. Further, 100 mL of the TE solution was added, and the mixture stirred for overnight. The resulting CA-encapsulated Pea Protein Nanoparticles by Tea Extract crosslinking (CA-PP-TE-NP) was then characterized by TEM, DLS size, and ZP measurements. The CA-PP-TE-NP solution was refrigerated (5° C.±3° C.) to extend the shelf life.

Tea leaves extract contain a variety of polyphenols like flavonoids, epigallocatechin gallate (EGCG), and other catechins. Hence, tea extract can be effective in crosslinking protein nanoparticles. CA-encapsulated water-soluble pea protein nanoparticles using tea extract crosslinking (CA-PP-TE-NP) was carried out. After nanoparticle synthesis, any insoluble residue that was larger than 75-micron in size was removed by centrifugation/filtration. CA-PP-TE-NP had an average size of 330 nm (using DLS) and a zeta potential of −13 mV (Table 7b). Polydispersity index (PDI) of ˜0.4 indicated nanoparticles of fairly uniform size (unimodal distribution). Using negative staining techniques, nanoparticle core size was estimated by TEM (FIG. 24B). It showed longitudinal particles with size in the ˜500 nm range. CA-PP-TE-NP was formulated and characterized to define quality control parameters for use in scale-up and commercial level productions. Initially small scale (˜10 mL) nanoparticles were formulated. After establishing characterization and quality control parameters, it was scaled up to ˜500 mL. Large scale batches showed reliable scale-up and characterization parameters.

TABLE 7b
Characterization of CA-encapsulated pea protein nanoparticles
by tea extract crosslinking (CA-PP-TE-NP)

| | | | | | | DLS | | |
|---|---|---|---|---|---|---|---|---|
| Serial | PP | CA | TE | Water | PP:CA | Size | | ZP |
| No. | (mg/mL) | (mg/mL) | (mg/mL) | (mL) | Ratio | (nm) | PDI | (mV) |
| 1 | 50 | 25 | 10 | 40 | 2:1 | 346 | 0.5 | −13 |
| 2 | 50 | 25 | 100^{a} | 400 | 2:1 | 314 | 0.3 | −13 |

^{a}= Tea leaves (20 mg/mL) boiled at 100 degrees for tea extract (TE)

### Example 12—Crosslinking of Pea Protein with EGCG and Encapsulation of Theacrine Molecules in Pea Protein Crosslinked with EGCG

TH-encapsulated pea protein nanoparticles were synthesized by tea extract crosslinking (TH-PP-TE-NP): Tea Extract (TE) solution was prepared for the nanoparticle synthesis by weighing 5 g of Darjeeling loose-leaf tea and transferring it to a 1 L conical flask. 250 mL distilled water was added to the flask, and the contents were boiled for 10 minutes at 100° C. The heating was switched off, and the tea extract solution was allowed to cool down to room temperature. The insoluble tea leaves residue from the solution were separated using Whatman paper filtration. The filtered, clear, deep-brown TE solution was utilized for the nanoparticle synthesis. 20 g water soluble Pea Protein (PP) was weighed and transferred to a 1 L conical flask with a stir bar. 400 ml distilled water was added to the flask, and the pea protein solution was stirred for 30 minutes. 10 g TH was weighed and transferred to the flask. The solution was stirred for 30 more minutes. Further, 100 mL of the TE solution was added, and the mixture stirred for overnight. The resulting TH-encapsulated Pea Protein Nanoparticles by Tea Extract crosslinking (TH-PP-TE-NP) was then characterized by TEM, DLS size, and ZP measurements. The TH-PP-TE-NP solution was refrigerated (5° C.±3° C.) to extend the shelf life.

Tea leaves extract contain a variety of polyphenols like flavonoids, epigallocatechin gallate (EGCG), and other catechins. Hence, tea extract can be effective in crosslinking protein nanoparticles. TH-encapsulated water-soluble pea protein nanoparticles using tea extract crosslinking (TH-PP-TE-NP) was carried out. After nanoparticle synthesis, any insoluble residue that was larger than 75-micron in size was removed by centrifugation/filtration. TH-PP-TE-NP had an average size of 322 nm (using DLS) and a zeta potential of −12 mV (Table 7c). Polydispersity index (PDI) of ˜0.4 indicated nanoparticles of fairly uniform size (unimodal distribution). Using negative staining techniques, nanoparticle core size was estimated by TEM (FIG. 24C). It showed particles with size in the ˜100 nm range. TH-PP-TE-NP was formulated and characterized to define quality control parameters for use in scale-up and commercial level productions. Initially small scale (˜50 mL) nanoparticles were formulated. After establishing characterization and quality control parameters, it was scaled up to ˜500 mL. Large scale batches showed reliable scale-up and characterization parameters.

TABLE 7c
Characterization of TH-encapsulated pea protein nanoparticles
by tea extract crosslinking (TH-PP-TE-NP)

| | | | | | | DLS | | |
|---|---|---|---|---|---|---|---|---|
| Serial | PP | TH | TE | Water | PP:TH | Size | | ZP |
| No. | (mg/mL) | (mg/mL) | (mg/mL) | (mL) | Ratio | (nm) | PDI | (mV) |
| 1 | 50 | 25 | 10^{a} | 40 | 2:1 | 242 | 0.5 | −13 |
| 2 | 50 | 25 | 100^{a} | 400 | 2:1 | 401 | 0.2 | −10 |

^{a}= Tea leaves (20 mg/mL) boiled at 100 degrees for tea extract (TE)

### Example 13—Crosslinking of Pea Protein with Quercetin and Encapsulation of Ketone Molecules in Pea Protein Crosslinked with Quercetin

Synthesis of KM encapsulated Pea Protein Nanoparticles by Berry Extract crosslinking (KM-PP-BE-NP): an aqueous solution of Blackberry Extract (BE) for the nanoparticle synthesis was prepared. 100 g of dried purple berry powder was weighed and transferred to a 1 L conical flask. 1000 ml distilled water was added to the flask, and the contents were allowed boil for 15 minutes at 100° C. Then the heat was turned off, and the solution was allowed to cool down to room temperature, allowing any remaining insoluble berry fruit powder residues to settle down. The insoluble residues from the purple solution were separated by decantation and then filtration (use a strainer of 75-micron mesh or carry out centrifugation). For the nanoparticle synthesis, the filtered clear purple BE aqueous solution was used. 50.0 g water soluble Pea Protein (PP) and 300.0 g Ketone Molecule (KM) were weighed and transferred to a 2 L conical flask with a stir bar. 1000 mL of the BE extract aqueous solution were added to the flask, and the contents were boiled for 15 minutes at 100° C. Then, while stirring, 333 mL of absolute ethanol was added to the flask, and the reaction mixture contents were stirred overnight. The resulting KM encapsulated Pea Protein Nanoparticles by Berry Extract crosslinking (KM-PP-BE-NP) were characterized by TEM, DLS size and ZP measurements. The KM-PP-BE-NP solution was refrigerated (5±3° C.) for extending shelf life. Blank Pea Protein Nanoparticles by Berry Extract crosslinking (PP-BE-NP) was synthesized following the same procedure without addition of KM.

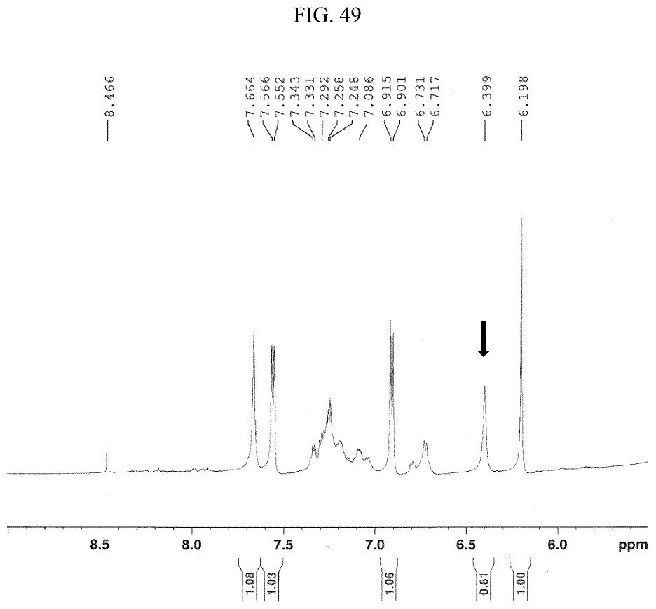

The quercetin proton NMR showed equivalent peaks at 7.7, 7.6, 6.9, 6.4 and 6.2 ppm. After reaction with the pea protein, the doublet peak at 6.4 ppm decreased significantly (FIGS. 48 and 49). NMR analysis clearly showed distinct change in quercetin indicating covalent interaction with the protein opening crosslinking pathways. The aromatic ring protons of the phytochemical showed distinct changes indicating covalent interaction with the protein. These changes led to crosslinking with protein amino functional groups. The quercetin proton NMR showed equivalent peaks at 7.7, 7.6, 6.9, 6.4 and 6.2 ppm. But after reaction with the pea protein, the doublet peak at 6.4 ppm decreased significantly.

The black berry extract containing the quercetin was a phytochemical cocktail containing various other active ingredients which can also play a role in the crosslinking and encapsulation process whilst adding nutraceutical value to the formulation.

FIG. 49 showed a ¹H NMR (expanded) of quercetin and water-soluble Pea Protein (PP) reaction mixture in CD₃OD/D₂O (arrows indicate change in proton).

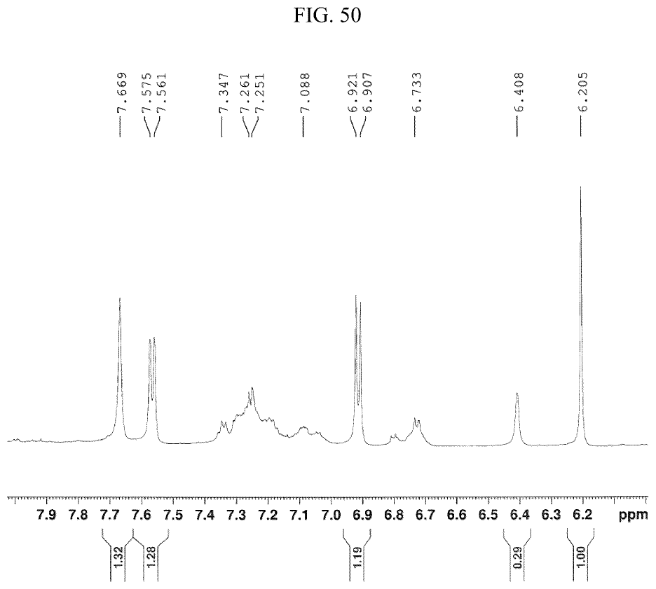

FIG. 50 showed a ¹H NMR (expanded) of quercetin, water soluble Pea Protein (PP) and KM reaction mixture in CD₃OD/D₂O.

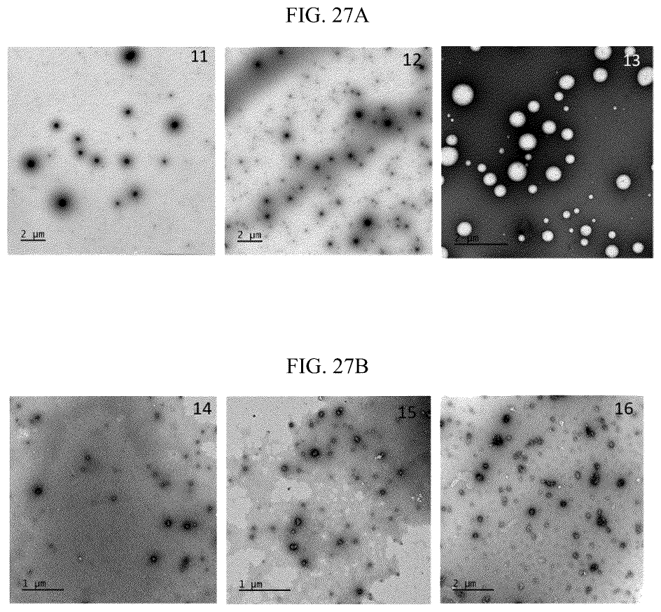

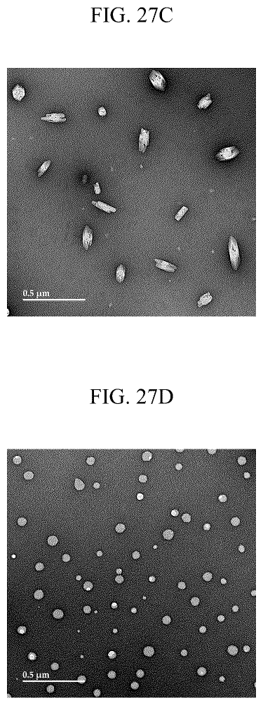

Crosslinking was investigated using naturally occurring plant-based materials from blackberries. Quercetin (QUE), a phytochemical present in berries could be extracted from berry extracts. The hydroxyl groups in QUE are effective at crosslinking proteins. The ethanol desolvation method was used to prepare KM encapsulated water-soluble Pea Protein Nanoparticles with Berry extract crosslinking (KM-PP-BE-NP). Centrifugation/filtration was used to eliminate any insoluble residues following nanoparticle production. KM-PP-BE-NP size between 392-474 nm with an average of was 433 nm (determined by DLS) and the average zeta potential of −9 mV (Table 8a), indicating reasonable stability. A polydispersity index (PDI) of 0.3 suggested mostly uniformly sized nanoparticles (unimodal distribution). TEM was used to investigate the size of nanoparticles using the negative staining technique nanoparticle (FIG. 27A). It showed a particle size in the range ˜100-500 nm. KM-PP-BE-NP was formulated and fully characterized to define quality control parameters for scale-up and commercial manufacturing. Initially, nanoparticles on a small scale (˜10 mL) were formulated. After establishing characterization and quality control parameters, it was successfully scaled up to ˜1 L to test large scale production. Large scale batches demonstrated reproducible scaling-up and characterization parameters. These experiments had aided in the definition of quality control parameters for usage at scale-up and commercial level productions.

TABLE 8a
Characterization of KM encapsulated Pea Protein Nanoparticles by ethanol

| | | | Berry | | | DLS | | |
|---|---|---|---|---|---|---|---|---|
| Serial | PP | KM | extract | Water | Ethanol | size | | ZF |
| No | (mg/mL) | (mg/mL) | (mL) | (mL) | (mL) | (nm) | PDI | (mV) |
| 11 | 50 | 300 | 10^{a} | 0 | 2 | 392 | 0.3 | −10 |
| 12 | 50 | 300 | 10^{b} | 0 | 2 | 474 | 0.3 | −9 |
| 13 | 50 | 0 | 10^{a} | 10 | 2 | 415 | 0.2 | −10 |

^{a}= Berry powdered extract (25 mg/ml ) boiled at too degrees
^{b}= Berry powdered extract (so mg/mL) boiled at soo degrees

### Example 14—Crosslinking of Pea Protein with Quercetin and Encapsulation of Caffeine Molecules in Pea Protein Crosslinked with Quercetin

Synthesis of CA encapsulated Pea Protein Nanoparticles by Berry Extract crosslinking (CA-PP-BE-NP): an aqueous solution of Blackberry Extract (BE) for the nanoparticle synthesis was prepared. 25 g of dried purple berry powder was weighed and transferred to a 1 L conical flask. 500 ml distilled water was added to the flask, and the contents were allowed boil for 15 minutes at 100° C. Then the heat was turned off, and the solution was allowed to cool down to room temperature, allowing any remaining insoluble berry fruit powder residues to settle down. The insoluble residues from the purple solution were separated by decantation and then filtration (use a strainer of 75-micron mesh or carry out centrifugation). For the nanoparticle synthesis, the filtered clear purple BE aqueous solution was used. 25 g water soluble Pea Protein (PP) and 13 g caffeine (CA) were weighed and transferred to a 1 L conical flask with a stir bar. 250 mL of the BE extract aqueous solution and 250 mL distilled water were added to the flask, and the mixture stirred overnight. The resulting CA encapsulated Pea Protein Nanoparticles by Berry Extract crosslinking (CA-PP-BE-NP) were characterized by TEM, DLS size and ZP measurements. The CA-PP-BE-NP solution was refrigerated (5±3° C.) for extending shelf life.

Crosslinking was investigated using naturally occurring plant-based materials from blackberries. Quercetin (QUE), a phytochemical present in berries could be extracted from berry extracts. The hydroxyl groups in QUE are effective at crosslinking proteins. CA encapsulated water-soluble Pea Protein Nanoparticles with Berry extract crosslinking (CA-PP-BE-NP) were prepared. Centrifugation/filtration was used to eliminate any insoluble residues following nanoparticle production. CA-PP-BE-NP average size was 386 nm (determined by DLS) and average zeta potential was-14 mV (Table 8b), indicating reasonable stability. A polydispersity index (PDI) of 0.4 suggested nanoparticles of fairly uniform size (unimodal distribution). TEM was used to investigate the size of nanoparticles using the negative staining technique nanoparticle (FIG. 27C). It showed a particle size in the range ˜200 nm. CA-PP-BE-NP was formulated and fully characterized to define quality control parameters for scale-up and commercial manufacturing. Initially, nanoparticles on a small scale (˜10 mL) were formulated. After establishing characterization and quality control parameters, it was successfully scaled up to ˜500 mL to test large scale production. Large scale batches demonstrated reproducible scaling-up and characterization parameters. These experiments had aided in the definition of quality control parameters for usage at scale-up and commercial level productions.

TABLE 8b
Characterization of CA encapsulated Pea Protein Nanoparticles by Berry

| | | | | | | DLS | | |
|---|---|---|---|---|---|---|---|---|
| Serial | PP | CA | BE | Water | PP:CA | Size | | ZP |
| No. | (mg/mL) | (mg/mL) | (mg/mL) | (mL) | Ratio | (nm) | PDI | (mV) |
| 1 | 50 | 25 | 5^{a} | 5 | 2:1 | 295 | 0.2 | −16 |
| 2 | 50 | 25 | 250^{a} | 250 | 2:1 | 476 | 0.5 | −12 |

^{a}= Berry powder (50 mg/mL) boiled at 100 degrees for berry extract (BE)

### Example 15—Crosslinking of Pea Protein with Quercetin and Encapsulation of Theacrine Molecules in Pea Protein Crosslinked with Quercetin

Synthesis of TH encapsulated Pea Protein Nanoparticles by Berry Extract crosslinking (TH-PP-BE-NP): an aqueous solution of Blackberry Extract (BE) for the nanoparticle synthesis was prepared. 25 g of dried purple berry powder was weighed and transferred to a 1 L conical flask. 500 ml distilled water was added to the flask, and the contents were allowed boil for 15 minutes at 100° C. Then the heat was turned off, and the solution was allowed to cool down to room temperature, allowing any remaining insoluble berry fruit powder residues to settle down. The insoluble residues from the purple solution were separated by decantation and then filtration (use a strainer of 75-micron mesh or carry out centrifugation). For the nanoparticle synthesis, the filtered clear purple BE aqueous solution was used. 25 g water soluble Pea Protein (PP) and 13 g theacrine (TH) were weighed and transferred to a 1 L conical flask with a stir bar. 230 mL of the BE extract aqueous solution and 230 ml distilled water were added to the flask, and the mixture stirred overnight. The resulting TH encapsulated Pea Protein Nanoparticles by Berry Extract crosslinking (TH-PP-BE-NP) were characterized by TEM, DLS size and ZP measurements. The TH-PP-BE-NP solution was refrigerated (5±3° C.) for extending shelf life.

Crosslinking was investigated using naturally occurring plant-based materials from blackberries. Quercetin (QUE), a phytochemical present in berries could be extracted from berry extracts. The hydroxyl groups in QUE are effective at crosslinking proteins. TH encapsulated water-soluble Pea Protein Nanoparticles with Berry extract crosslinking (TH-PP-BE-NP) were prepared. Centrifugation/filtration was used to eliminate any insoluble residues following nanoparticle production. TH-PP-BE-NP average size was 360 nm (determined by DLS) and average zeta potential was −15 mV (Table 8c), indicating reasonable stability. A polydispersity index (PDI) of 0.3 suggested nanoparticles of uniform size (unimodal distribution). TEM was used to investigate the size of nanoparticles using the negative staining technique nanoparticle (FIG. 27D). It showed a particle size in the range ˜100 nm. TH-PP-BE-NP was formulated and fully characterized to define quality control parameters for scale-up and commercial manufacturing. Initially, nanoparticles on a small scale (˜10 mL) were formulated. After establishing characterization and quality control parameters, it was successfully scaled up to ˜500 mL to test large scale production. Large scale batches demonstrated reproducible scaling-up and characterization parameters. These experiments had aided in the definition of quality control parameters for usage at scale-up and commercial level productions.

TABLE 8c
Characterization of TH encapsulated Pea Protein Nanoparticles by Berry

| | | | | | | DLS | | |
|---|---|---|---|---|---|---|---|---|
| Serial | PP | TH | BE | Water | PP:TH | Size | | ZP |
| No. | (mg/mL) | (mg/mL) | (mg/mL) | (mL) | Ratio | (nm) | PDI | (mV) |
| 1 | 50 | 25 | 5^{a} | 5 | 2:1 | 352 | 0.3 | −15 |
| 2 | 50 | 25 | 230^{a} | 230 | 2:1 | 368 | 0.3 | −15 |

^{a}= Berry powder (50 mg/mL) boiled at 100 degrees for berry extract (BE)

### Example 16—Crosslinking of Pea Protein with Resveratrol and Encapsulation of Ketone Molecules in Pea Protein Crosslinked with Resveratrol

Synthesis of KM encapsulated Pea Protein Nanoparticles by Grape Extract crosslinking (KM-PP-GE-NP): 50 g whole grapes and 1000 mL distilled water was blended together, and the contents were brought to a boil for 15 minutes at 100° C. Then the heat was turned off, and the solution was cooled to room temperature, allowing any remaining insoluble grape residue to settle down. The insoluble residue from the clear solution was separated by decantation and then filtration (use a strainer of 75-micron mesh or carry out centrifugation). The filtered clear grape extract aqueous solution was utilized for the synthesis of nanoparticle. 50.0 g water soluble Pea Protein (PP) and 300.0 g Ketone Molecule (KM) were weighed and transferred to a 2 L conical flask with a stir bar. 1000 mL of the GE extract aqueous solution was added to the flask, and the contents were boiled for 15 minutes at 100° C. Then, while stirring, 333 mL of absolute ethanol was added to the flask. The reaction mixture contents were stirred overnight. The resulting KM encapsulated Pea Protein Nanoparticles by Grape Extract crosslinking (KM-PP-GE-NP) was characterized by TEM, DLS size and ZP measurements. The KM-PP-GE-NP solution was refrigerated (5° C.±3° C.) for extending shelf life. Blank Pea Protein Nanoparticles by Grape Extract crosslinking (PP-BE-NP) was synthesized following the same procedure without addition of KM.

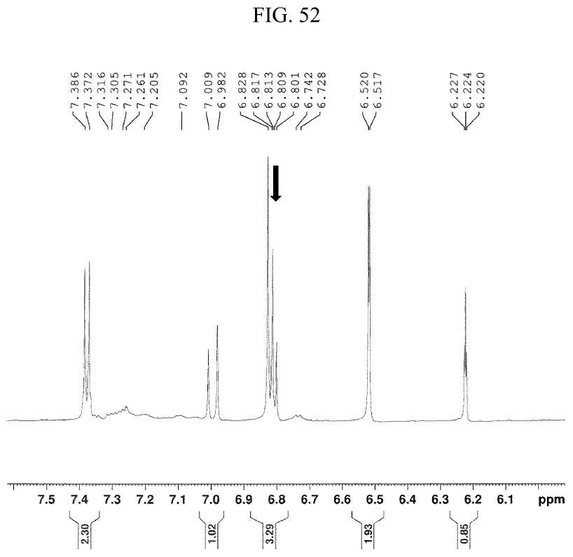

FIG. 52 showed a ¹H NMR (expanded) of resveratrol and water-soluble Pea Protein (PP) reaction mixture in CD₃OD/D₂O (arrows indicate change in proton).

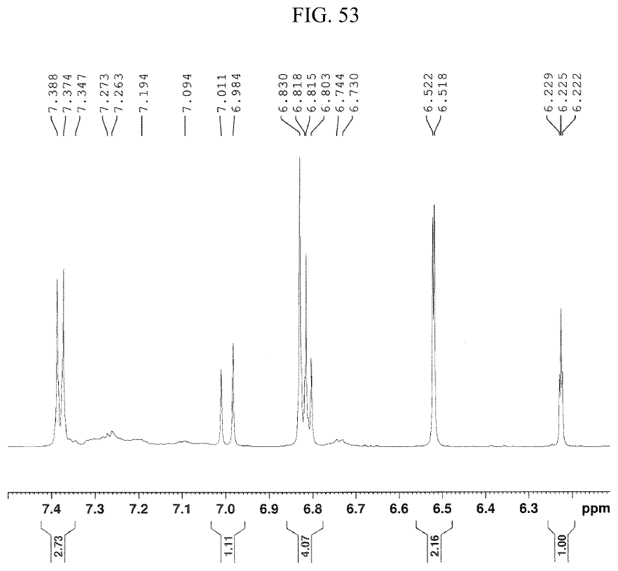

FIG. 53 showed a ¹H NMR (expanded) of resveratrol, water soluble Pea Protein (PP) and KM reaction mixture in CD₃OD/D₂O.

In the resveratrol proton NMR, the 6.8-6.7 ppm region peaks showed a significant change in peak splitting pattern and a slight decrease in peak at 6.2 ppm after reacting with the pea protein (FIGS. 51 and 52). NMR analysis clearly showed distinct change in resveratrol indicating covalent interaction with the protein opening crosslinking pathways. The aromatic ring protons of the phytochemical showed distinct changes indicating covalent interaction with the protein. These changes led to crosslinking with protein amino functional groups. In the resveratrol proton NMR, the 6.8-6.7 ppm region peaks showed a significant change in peak splitting pattern and a slight decrease in peak at 6.2 ppm after reacting with the pea protein indicating covalent interaction with the protein. The carbon NMRs showed corresponding changes.

Resveratrol was a well know antioxidant, with electroactive hydroxy groups facilitated by a planar structure due to the double bond. Also, the grape extract containing the resveratrol was a phytochemical cocktail containing various other active ingredients which can also play a role in the crosslinking and encapsulation process whilst adding nutraceutical value to the formulation.

The framework of Grape Extract (GE) was investigated for nano-encapsulation of KM. Grape extract contains flavonoids and resveratrol, among other polyphenols. The KM encapsulated Pea protein grape extract crosslinked nanoparticles (KM-PP-GE-NP) were produced by combining an aqueous solution of GE, Pea protein, and KM stirring overnight. KM was absorbed or incorporated into the grape framework, which forms a nano-matrix. The size of KM-PP-GE-NP was between 499-607 nm, with an average size of 553 nm (using DLS) and the average zeta potential was determined to be −10 mV (Table 9), showing reasonable stability. A polydispersity index (PDI) of 0.2 indicated that the size of the nanoparticles were uniform and exhibited a unimodal distribution. TEM was used to investigate the size and morphology of nanoparticles using negative staining technique (FIG. 27B). It showed good particle size in the range ˜200-600 nm. KM-PP-GE-NP was formulated and completely characterized to establish quality control criteria for scale-up and commercial productions. Initially, small scale (˜10 mL) nanoparticles were formulated, thereafter production was scaled-u to ˜1 L after establishing characterization and quality control parameters. For the large-scale synthesis, KM from several batches was utilized for the large-scale synthesis. Large scale batches demonstrated reliable scaling up and characterization parameters. These investigations aided in the definition of quality control parameters for usage at scale-up and commercial level productions.

TABLE 9
Characterization of KM encapsulated Pea Protein Nanoparticles by Grape

| | | | Grape | | | | | |
|---|---|---|---|---|---|---|---|---|
| Serial | PP | KM | extract | Water | Ethanol | DLS | | ZP |
| No | (mg/mL) | (mg/mL) | (mL) | (mL) | (mL) | size | PDI | (mV) |
| 14 | 50 | 300 | 10^{a} | 0 | 2 | 499 | 0.2 | −9 |
| 15 | 50 | 300 | 10^{b} | 0 | 2 | 607 | 0.2 | −10 |
| 16 | 50 | 0 | 10^{a} | 10 | 2 | 339 | 0.5 | −4 |

^{a}= Grape extract (25 mg/mL) boiled at 100 degrees
^{b}= Grape extract (50 mg/mL) boiled at 100 degrees

### Example 17—Encapsulation of Ketone Molecules in Soy Protein Isolate

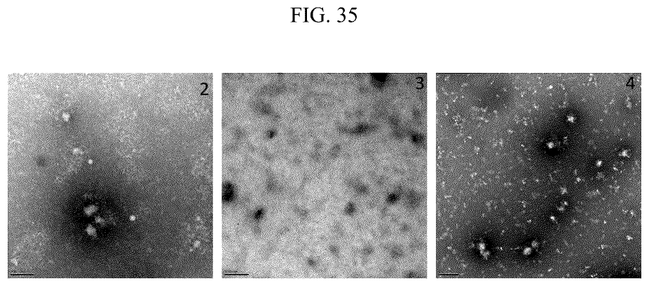

KM-encapsulated Soy Protein Isolate Nanoparticles synthesis and characterization: Soy Protein Isolate (SPI) framework was explored for nano-encapsulation of KM by desolvation method. Since soy protein was explored at the very beginning, glutaraldehyde was used for crosslinking to check for nanoparticle formation before utilizing plant-based phytochemicals. But mango peel, tea, berry, and grape extracts can also be used for crosslinking the nanoparticles. KM loaded Soy Protein Isolate Nanoparticles (KM-SPI-NP) was prepared by ethanol desolvation method. KM-SPI-NPs were formulated and fully characterized to define quality control parameters for use in scale-up and commercial level productions. Various KM concentrations were explored for nanoparticle synthesis. KM-SPI-NP size was between 151-230 nm (using DLS). Polydispersity index (PDI) of ˜0.2 indicated nanoparticles of uniform size (unimodal distribution). They displayed a zeta potential between 35 to 53 mV indicating very good stability (Table 10). Using negative staining technique nanoparticle core size was explored by TEM (FIG. 35).

TABLE 10
Characterization of KM encapsulated Soy Protein Isolate
Nanoparticles by ethanol desolvation method (KM-SPI-NP)

| Serial | SP | Water | KM | Ethanol | SPI:KM | Water:Ethanol | DLS Size | | ZP |
|---|---|---|---|---|---|---|---|---|---|
| No. | (mg/ml) | (ml) | (mg/ml) | (ml) | Ratio | Ratio | (nm) | PDI | (mV) |
| 1 | 15 | 10 | 3 | 3 | 1:0.2 | 3:1 | 199 | 0.2 | −53 |
| 2^{a} | 16 | 10 | 25 | 3 | 1:1.6 | 3:1 | 151^{c} | 0.3 | −35 |
| 3^{b} | 15 | 10 | 124 | 3 | 1:8.3 | 3:1 | 167^{c} | 0.3 | −45 |
| 4 | 16 | 10 | — | 3 | — | 3:1 | 230 | 0.2 | −52 |

^{a}= 100 μL of 5M NaOH was added to change pH 5 to pH 7 and solubilize SPI
^{b}= 200 μL of 5M NaOH was added to change pH 4 to pH 7 and solubilize SPI
^{c}= 20 μL of 25% aqueous glutaraldehyde used for crosslinking

### Example 18—Encapsulation of Ketone Molecules in Okra Extract Crosslinked with Okra Extract

Synthesis of KM encapsulated Okra Extract Nanoparticles (KM-OE-NP): 10 g Okra Extract (OE) powder and 25 g Ketone Molecule (KM) were weighed and transferred to a 1 L conical flask with a stir bar. 500 ml distilled water was added to the flask and the contents were stirred for an hour. 2 g ascorbic acid, a preservative, was weighed and transfer to the flask and stirred overnight. The resulting KM encapsulated Okra Extract (KM-OE-NP) was characterized by TEM, DLS size and ZP measurements. The KM content was estimated through GC-MS analysis and neat KM standard calibration. The KM-OE-NP solution was refrigerated (5° C.±3° C.) for extending shelf life. Blank Okra Extract (OE) without addition of KM was utilized for comparison with KM-OE-NP.

### Example 19—Encapsulation of Ketone Molecules in Okra Extract

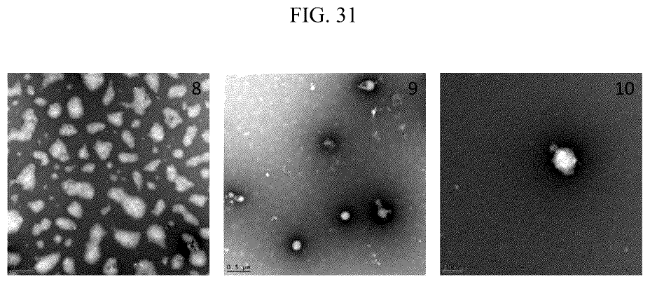

Okra Extract (OE) framework was explored for nano-encapsulation of KM. By mixing aqueous solution of OE and KM and stirring overnight KM okra extract nanoparticles were prepared. KM was absorbed or embedded into the okra mucilage or gel, a form of nano-matrix. KM-OE-NP size was 158 nm (using DLS) and showed a zeta potential of −22 mV (Table 11) indicating reasonable stability. Polydispersity index (PDI) of 0.6 indicates nanoparticles of not very uniform size (away from unimodal distribution). Using negative staining technique nanoparticle core size was explored by TEM (FIG. 31). It showed good particles with size in the ˜100-500 nm range. KM-OE-NP was formulated and fully characterized to define quality control parameters for use in scale-up and commercial level productions. Initially small scale (˜10 mL) nanoparticles were formulated. After establishing characterization and quality control parameters it was scaled up to ˜1 L. KM from various batches were utilized for the large-scale synthesis. Ascorbic acid was added as a preservative as well. Therefore, there was a change in the size and zeta potential. The size was 280 nm (using DLS) and showed a zeta potential of −9 mV (Table 11). Large scale batches showed reliable scale-up and characterization parameters. These experiments had helped to define quality control parameters for use in scale-up and commercial level productions.

Blank Okra Extract: Blank aqueous okra extract with no KM was also characterized for comparison. It showed a size of 82 nm (using DLS) and a zeta potential of −27 mV (Table 11) indicating good stability. PDI of 1.0 indicated an okra gel nano-matrix of non-uniform size (multimodal distribution). Using negative staining technique okra extract core size was explored by TEM (FIG. 31). It showed sparse particles with size in the ˜100 nm range. KM encapsulated Okra Extract Nanoparticles (KM-OE-NP) showed more particles with better polydispersity value in comparison to blank okra extract.

TABLE 11
Characterization of KM Okra Gel Nanoparticles (KM-OE-NP)

| | | | Ascorbic | | | DLS | | |
|---|---|---|---|---|---|---|---|---|
| Serial | OE | KM | Acid | Water | OE:KM | Size | | ZP |
| No. | (mg/mL) | (mg/mL) | (mg/mL) | (mL) | Ratio | (nm) | PDI | (mV) |
| 8 | 22 | 56 | 0 | 10 | 1:3 | 158 | 0.6 | −22 |
| 9 | 20 | 50 | 4^{a} | 500 | 1:3 | 280 | 0.6 | −9 |
| 10 | 20 | 0 | 0 | 10 | 1:0 | 82 | 1.0 | −27 |

^{a}= ascorbic acid added as a preservative for the 500 ml scale

GC-MS analysis of KM encapsulated Okra Extract Nanoparticles (KM-OE-NP): The amount of KM in the nanoparticle formulation was established using GC-MS analysis. It was found to be quantitative indicating 100% KM encapsulation. A high KM encapsulation efficiency may be important for bio-delivery of KM. The GC-MS column provided good resolution of the starting materials and various glycerides. Quantification of KM can be carried out by using the glyceryl tributyrate (GTB) standard and obtaining a calibration curve. The curve should have good linearity and the analysis concentration should fall within the calibration range. GC-MS can be used for quality control of the KM encapsulation process.

### Example 20—Encapsulation of NR Molecules in Okra Extract Crosslinked with Okra Extract

Synthesis of NR-encapsulated okra extract nanoparticles (NR-OE-NP): 10 g Okra Extract (OE) powder (Table 3) and 25 g NR were weighed and transferred to a 1 L conical flask with a stir bar. 500 ml distilled water was added to the flask and the contents were stirred for an hour. 2 g ascorbic acid, a preservative, was weighed and transferred to the flask and stirred overnight. The resulting NR encapsulated Okra Extract (NR-OE-NP) was characterized by TEM, DLS size, and ZP measurements. The NR-OE-NP solution was refrigerated (5° C.±3° C.) for extending shelf life. Blank Okra Extract (OE) without addition of NR was utilized for comparison with NR-OE-NP.

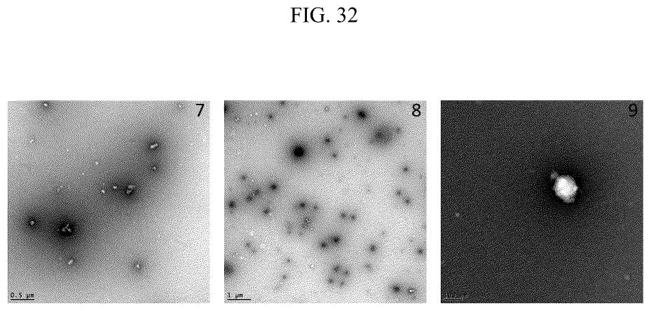

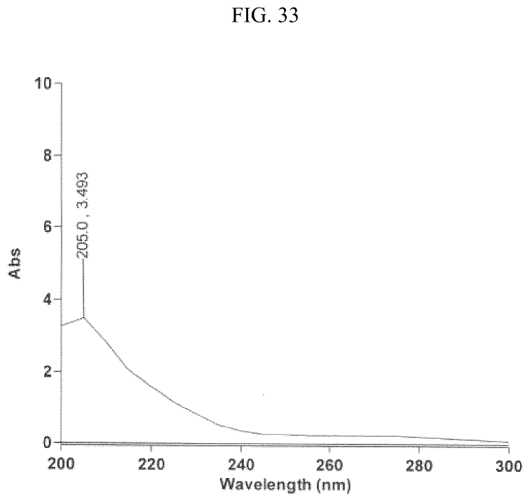

Okra Extract (OE) framework was explored for nano-encapsulation of NR. By mixing aqueous solution of OE and NR and stirring overnight NR okra extract nanoparticles were prepared. NR was absorbed or embedded into the okra mucilage or gel, a form of nano-matrix. NR-OE-NP size was 149 nm (using DLS) and showed a zeta potential of −17 mV (Table 12) indicating reasonable stability. Polydispersity index (PDI) of 0.5 indicated nanoparticles of not very uniform size (away from unimodal distribution). Using negative staining technique nanoparticle core size was explored by TEM (FIG. 32). It showed good particles with size in the ˜200 nm range. NR-OE-NP was formulated and fully characterized to define quality control parameters for use in scale-up and commercial level productions. Initially small scale (˜10 mL) nanoparticles were formulated. After establishing characterization and quality control parameters it was scaled up to ˜1 L. NR from various batches were utilized for the large-scale synthesis. Ascorbic acid was added as a preservative as well. However, there was not much change in the size and zeta potential. The size was 162 nm (using DLS) and showed a zeta potential of −9 mV (Table 8). Large scale batches showed reliable scale-up and characterization parameters. These experiments had helped to define quality control parameters for use in scale-up and commercial level productions.

Blank Okra Extract: Blank aqueous okra extract with no NR was also characterized for comparison. It showed a size of 82 nm (using DLS) and a zeta potential of −27 mV (Table 12) indicating good stability. PDI of 1.0 indicated an okra gel nano-matrix of non-uniform size (multimodal distribution). Using negative staining technique okra extract core size was explored by TEM (FIG. 32). It showed sparse particles with size in the ˜100 nm range. NR encapsulated Okra Extract Nanoparticles (NR-OE-NP) showed more particles with better polydispersity value in comparison to blank okra extract.

TABLE 12
Characterization of NR Okra Gel Nanoparticles (NR-OE-NP)

| | | | Ascorbic | | | DLS | | |
|---|---|---|---|---|---|---|---|---|
| Serial | OE | NR | Acid | Water | OE:NR | Size | | ZP |
| No. | (mg/mL) | (mg/mL) | (mg/mL) | (mL) | Ratio | (nm) | PDI | (mV) |
| 7 | 22 | 52 | 0 | 10 | 1:3 | 149 | 0.5 | −17 |
| 8 | 20 | 50 | 4^{a} | 500 | 1:3 | 162 | 0.6 | −9 |
| 9 | 20 | 0 | 0 | 42 | 1:0 | 82 | 1.0 | −27 |

^{a}= ascorbic acid added as a preservative for the 500 ml scale

### Example 21—Folin-Ciocalteu Analysis for Total Phenols of KM Encapsulated Pea Protein Nanoparticles by Plant-Based (Mango Peel, Tea, Berry, and Grape Extracts) Crosslinking

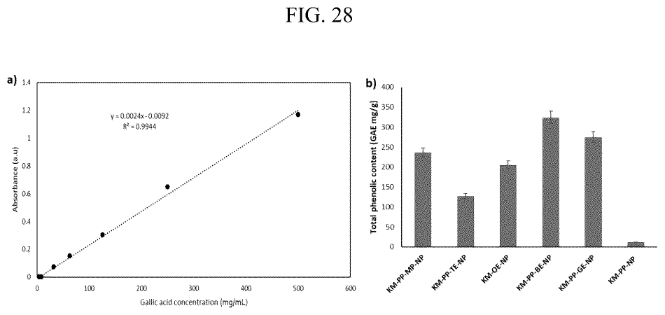

Total phenolic content can be an important component of beverages, supplements, functional foods, food additives, and nutraceutical industries, providing flavor, color, and sensory qualities such as bitterness and astringency, scent, and haze development during storage. Phenols can exhibit antioxidant activity due to their extensive, conjugated x-electron systems which facilitate the donation of electrons or hydrogen atoms to free radicals from the hydroxyl moieties. The oxygen radical absorbance capacity (ORAC) of whole foods, juices, and food additives was previously utilized as the industry standard for determining the antioxidant strength of these foods. The ORAC method made use of the peroxyl radical, the most abundant free radical found in the human body. Long-term consumption of plant-based foods enriched in phytochemicals/polyphenolic substances exhibit physiological functionality in the human diet is also associated with a reduced risk of developing chronic diseases induced by oxidative stress. As a result, they limit the growth of carcinogenic tumors, decrease the risk for cardiovascular disease, and have anti-bacterial, anti-inflammatory, anti-obesity, anti-diabetes, anti-aging, anti-spasmodic, and anti-diarrheic properties. In addition, the energy-enhancing KM within the nanoencapsulation platform contains a number of health-promoting constituents, including antioxidant polyphenols from mango peel, tea, berry, and grape extracts. Antioxidant-rich products are important because they protect body's cells from the destructive effects of free radicals which can damage proteins, lipids, and DNA, thereby reducing and preventing the development of various degenerative diseases such as cancer and coronary heart disease. Phenols provide health benefits through several mechanisms, including the elimination of free radicals and the protection and regeneration of other dietary antioxidants (e.g., vitamin E) acting as protective, and valuable group of phytoconstituents due to their high antioxidant activity and healing properties. The total phenol content was determined using the Folin-Ciocalteu method, which involved reducing the Folin-Ciocalteu reagent with the polyphenols present in nanoformulation. The total phenol content was quantified using total gallic acid equivalents (GAE), the most frequently used reference molecule. Total phenol content was 236 mg GAE/g for KM-PP-MP-NP, 127 mg GAE/g for KM-PP-TE-NP, 205 mg GAE/g for KM-OE-NP, 324 mg GAE/g for KM-PP-BE-NP, 275 mg GAE/g for KM-PP-GE-NP, and 12 mg GAE/g for KM-PP-NPs as shown in FIG. 28.

### Example 22: Cellular Assay Toxicity Studies for KM and KM Encapsulated Pea Protein Nanoparticles by Tea Extract Crosslinking (KM-PP-TE-NP)

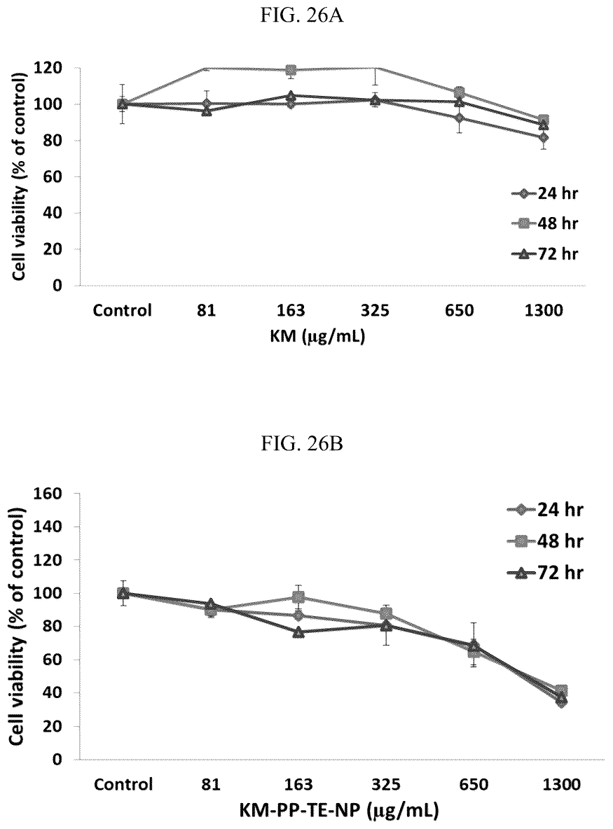

Cellular assay toxicity studies for KM and KM-encapsulated pea protein nanoparticles by Tea Extract crosslinking (KM-PP-TE-NP) were carried out with human aortic endothelial cells (HAEC). The cell proliferation assay kit used 3-(4,5-dimethylthiazol-2-yl)-2,5-diphenyltetrazolium bromide (MTT), a tetrazolium dye for measurement of the cell viability. Using the MTT assay, cell viability was measured for both KM and KM-encapsulated nanoparticles. HAEC after 24, 48 and 72 hours (hr) post incubation with KM showed no toxicity over the concentration range 81-1300 μg/mL. This was shown in FIG. 26A. KM-encapsulated pea protein isolate nanoparticles cross-linked with tea extract (KM-PP-TE-NP) was also synthesized. HAEC after 24, 48 and 72 hr post incubation with KM-PPI-NP showed no toxicity over the concentration range 81-650 μg/mL. This was shown in FIG. 26B. Compared to the free KM, the toxicity concentration for KM encapsulated nanoparticles was slightly lower. This observation inferred enhanced bioavailability of KM in KM-PP-TE-NP. KM concentration in KM-PP-MP-NP was used for plotting the concentration values.

### Example 23—Cellular Assay Toxicity Studies for KM and KM-Encapsulated Pea Protein Nanoparticles by Mango Peel Crosslinking (KM-PP-MP-NP)

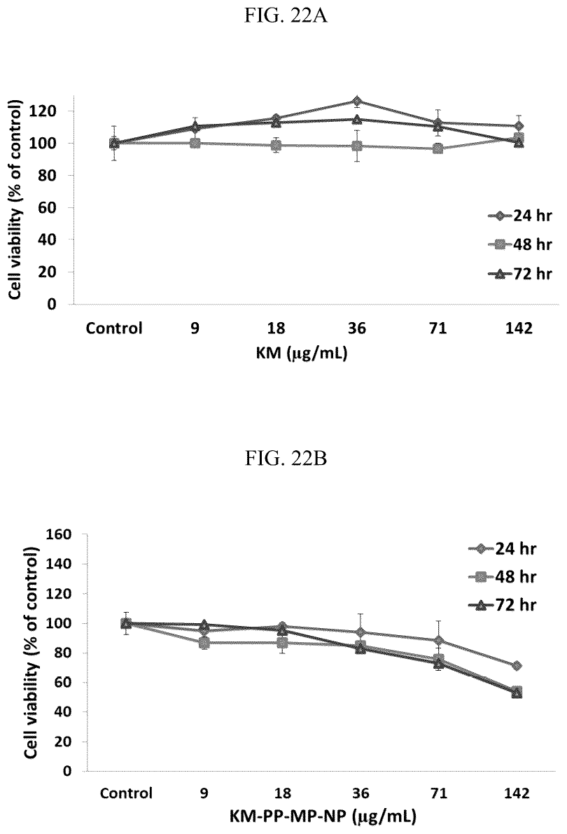

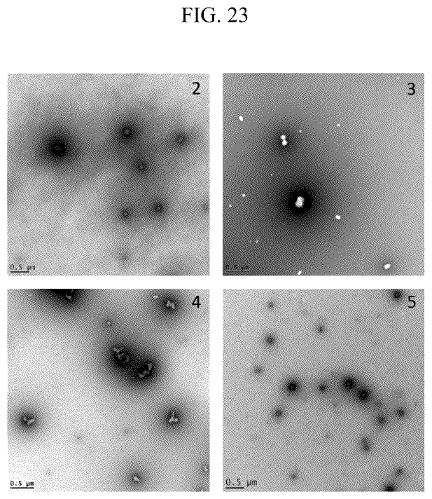

Cellular assay toxicity studies for KM and KM-encapsulated pea protein nanoparticles by Mango Peel crosslinking (KM-PP-MP-NP) were carried out with human aortic endothelial cells (HAEC). The cell proliferation assay kit used 3-(4,5-dimethylthiazol-2-yl)-2,5-diphenyltetrazolium bromide (MTT), a tetrazolium dye for measurement of the cell viability. Using the MTT assay, the cell viability was measured for KM and KM-encapsulated nanoparticles. HAEC after 24, 48 and 72 hours (hr) post incubation with KM showed no toxicity over the concentration range 9-142 μg/mL. This was shown in FIG. 22A. KM encapsulated pea protein isolate nanoparticles cross-linked with mango peel (KM-PP-MP-NP) was also synthesized. HAEC after 24, 48 and 72 hr post incubation with KM-PPI-NP showed no toxicity over the concentration range 9-71 μg/mL. This was shown in FIG. 22B. Compared to the free KM, the toxicity concentration for KM-encapsulated nanoparticles was slightly lower. This observation inferred enhanced bioavailability of KM in KM-PP-MP-NP. KM concentration in KM-PP-MP-NP was used for plotting the concentration values.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of encapsulating a plurality of small molecules, the method comprising:
(a) obtaining plant-derived protein and one or more phytochemicals; and
(b) mixing the plant-derived protein and the one or more phytochemicals with the plurality of small molecules in water to produce a mixture comprising a plurality of particles comprising encapsulated small molecules,
wherein the plurality of small molecules comprises a plurality of ketone molecules;
optionally, wherein the plurality of small molecules further comprises:
(i) a plurality of nicotinamide riboside molecules or nicotinamide riboside analogs;
(ii) a plurality of caffeine molecules; or
(iii) a plurality of theacrine molecules.

2.-7. (canceled)

8. The method of claim 1, wherein the plant-derived protein is pea protein, banana protein, okra protein, or bean protein.

9.-12. (canceled)

13. The method of claim 1, wherein the one or more phytochemicals comprises a mangiferin, a catechin, or a quercetin.

14. The method of claim 13, wherein the one or more phytochemicals comprises quercetin and the weight ratio of the one or more phytochemicals to the plant-derived protein is between 1:10000 and 1:40000; or wherein the one or more phytochemicals comprises mangiferin and the weight ratio of the one or more phytochemicals to the plant-derived protein is between 1:30 and 1:50; or wherein the one or more phytochemicals comprises epigallocatechin gallate and the weight ratio of the one or more phytochemicals to the plant-derived protein is between 1:200 and 1:400.

15.-18. (canceled)

19. The method of claim 1, wherein the one or more phytochemicals comprises a polyphenol, and wherein the polyphenol comprises one or more of a flavonoid and resveratrol.

20. The method of claim 19, wherein the weight ratio of the one or more phytochemicals to the plant-derived protein is between 1:300 and 1:500.

21.-23. (canceled)

24. The method of claim 1, further comprising (c) mixing the mixture with ethanol, wherein (c) is performed by adding the ethanol to the mixture;
(i) at a 1:10 to 1:1 ratio by volume of the ethanol to the mixture;
(ii) at a 1:10 to 1:5 ratio by volume of the ethanol to the mixture; or
(iii) at a 1:4 to 1:2 ratio by volume of the ethanol to the mixture.

25.-30. (canceled)

31. The method of 1, further comprising cross-linking the phytochemicals to the plant protein, wherein:
(i) the phytochemical is crosslinked to the water-soluble plant protein via an imine linkage;
(ii) the cross-linking is performed without an addition of an aldehyde; or
(iii) the cross-linking occurs at one or more functional amino acid groups of the plant protein.

32.-39. (canceled)

40. The method of claim 1, wherein the plurality of small molecules comprise 1,3 Dihydroxypropan-2-yl (R)-3-hydroxybutanoate.

41. The method of claim 1, wherein at least 50% of the small molecules are 1,3 Dihydroxypropan-2-yl (R)-3-hydroxybutanoate.

42. The method of claim 1, wherein at least 50% of the small molecules comprise a glyceryl backbone conjugated to a 3-hydroxybutanoate.

43.-58. (canceled)

59. A composition comprising a small molecule encapsulated within a plant protein cross-linked with one or more phytochemicals; wherein the plurality of small molecules comprises a plurality of ketone molecules.

60. The composition of claim 59, wherein the plant protein is pea protein, banana protein, okra protein, or bean protein.

61.-64. (canceled)

65. The composition of claim 59, wherein the one or more phytochemicals comprises a mangiferin, a catechin, or a quercetin.

66. (canceled)

67. (canceled)

68. The composition of claim 59, wherein the one or more phytochemicals comprises a polyphenol, and wherein the polyphenol comprises one or more of a flavonoid and resveratrol.

69. The composition of claim 59, wherein the one or more phytochemicals are crosslinked to the plant protein via an imine linkage.

70.-72. (canceled)

73. The composition of claim 59, wherein the ketone molecule comprises 1,3 Dihydroxypropan-2-yl (R)-3-hydroxybutanoate.

74. The composition of claim 59, wherein the small molecule further comprises (i) a nicotinamide riboside molecule or an analog thereof, (ii) a caffeine molecule, or (iii) a theacrine molecule.

75.-79. (canceled)

80. The composition of claim 59, wherein the composition has a phenol content greater than 10 mg GAE/g.

81. The composition of claim 80, wherein the composition has a phenol content less than or equal to 350 mg GAE/g.

82.-85. (canceled)
